(19) United States
(12) Patent Application Publication
(10) Pub. No.: US 2019/0291113 A1
(43) Pub. Date: Sep. 26, 2019

(54) METHODS AND SYSTEMS FOR CONDUCTING A CHEMICAL OR BIOLOGICAL REACTION

(71) Applicant: Coyote Bioscience Co., Ltd., Beijing (CN)

(72) Inventors: Jesus Ching, Saratoga, CA (US); Phillip You Fai Lee, South San Francisco, CA (US); Chen Li, Los Gatos, CA (US); Lingguo Du, Lingbao City (CN)

(21) Appl. No.: 16/368,028

(22) Filed: Mar. 28, 2019

Related U.S. Application Data
(63) Continuation of application No. PCT/CN2016/105305, filed on Nov. 10, 2016.

(51) Int. Cl. B01L 3/00; C12Q 1/6844

(52) U.S. Cl. CPC B01L 3/502784; B01L 2300/123; C12Q 1/6844

(57) ABSTRACT

The present disclosure provides methods and systems for analyzing nucleic acids and for conducting chemical and/or biological reactions. Methods and system for droplet generation, guidance, and isolation are also provided.

### CROSS-REFERENCE

This application is a continuation of Patent Cooperation Treaty Application No. PCT/CN2016/105305, filed on Nov. 10, 2016, which is entirely incorporated herein by reference.

### BACKGROUND

Nucleic acid amplification methods may permit selected amplification and identification of nucleic acids of interest from a complex mixture, such as a biological sample. To detect a nucleic acid in a biological sample, the biological sample is typically processed to isolate nucleic acids from other components of the biological sample and other agents that may interfere with the nucleic acid and/or amplification. Following isolation of the nucleic acid of interest from the biological sample, the nucleic acid of interest may be amplified, via, for example, nucleic acid amplification, such as a thermal cycling based approach (e.g., polymerase chain reaction (PCR)). Following amplification of the nucleic acid of interest, the products of amplification may be detected and the results of detection interpreted by an end-user. However, it has been tedious, time consuming and inefficient when multiple or numerous amplification reactions need to be performed.

Droplets have been proposed as partitions to perform chemical and biochemical reactions (e.g., nucleic acid amplification) in confined volumes, and various methods have been developed to generate such droplets. However, these techniques often have problems associated with uneven droplet size and composition, relatively low throughput, and/or unable to generate monodisperse droplets.

In addition, in an appropriate reagent reaction system, nucleic acid amplifications can occur very rapidly. In fact, amplification of nucleic acid molecules in a polymerase chain reaction (PCR) can occur in one to two seconds, or even less than one second per cycle. Therefore, in many situations, the speed of PCR amplification is limited by the performance of the instrumentation (e.g. thermal cycler) rather than the biological reaction itself.

### SUMMARY

Recognized herein is the need for rapid, accurate and high throughput methods, systems and apparatuses to generate droplets of uniform size, shape, and composition, in some cases with an emphasis on droplet composed for the analysis of nucleic acids. Such methods, systems and apparatuses may be useful, for example, in realizing fast sample-to-answer detection and management of diseases detectable via their nucleic acid.

An aspect of the present disclosure provides a method for facilitating a chemical or biological reaction on a biological sample comprising: (a) subjecting a first fluid phase (e.g., a continuous fluid) to flow along a fluid flow path, through at least one opening in a flexible membrane, to a chamber downstream of the membrane; (b) subjecting a second fluid phase (e.g., a fluid comprising the biological sample and/or a fluid immiscible with the first fluid) to flow along the fluid flow path through at least one opening in the membrane to the chamber comprising the first fluid phase; (c) generating a plurality of droplets in the chamber upon the second fluid phase coming in contact with the first fluid phase. A given droplet of the plurality of droplets may comprise the biological sample (or a portion thereof) and reagents necessary for the chemical or biological reaction.

In some embodiments, the first fluid phase and/or the second fluid phase is directed using a flow controller. In some embodiments, the first fluid phase and/or the second fluid phase is directed using positive pressure. In some embodiments, the first fluid phase and/or the second fluid phase is directed using negative pressure. In some embodiments the first fluid phase and/or the second fluid phase is directed using a combination of positive pressure and negative pressure, with said combination of positive and negative pressures being distributed either temporally (e.g., a first pressure at a first time and a second pressure at a second time) or spatially (e.g., a first fluid phase in a first channel directed using a first pressure, such as a positive pressure, and a second fluid phase in a second channel directed using a second pressure, such as a negative pressure). In some embodiments, the first fluid phase or the second fluid phase or both is directed along the fluid flow path under generally laminar flow, though local areas of turbulences are also permissible. In some embodiments, the first fluid phase of the second fluid phase or both is directed along the fluid flow path under Stokes flow.

The first fluid phase of some embodiments comprises reagents necessary for a chemical or biological reaction. The second fluid phase of some embodiments comprises reagents necessary for a chemical or biological reaction. In some embodiments, the first fluid phase comprises an oil. In some embodiments, the first fluid phase comprises a surfactant. In some embodiments the first fluid phase or the second fluid phase or both is a liquid phase.

Some embodiments of the present disclosure have a chemical or biological reaction is nucleic acid amplification. Thus, in some embodiments, the reagents necessary to facilitate a chemical or biological reaction include one or more primers and at least one polymerizing enzyme. Nucleic acid amplification in some embodiments is polymerase chain reaction (PCR). In some embodiments the nucleic acid amplification is isothermal amplification. Some embodiments of the present disclosure comprise two or more types of nucleic acid amplification. The method for facilitating a chemical or biological reaction on a biological sample may further comprise subjecting the given droplet to nucleic acid amplification under conditions necessary to generate amplification product(s) from the biological sample or a portion thereof in the given droplet. In such cases, the nucleic acid amplification is polymerase chain reaction (PCR) or the nucleic acid amplification is isothermal amplification or a combination of the two aforementioned nucleic acid amplification techniques and/or any others known to those of skill in the art. The method for facilitating a chemical or biological reaction on a biological sample may further comprise detecting the amplification product(s) in or from the given droplet.

In some embodiments, the method may further comprise monitoring a temperature of a solution comprising the plurality of droplets. Temperature, in some embodiments, is monitored by detecting a temperature of the solution.

Each of the plurality of droplets of some embodiments has a droplet size from about 0.1 micrometers to about 200 micrometers. Each of the plurality of droplets of some embodiments has a droplet size from about 1 micrometer to 150 micrometers. Each of the plurality of droplets of some embodiments has a droplet size from about 10 micrometers to 100 micrometers. In some embodiments the plurality of droplets is part of an emulsion.

In some embodiments, the chamber is subjected to vibration.

In some embodiments, the membrane is flexible. In some embodiments the membrane may have a portion that is hydrophobic. Hydrophobic membrane embodiments may be hydrophobic as a result of microsurface structures disposed on the membrane or the membrane may be hydrophobic because the membrane comprises a hydrophobic material. In some embodiments the membrane include a lipid bilayer.

In some embodiments, the at least one opening in the membrane permits fluid flow only along a directing leading to the chamber. In some embodiments, the at least one opening includes a one-way valve. The one-way valve of some embodiments is actively controlled. The one-way valve of some embodiments is passively controlled. In some embodiments the at least one opening includes a port protein. The pore protein of some embodiments comprises alpha hemolysin or a variant thereof.

Another aspect of the present disclosure provides a system for conducting a chemical or biological reaction on a biological sample comprising: (a) a fluid flow path in fluid communication with a chamber downstream of a flexible membrane comprising at least one opening; and (b) a controller comprising one or more computer processors that are individually or collectively programmed to (i) subject a first fluid phase (e.g., a continuous fluid) to flow along the fluid flow path, through the at least one opening in the membrane, to the chamber downstream of the membrane; (ii) subject a second fluid phase (e.g., a fluid comprising the biological sample or a portion thereof or a fluid that is immiscible with the first fluid or both) to flow along the fluid flow path through the at least one opening in the membrane to the chamber comprising the first fluid phase; and (iii) generate a plurality of droplets in the chamber upon the second fluid phase coming in contact with the first fluid phase, such that a given droplet of the plurality of droplets comprises the biological sample or reagents necessary for the chemical or biological reaction, or both.

Another aspect provided by the present disclosure provides a method for facilitating a chemical or biological reaction on a biological sample comprising: (a) providing a sample processing unit comprising a fluid flow path in fluid communication with a support comprising a plurality of wells, wherein an individual well of the plurality of wells directs a given droplet of a plurality of droplets to the individual well (e.g., via a hygroscopic material or hygroscopic structure); (b) subjecting the plurality of droplets to flow along the fluid flow path to the plurality of wells, wherein the given droplet of the plurality of droplets comprises the biological sample and reagents necessary for the chemical or biological reaction; and (c) directing the given droplet of the plurality of droplets into the individual well of the plurality of wells.

The hygroscopic material of some embodiments is a polysaccharide.

The method of some embodiments further comprises generating the plurality of droplets upon a first fluid phase coming in contact with a second fluid phase.

In some embodiments, the chemical or biological reaction is nucleic acid amplification. As such, the reagents necessary for the chemical or biological reaction may comprise one or more primers and/or one or more polymerizing enzyme. In some embodiments, the nucleic acid amplification is polymerase chain reaction (PCR). In some embodiments, the nucleic acid amplification is isothermal amplification. In some embodiments, the method may further comprise subjecting the plurality of droplets to nucleic acid amplification under conditions necessary to generate amplification product(s) from the portion of the biological sample in each of the plurality of droplets. In some embodiments, the method may further comprise detecting the amplification product(s) in at least a subset of the plurality of droplets.

In some embodiments, the method may further comprise monitoring a temperature of a solution comprising the given droplet. Moreover, the temperature of some embodiments is monitored by detecting a temperature of the solution.

In some embodiments, each of the plurality of droplets has a droplet size from about 0.1 micrometers to about 200 micrometers. In some embodiments, each of the plurality of droplets has a droplet size from about 1 micrometer to about 150 micrometers. In some embodiments, each of the plurality of droplets has a droplet size from about 10 micrometers to about 100 micrometers.

In some embodiments, the method further comprises sealing the given droplet in the individual well. In some embodiments, the method further comprises providing a fluid phase adjacent to the individual well to seal the given droplet in the individual well. In some embodiments, the fluid phase is an oil phase (e.g., a fluorinated oil).

Another aspect of the present disclosure provides a system for conducting a chemical or biological reaction on a biological sample comprising a sample processing unit (itself comprising a fluid flow path in fluid communication with a support comprising a plurality of wells, wherein an individual well of the plurality of wells directs a given droplet of a plurality of droplets to the individual well via a hygroscopic material) and a controller comprising one or more computer processors that are individually or collectively programmed to (i) subject the plurality of droplets (the plurality of droplets comprising the biological sample and reagents necessary for said chemical or biological reaction) to flow along the fluid flow path and (ii) direct the given droplet of the plurality of droplets into the individual well of the plurality of wells.

Another aspect of the present disclosure provides an apparatus for facilitating a chemical or biological reaction on a biological sample comprising a support that comprises a plurality of wells, wherein an individual well of the plurality of wells comprises a hygroscopic material that (i) directs a given droplet of a plurality of droplets to the individual well, and (ii) retains the given droplet in the individual well during the chemical or biological reaction.

Another aspect of the present disclosure provides a method for facilitating a chemical or biological reaction on a biological sample comprising: (a) providing a sample processing unit (itself comprising a first fluid flow path and a second fluid flow path in fluid communication with a support, wherein the support comprises a plurality of wells, and wherein an individual well of the plurality of wells comprises a first opening adjacent to the first fluid flow path and a second opening adjacent to the second fluid flow path); (b) subjecting the plurality of droplets (a given droplet of the plurality of droplets comprises the biological sample and reagents necessary for said chemical or biological reaction) to flow along the first fluid flow path or the second fluid flow path to the plurality of wells; (c) directing the given droplet of the plurality of droplets from the first fluid flow path or the second fluid flow path into the individual well of the plurality of wells through the first or second opening; and (d) providing a first fluid phase in the first fluid path and a second fluid phase in the second fluid path, thereby retaining the given droplet in the individual well.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example apparatus for generating droplets;

FIGS. 2A-2C illustrates a support system;

FIGS. 3A and 3B illustrate example populations of droplets;

FIG. 4 illustrates a graph demonstrating a signal transmitted by a detectable moiety as a function of temperature;

FIG. 5 illustrates a temperature monitoring system comprising a plurality of temperature indicators;

FIG. 6 illustrates a cross-sectional view of a support system comprising a temperature monitor;

FIG. 7A illustrates a perspective view of an exemplary droplet generating apparatus;

FIG. 7B illustrates a cut perspective view of the exemplary droplet generating apparatus of FIG. 7A;

FIG. 7C illustrates a close-up view of a chamber of the exemplary droplet generating apparatus of FIG. 7A;

FIG. 7D illustrates a cut side view of the exemplary droplet generating apparatus of FIG. 7A;

FIG. 8A illustrates a perspective view of an exemplary embodiment of a support system comprising a plurality of wells;

FIG. 8B illustrates a top view of the flow paths of the exemplary embodiment of the support system comprising a plurality of wells shown in FIG. 8A;

FIG. 8C illustrates a close-up view of a subset of the plurality of wells from the exemplary embodiment of the support system comprising a plurality of wells shown in FIG. 8A;

FIG. 9A illustrates a perspective view of an exemplary droplet generation system comprising a droplet generation apparatus;

FIG. 9B illustrates a cut side view of the exemplary droplet generation system shown in FIG. 9A;

FIG. 9C illustrates a perspective view of the droplet generation apparatus of the droplet generation system shown in FIG. 9A;

FIG. 10 shows an example computer control system that is programmed or otherwise configured to implement methods provided herein;

FIG. 11A shows a plurality of droplets generated by an experimental droplet generation system using a flow rate of 75 microliters per hour;

FIG. 11B shows a plurality of droplets generated by an experimental droplet generation system using a flow rate of 150 microliters per hour;

FIG. 11C shows a plurality of droplets generated by an experimental droplet generation system using a flow rate of 300 microliters per hour;

FIG. 11D shows a plurality of droplets generated by an experimental droplet generation system using a flow rate of 600 microliters per hour;

FIG. 11E shows a plurality of droplets generated by an experimental droplet generation system using a flow rate of 1000 microliters per hour;

FIG. 11F illustrates a graph relating droplet size to the flow rate as determined by the pluralities of droplets seen in FIGS. 11A-11E.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a molecule” includes a plurality of molecules, including mixtures thereof.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “primer extension reaction” generally refers to the denaturing of a double-stranded nucleic acid, binding of a primer to one or both strands of the denatured nucleic acid, followed by elongation of the primer(s).

As used herein, the term “reaction mixture” generally refers to a composition comprising reagents necessary to complete nucleic acid amplification (e.g., DNA amplification, RNA amplification), with non-limiting examples of such reagents that include primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some embodiments, reaction mixtures can also comprise one or more reporter agents.

As used herein, a “reporter agent” generally refers to a composition that yields a detectable signal, the presence or absence of which may be used to detect a chemical or biological reaction. In some cases, reporter agents may bind to initial reactants and changes in reporter agent levels may be used to detect amplified product. In some cases, reporter agents may only be detectable (or non-detectable) as a reaction progresses. A reporter agent may be an optically-active dye (e.g., a fluorescent dye). Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green H, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer HI, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methyl coumarin -3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some cases, a reporter agent may be a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active reporter agents (e.g., dyes) described herein and may also include a quencher capable of blocking the optical activity of an associated dye. Non-limiting examples of probes that may be useful used as reporter agents include TaqMan probes, TaqMan Tamara probes, TaqMan MGB probes, or Lion probes.

In some cases, a reporter agent may be an RNA oligonucleotide probe that includes an optically-active dye (e.g., fluorescent dye) and a quencher positioned adjacently on the probe. The close proximity of the dye with the quencher can block the optical activity of the dye. The probe may bind to a target nucleic acid sequence to be amplified. Upon the breakdown of the probe with the exonuclease activity of a DNA polymerase during amplification, the quencher and dye are separated, and the free dye regains its optical activity that can subsequently be detected.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

Amplification of a nucleic acid may be linear, exponential, or any combination thereof. Non-limiting examples of nucleic acid amplification methods include reverse transcription, primer extension, ligase chain reaction (LCR), helicase-dependent amplification (e.g., amplification that is preceded by contacting the nucleic acid with a helicase), asymmetric amplification, rolling circle amplification, multiple displacement amplification (MDA), polymerase chain reaction (PCR) and variants thereof. Non-limiting examples of PCR variants include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, amplification is achieved with nested nucleic acid amplification. Moreover, amplification of a nucleic acid may be conducted isothermally or may be conducted via one or more temperature cycles (e.g., thermal cycling). Thermal cycling of the solution can be useful for a host of sample processing and/or biological/chemical reactions, including preparation of the biological sample for a nucleic acid amplification reaction and conducting the nucleic acid amplification reaction.

As used herein, the term “components necessary for conducting a chemical or biological reaction” generally refer to a material(s) that are required to complete and/or detect a given chemical or biological reaction on a biological sample. The components can be those necessary for conducting any type of chemical or biological reaction whose progress is initiated, sustained and/or enhanced with the inclusion of heat. Non-limiting examples include nucleic acid amplification reactions, denaturation reactions, cell lysis reactions, enzymatic reactions, reaction involving molecular recognition, and other chemical or biological reactions. Such components can include reactants, catalysts (e.g., enzymes), reaction mediums (e.g., buffer, solvent), reporter agents for reaction detection, and co-factors. Where the chemical or biological reaction is a nucleic acid amplification reaction, the components can be components necessary for the nucleic acid amplification reaction. Components necessary for a nucleic acid amplification reaction include one or more template nucleic acid molecules (e.g., a template nucleic acid molecule derived from a biological sample), one or more primers, one or more polymerizing enzymes, one or more deoxynucleotide triphosphates (dNTPs), co-factors (e.g., cations such as Mg²⁺) and a suitable reaction medium (e.g. buffer).

In some cases, the polymerizing enzyme is a polymerase (e.g., a DNA polymerase) that is capable incorporating nucleotides to a primer in a template directed manner. The polymerase may be any suitable polymerase and multiple polymerases may be implemented. Non-limiting examples of polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof.

As used herein, the terms “denaturing” and “denaturation” are used interchangeably and generally refer to the full or partial unwinding of the helical structure of a double-stranded nucleic acid, and in some embodiments the unwinding of the secondary structure of a single stranded nucleic acid. Denaturation may include the inactivation of the cell wall(s) of a pathogen or the shell of a virus, and the inactivation of the protein(s) of inhibitors. Conditions at which denaturation may occur include a “denaturation temperature” that generally refers to a temperature at which denaturation is permitted to occur and a “denaturation duration” that generally refers to an amount of time allotted for denaturation to occur.

As used herein, the term “elongation” generally refers to the incorporation of nucleotides to a nucleic acid in a template directed fashion. Elongation may occur via the aid of an enzyme, such as, for example, a polymerase or reverse transcriptase. Conditions at which elongation may occur include an “elongation temperature” that generally refers to a temperature at which elongation is permitted to occur and an “elongation duration” that generally refers to an amount of time allotted for elongation to occur.

As used herein, the term “subject,” generally refers to an entity or a medium that has testable or detectable genetic information. A subject may be a person or individual. A subject may be a vertebrate, such as, for example, a mammal. Non-limiting examples of mammals include murines, simians, humans, farm animals, sport animals, and pets. Other examples of subjects include, for example, food, plant, soil, and water. A subject may be a patient or an individual being treated or seeking treatment. A subject may be from a pathogen, such as a virus, bacterium, or microorganism. The target sequence may be from or correspond to a sequence of pathogen, such as a virus, bacterium or microorganism. Target sequences from and/or corresponding to a sequence from a virus may be from and/or correspond to an RNA virus or a DNA virus. In some embodiments, the virus from which a target sequence is taken or to which a target sequence corresponds is selected from the group consisting of human immunodeficiency virus I (HIV I), human immunodeficiency virus II (HIV II), an orthomyxovirus, Ebola virus, Dengue virus, influenza viruses, herpesvirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, Coxsackie virus, and Varicella virus. The influenza virus to which some target sequences correspond (and/or are taken from) include but are not limited to the group consisting of H1N1 virus, H3N2 virus, H7N9 virus and H5N1 virus. The adenovirus to which some target sequences correspond (and/or are taken from) may be adenovirus type 55 (ADV55) or adenovirus type 7 (ADV7). The hepatitis C virus to which some target sequences correspond (and/or are taken from) may be, for example, armored RNA-hepatitis C virus (RNA-HCV). The Coxsackie virus to which some target sequences correspond (and/or are taken from) includes Coxsackie virus A16.

A target sequence of some embodiment is from a pathogenic bacterium or a pathogenic protozoan. The pathogenic bacterium of such embodiments may be a gram-positive or gram-negative pathogenic bacterium. In some embodiments, the pathogenic bacterium is selected from the group consisting of Staphylococcus aureus, Listeria monocytogenes, Escherichia coli, Enterobacter sakazakii, Vibrio Parahemolyticus, and Shigella spp. In some embodiments, the pathogenic bacterium is Mycobacterium tuberculosis. In some embodiments, the pathogenic protozoan is Plasmodium. In some embodiments, the pathogenic bacterium is Salmonella.

As used herein, the terms “incubating” and “incubation” are used interchangeably and generally refer to keeping a sample, a mixture or a solution at certain temperature for a certain period of time, with or without shaking or stirring. An “incubation temperature” generally refers to a temperature at which incubation is permitted to occur. An “incubation time period” generally refers to an amount of time allotted for incubation to occur.

As used herein, the term “fluid” generally refers to a liquid or a gas. A fluid cannot maintain a defined shape and will flow during an observable time frame to fill a container in which it is put. Thus, a fluid may have any suitable viscosity that permits flow. If two or more fluids are present, each fluid may be independently selected among essentially any fluids (liquids, gases, and the like) by those of ordinary skill in the art.

As used herein, the term “aqueous fluid” generally refers to a fluid that is made with, of, or from water, or a fluid that contains water. For example, an aqueous fluid may be an aqueous solution with water as the solvent. An aqueous fluid of the present disclosure may comprise reagents necessary for conducting a desired chemical reaction, e.g., polymerase chain reaction (PCR). Non-limiting examples of aqueous fluid include, but are not limited to, water and other aqueous solutions comprising water, such as cell or biological medium, ethanol, salt solutions, etc.

As used herein, the term “continuous fluid” generally refers to a fluid that forms a continuous flow. A continuous fluid may be a fluid immiscible with an aqueous solution. For example, a continuous fluid may be a non-aqueous fluid made from, with, or using a liquid other than water. Non-limiting examples of continuous fluid include, but are not limited to, oils such as hydrocarbons, silicon oils, fluorine-containing oils (e.g., fluorocarbon oils), organic solvents etc.

As used herein, the term “channel” generally refers to a path that confines and/or directs the flow of a fluid. A channel of the present disclosure may be of any suitable length. The channel may be straight, substantially straight, or it may contain one or more curves, bends, etc. For example, the channel may have a serpentine or a spiral configuration. In some embodiments, the channel includes one or more branches, with some or all of which connected with one or more other channel(s).

As used herein, a “cross-sectional dimension” of a channel may be measured perpendicularly with respect to the general direction of fluid flow within the channel.

As used herein, the use of the term “elastic modulus” may be interpreted as encompassing myriad facets of elasticity including tensile elasticity (Young's modulus), shear moduli, moduli of rigidity, bulk moduli, axial moduli, Lame's parameters (such as the first parameter), P-wave moduli, etc. It may be used to describe homogenous materials, heterogeneous materials, isotropic materials, anisotropic materials, and composite materials. More broadly, “elastic modulus” is used to broadly describe the myriad parameters of the elasticity tensor of a material.

As used herein, the term “droplet” generally refers to an isolated portion of a first fluid (e.g., an aqueous fluid) that is surrounded by a second fluid (e.g., a continuous fluid). An emulsion may include a dispersion of droplets of a first fluid (e.g., liquid) in a second fluid. The first fluid may be immiscible with the second fluid. In some embodiments, the first fluid and the second fluid are substantially immiscible. A droplet of the present disclosure may be spherical or assume other shapes, such as, for example, shapes with elliptical cross-sections. The diameter of a droplet, in a non-spherical droplet, is the diameter of a perfect mathematical sphere having the same volume as the non-spherical droplet. A droplet may include a skin. The skin may form upon heating the droplet. The skin may have a higher viscosity than an interior of the droplet. In some embodiments, the skin may prevent the droplet from fusing with other droplets.

As used herein, the term “sample” generally refers to any sample containing or suspected of containing a nucleic acid molecule. For example, a subject sample may be a biological sample containing one or more nucleic acid molecules. The biological sample may be obtained (e.g., extracted or isolated) from a bodily sample of a subject that may be selected from blood (e.g., whole blood), plasma, serum, urine, saliva, mucosal excretions, sputum, stool and tears. The bodily sample may be a fluid or tissue sample (e.g., skin sample) of the subject. In some examples, the sample is obtained from a cell-free bodily fluid of the subject, such as whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

In some embodiments, a sample is obtained directly from a subject without further processing. In some embodiments, a sample is processed prior to a biological or chemical reaction (e.g., nucleic acid amplification). For example, a lysis agent may be added to a sample holder prior to adding a biological sample and reagents necessary for nucleic acid amplification. Examples of the lysis agent include Tris-HCl, EDTA, detergents (e.g., Triton X-100, SDS), lysozyme, glucolase, proteinase E, viral endolysins, exolysins zymolose, Iyticase, proteinase K, endolysins and exolysins from bacteriophages, endolysins from bacteriophage PM2, endolysins from the B. subtilis bacteriophage PBSX, endolysins from Lactobacillus prophages Lj928, Lj965, bacteriophage 15 Phiadh, endolysin from the Streptococcus pneumoniae bacteriophage Cp-I, bifunctional peptidoglycan lysin of Streptococcus agalactiae bacteriophage B30, endolysins and exolysins from prophage bacteria, endolysins from Listeria bacteriophages, holin-endolysin, cell 20 lysis genes, holWMY Staphylococcus wameri M phage varphiWMY, Iy5WMY of the Staphylococcus wameri M phage varphiWMY, Tween 20, PEG, KOH, NaCl, and combinations thereof. In some embodiments, a lysis agent is sodium hydroxide (NaOH). In some embodiments, the biological sample is not treated with a detergent.

In some embodiments, systems or methods further comprises a detector. During detection, the detector detects a signal from the solution that is indicative of a chemical or biological reaction on the biological sample. In some embodiments, the detector may be integral with the vessel holding a solution. In some embodiments the detector may be angularly separated from the vessel. In some embodiments the detector may be operatively coupled with the vessel. In some embodiments the detector is operatively coupled to at least a first thermal zone such that as a detectable sample is brought into at least a first thermal zone, the detectable sample is detected by the detector. In some embodiments the controller positions the solution in sensing communication with the detector. The solution and the detector may be brought into sensing communication via translation for the solution with respect to the detector (and/or vice versa) or via a rotation of the solution with respect to the detector (and/or vice versa), or any combination thereof. The axes of translation and the axes of rotation may be with respect to any characteristic axis of the detector (e.g., with respect to the axis defined by the optical communication path, with respect to the axis perpendicular to the optical communication path, etc.).

The droplets may include detectable moieties that permit detection of any signals generated from the biological and/or chemical reactions (e.g., nucleic acid amplification reactions). For example, the detectable moieties may yield a detectable signal whose presence or absence is indicative of a presence of an amplified product. The intensity of the detectable signal may be proportional to the amount of amplified product. In some embodiments, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a detectable moiety that yield a detectable signal indicative of the presence of the amplified DNA product and/or the target RNA amplified. The intensity of the detectable signal may be proportional to the amount of the amplified DNA product and/or the original target RNA amplified. The use of a detectable moiety also enables real-time amplification methods, including real-time PCR for DNA amplification.

Detectable moieties may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, detectable moieties bind to initial reactants and changes in detectable moiety levels are used to detect amplified product. In some embodiments, detectable moieties are only detectable (or non-detectable) as nucleic acid amplification progresses. In some embodiments, an optically-active dye (e.g., a fluorescent dye) is used as a detectable moiety, such as any described herein. Non-limiting examples of such dyes that may be used as a detectable moiety include but are not limited to SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a detectable moiety is a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, or Lion probes.

In some embodiments and where a detectable moiety is an RNA oligonucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected.

In some embodiments, a detectable moiety is a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and may be detected. The sequence specificity of the molecular beacon for a target sequence on the amplified product can improve specificity and sensitivity of detection.

In some embodiments, a detectable moiety is a radioactive species. Non-limiting examples of radioactive species include ¹⁴C, ¹²³I, ¹²⁴I, ¹²⁵I, ¹³¹I, Tc99m, ³⁵S, and ³H.

In some embodiments, a detectable moiety is an enzyme that is capable of generating a detectable signal. Detectable signal may be produced by activity of the enzyme with its substrate or a particular substrate in the case the enzyme has multiple substrates. Non-limiting examples of enzymes that may be used as detectable moieties include alkaline phosphatase, horseradish peroxidase, I²-galactosidase, alkaline phosphatase, β-galactosidase, acetylcholinesterase, and luciferase.

In some embodiments, a detectable moiety may comprise a thermal liquid crystal (TLC), also known as a thermochromic liquid crystal, whose color response is a function of temperature. A TLC may comprise a material that changes its reflected color as a function of temperature when illuminated by a light of a first color (e.g., white, infrared, red, orange, yellow, green, blue, violet, ultraviolet). A detectable moiety comprising at least one TLC may reflect light (either visible or invisible) of a first wavelength at a first temperature and reflect light (either visible or invisible) of a second wavelength at a second temperature. In some embodiments, the detectable moiety may be disposed within a strip, a panel, a sheet, a plate, or a sticker. In some embodiments, the detectable moiety may be disposed within at least one droplet (in some embodiments selected from a plurality of droplets) disposed within a system such that the temperature of sample may be measured by detecting the color of the at least one droplet. Detection of the detectable moiety disposed within at least one droplet may be used to calibrate the system (e.g., prompting the controller to direct heat generation and/or cooling, prompting the controller to generate the amount droplets or the rate of droplet generation, etc.).

In some embodiments, the sample is purified (e.g., by filtration, centrifugation, column purification and/or magnetic purification, for example, by using magnetic beads (e.g., super paramagnetic beads)) to obtain purified nucleic acids.

As used herein, the term “about” or “nearly” generally refers to a reasonable variation, e.g. within +/−10%, 9%, 8%, 7%, 6%, 5%, 4%, 3%, 2%, or 1% of a designated amount.

As used herein, the term “overshooting” generally refers to a point or region that is above or below a target or designated point or region. In some examples, in heating, an overshooting thermal zone may be at a temperature that is above a target temperature, and in cooling, an overshooting thermal zone may be at a temperature that is below a target temperature. For example, in heating a solution to 100° C., an overshooting thermal zone at a temperature of about 140° C. may be used. In another example, in cooling a solution to 25° C., an overshooting thermal zone at a temperature of about 0° C. may be used. An overshooting thermal zone may provide a greater temperature drop or temperature change, which may in turn provide a greater rate of heat transfer to provide heating or cooling, as necessary or required.

As used herein, the term “thermal communication” generally refers to a state in which two or more materials are capable of exchange energy, such as thermal energy, with one another. Such exchange of energy may be by way of transfer of energy from one material to another material. Such transfer of energy may be radiative, conductive, or convective heat transfer. The energy may be thermal energy. In some examples, two or more materials that are in thermal communication with one another are in thermal contact with one another, such as, for example, direct physical contact or contact through one or more intermediary materials.

### Droplet Generation

In an aspect of the present disclosure, a method for facilitating a chemical or biological reaction on a biological sample may comprise subjecting a first fluid phase to flow along a fluid flow path, through at least one opening a membrane, to a chamber downstream of the membrane; subjecting a second fluid phase to flow along the fluid flow path through at least one opening in the membrane to the chamber; and generating a plurality of droplets in the chamber when the second fluid phase contacts the first fluid phase. The first fluid phase may be immiscible with the second fluid phase and the second fluid phase may comprise the biological sample, a portion of the biological sample and/or reagents necessary for the chemical or biological reaction. Hence, a given droplet of the plurality of droplets may comprise the biological sample (and/or a portion thereof) and/or reagents necessary for the chemical or biological reaction.

The membrane may be flexible. For instance, the membrane may comprise a material with an elastic modulus of no greater than about 100 gigapascals (GPa), 90 GPa, 80 GPa, 70 GPa, 60 GPa, 50 GPa, 40 GPa, 30 GPa, 20 GPa, 10 GPa, 9 GPa, 8 GPa, 7 GPa, 6 GPa, 5 GPa, 4 GPa, 3 GPa, 2 GPa, 1 GPa, 0.9 GPa, 0.8 GPa, 0.7 GPa, 0.6 GPa, 0.5 GPa, 0.4 GPa, 0.3 GPa, 0.2 GPa, 0.1 GPa, 90 megapascals (MPa), 80 MPa, 70 MPa, 60 MPa, 50 MPa, 40 MPa, 30 MPa, 20 MPa, 10 MPa, 9 MPa, 8 MPa, 7 MPa, 6 MPa, 5 MPa, 4 MPa, 3 MPa, 2 MPa, or 1 MPa, or the value of the elastic modulus may take a value in between any two aforementioned values. The membrane may comprise a material with an elastic modulus between about 0.1 GPa to about 5 GPa. Materials that may comprise the membrane include but are not limited to: acetal copolymer, acetal homopolymer, acrylonitrile butadiene styrene (ABS), aluminum, bismaleimide, bismuth, boron, carbide, carbide foam, carbon, carbon foam, carbon nanofibers, cellulose, cesium, cesium iodide, copper, cyanoacrylate, ethylene chlorotrifluoroethylene (ECTFE), ethylene vinyl alcohol, furan, glass, graphite, high-density polyethylene, low-density polyethylene, maleimide, melamine, methacrylate, nylon, phenol formaldehydes, phenolics, plastarch, polyactic acid, polyamides, polyaryletherketone (PAEK), polycarbonate, polychlorotrifluoroethylene, polyepoxide, polyester, polyetheretherketone (PEEK), polyetherimide, polyethylene, polyimide, polymethyl methacrylate (PMMA), polyolefin, polypropylene, polystyrene, polysulfone, polytetrafluoroethylene (PTFE), polyurethane, polyvinyl chloride, polyvinylidene chloride, polyvinylidinefluoride (PVDF), rubidium, silicone, thermoplastic, thermoplastic elastomers, and urea-formaldehyde. Alloys and/or composites of the aforementioned materials may also be used.

The structure and/or geometrical configuration of the membrane may aid in its flexibility. For example, the membrane may have a thickness of about 5 nanometers (nm), 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 micrometer (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeter (mm), 2 mm, 3 mm, 4 mm, 5 mm, 6 mm, 7 mm, 8 mm, 9 mm, 1 centimeter (cm), 2 cm, 3 cm, 4 cm, 5 cm, 6 cm, 7 cm, 8 cm, 9 cm, 10 cm, or the thickness of the membrane may take on any value between any two aforementioned values. There are other approaches by which to make the membrane flexible, such as, for example, using divots along the membrane, channels extending along one surface of the membrane, portions of the membrane comprising at least a first material and a second material, the second material having greater flexibility than the first material, etc.

The at least one opening of the membrane may take on any shape including but not limited to a circle, an oval, an ellipse, a triangle, a square, a pentagon, a hexagon, a polygon, or any profile that may be described as the sum of any number of sine and cosine functions. Opening(s) within the membrane may have a diameter no greater than about 1 mm, 900 micrometers (μm), 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 5 μm, 1 μm, 0.5 μm, or 0.1 μm, or the size of the opening(s) of the membrane may take on a value in between any two of the aforementioned values. The opening(s) within the membrane may have a diameter from approximately 1 μm to about 50 μm. Opening(s) may have a uniform cross-sectional area and/or shape as they extend from one side of the membrane to another. In some embodiments, opening(s) may a cross-sectional area and/or shape that varies along their length from one side of the membrane to another (e.g., the cross-sectional area may increase from one side to another, the cross-sectional area may decrease from one side to another, etc.). At least one opening of the membrane may permit fluid(s) to flow along in one direction only (in the direction of the chamber, for instance). For instance, at least one opening in the membrane may include a one-way valve (such as a check valve). Examples of possible valves include but are not limited to aspin valves, ball valves, ball cock valves, bibcock valves, blast valves, Boston valves, butterfly valves, ceramic disc valves, check valves, choke valves, clapper valves, cock valves, demand valves, diaphragm valves, double beat valves, double check valves, duckbill valves, flipper valves, flow control valves, foot valves, four-way valves, freeze plug valves, freeze seal valves, gas pressure valves, gate valves, globe valves, Heimlich valves, knife valves, Lamer-Johnson valves, leaf valves, needle valves, pilot valves, pinch valves, piston valves, plug valves, plunger valves, poppet valves, poppet valves, pressure regulator, pressure reducing valves, presta valves, reed valves, relief valves, rocker valves, rotary valves, rotolock valves, rupture valves, saddle valves, safety valves, sampling valves, Schrader valves, solenoid valves, spool valves, stopcock valves, swirl valves, Tesla valves, thermal expansion valves, thermal expansion valves, thermostatic mixing valves, thermostatic radiator valves, vacuum breaker valves, or variants thereof.

For those embodiments comprising at least two openings, the openings may be spaced apart from each other in any pattern such as a linear pattern, a grid-like pattern, a radial-like pattern, a spiral-like pattern, a Poisson-distribution-based pattern, etc. The spacing(s) between an opening and its neighboring opening(s) may be uniform or it may vary. Spacing between an opening and its nearest neighbor may be about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1 mm, or the spacing between an opening and its nearest neighbor may take on a value between any two of the aforementioned values. The distribution of openings may be symmetric or asymmetric.

A hydrophobic coating may be applied to the membrane, such that at least a portion of the membrane (e.g., a first surface, a second surface different from the first surface, half of a first surface, at least one opening, the region(s) near opening(s), etc.) may comprise a hydrophobic coating. The membrane itself may be hydrophobic and/or comprise a hydrophobic material. Such hydrophobicity may an inherent property of the material comprising the membrane and/or it may arise as a function of surface features (such as microstructures) of at least a portion of the membrane. Materials that may be used to facilitate hydrophobicity on at least a portion of the membrane include but are not limited to: acrylics, amides, block copolymers, carbonates, dienes, esters, ethers, fluorocarbons, imides, olefins, styrenes vinyls, vinyl acetals, vinyl esters, vinyl eths, vinyl ketones, vinylidene chlorides, vinylpryrolidone polymers, and vinylpyridines.

Furthermore, the membrane may comprise biological materials to confer flexibility, hydrophobicity, or other desired properties (such as biocompatibility, boundary layer development, etc.). For example, the membrane may comprise a lipid bilayer. Optionally or as an alternative, the membrane or at least a portion of the membrane (e.g., an opening) may comprise at least one pore protein, such as alpha hemolysin or a variant thereof.

Composite materials (a material comprising two or more constituent materials of different physical and/or chemical properties) may be used for the membrane, so long as at least one material of the composite material used for the membrane has an elastic modulus value between about 1 MPa and 100 GPa. The membrane may comprise any combination of the materials described herein, variants of the materials described herein, alloys of the materials described herein, and/or the products of reactions involving the materials described herein.

The membrane may intersect the fluid flow path. The fluid flow path and the membrane may intersect temporarily, periodically, permanently, and/or operatively. The intersection of the fluid flow path (as defined by a flow path vector) and the membrane (as defined by a membrane vector) may form an angle of about 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, or 180°, or the angle of intersection between the fluid flow path and the membrane may take on any value between any two aforementioned values. The intersection of the fluid flow path and the membrane may change over time such that at a first time a fluid (e.g., a first fluid, a second fluid, etc.) may flow at a first angle with respect to the membrane and at a second time the fluid may flow at a second angle with respect to the membrane. As a non-limiting example, at the first time the fluid may flow at an angle approximately perpendicular to the membrane at the second time the fluid may flow at an angle approximately parallel to the membrane.

A fluid of any embodiment may be directed by or using a controller. The first fluid and/or second fluid phase, for instance, may be directed using a flow controller. The first fluid and/or second fluid may be caused to flow via positive pressure and/or negative pressure at any given time. A pump may be used to cause one or more fluids to flow. Pumps that may be used include but are not limited to a capillary pump, a centrifugal pump, a diaphragm pump, a duplex pump, a gear pump, a jet pump, a lobe pump, a multiplex pump, a peristaltic pump, a piston plunger pump, propeller pump, a reciprocating pump, a rotary pump, a rotary plunger pump, a screw pump, a simplex pump, a triplex pump, or a vane pump, or any combination thereof. The pump may be an axial flow pump, a radial flow pump, or a mixed flow pump. Fluid(s) may be flowed at a constant rate, at a variable rate, or at a periodic rate, or any combination thereof. The controller may be used to control the pump (e.g., the pump's flow rate, the pump's operating state, etc). One or more pumps may be used.

Fluid(s) (such as the first fluid phase and/or the second fluid phase) may be directed along the fluid flow path under generally laminar flow. Fluid(s) (again, such as the first fluid phase and/or the second fluid phase) may be directed along the fluid flow path under Stokes flow (also known as creeping flow). Fluid(s) near at least one opening may be described via Darcy's law.

The first fluid phase may comprise a liquid phase such as an oil and/or a surfactant. Many surfactants may be used including but not limited to: anionic surfactants (surfactants comprising anionic functional groups, (sulfate, sulfonate, phosphate, and carboxylates)), such as alkyl sulfates, ammonium lauryl sulfate, sodium lauryl sulfate, sodium dodecyl sulfate, alkyl-ether sulfates, sodium laureth sulfate, sodium lauryl ether sulfate, sodium myreth sulfate, dioctyle sodium sulfosuccinate, perfluorootanesulfonate, perfluorobutanesulfonate, alkyl-aryl ether phosphates, alkyl ether phosphates, carboxylates; cationic surfactants, such as octenidine dihydrochloride, cetrimonium bromide, cetylpyridinium chloride, benzalkonium chloridge, benzethonium chloride, dimethyldioctadecylammonium chloride, dioctadecyldimethylammonium bromide; zwitterionic (amphoteric) surfactants such as phospholipids, phosphatidylserine, phosphatidylethanolamine, phosphatidylcholine, and sphingomyelins; and nonionic surfactants, such as polyethylene glycol alkyl ethers; polypropylene glycol alkyl ethers, glucoside alkyl ethers, polyethylene glycol octylphenyl ethers, polyethylene glycol alkylphenyl ethers, glycerol alkyl esters, polyoxyethylene glycol sorbitan alkyl esters, sorbitan alkyl esters, cocamide mea, cocamide dea, dodecyldimethylamine oxide, block copolymers of polyethylene glycol and polypropylene glycol, poloxamers, and polyethoxylated tallow amine. One of skill in the art will appreciate that such a list of surfactants, though not exhaustive, is instructive, emphasizing the first fluid's role in droplet generation.

The second fluid phase may comprise a liquid phase such as an aqueous phase. The second fluid phase may comprise the biological sample or a portion of the biological sample. The second fluid phase may comprise reagents necessary for the chemical or biological reaction. Conversely, an optional third fluid phase may comprise reagents necessary for the chemical or biological reaction. The third fluid phase may introduced into the chamber in a manner similar to how the first fluid phase and/or the second fluid phase is introduced into the chamber.

The first fluid phase and/or second fluid phase may comprise reagents necessary for a chemical or biological reaction. Chemical or biological reactions of the method may be performed prior to droplet formation, during droplet formation, or after droplet formation. A non-limiting example of such a chemical or biological reaction may be nucleic acid amplification. Nucleic acid amplification may require reagents such as one or more primers and/or a polymerizing enzyme. Nucleic acid amplification may be via polymerase chain reaction (PCR). Nucleic acid amplification may be via isothermal amplification. Alternatively, nucleic acid amplification may be via loop mediated isothermal amplification (LAMP), nucleic acid sequence based amplification (NASBA), strand displacement amplification, multiple displacement amplification (MDA), rolling circle amplification (RCA), ligase chain reaction (LCR), helicase dependent amplification (HDA), and/or ramification amplification method (RAM). Any of the nucleic acid sequence amplification techniques may be used individually or in combination with any other nucleic acid sequence amplification technique described herein.

Droplet(s) may form within the chamber as the second fluid comes into contact with the first fluid residing in the chamber as the second fluid is flowed through at least one opening in the membrane. The first fluid phase may be immiscible with the second fluid phase (and vice versa). The droplets, or a subset thereof, may comprise the biological sample (and/or a portion thereof) and reagents necessary for the chemical or biological reaction.

Droplets of the present disclosure may take on any suitable shape. For example, the droplets may be spherical or approximately spherical. The droplets of the present disclosure may take on a shape that is not necessarily spherical (e.g. they may take on an ellipsoid shape). As referred to herein, the diameter of all such droplets will be considered as the diameter of a perfect mathematical sphere having the same volume as the non-spherical droplet. Droplets may each have a diameter of about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, or the droplets may take on a droplet size in between any two of the aforementioned values. Each of the plurality of droplets may have a droplets size from about 0.1 μm to about 200 μm, from about 1 μm to about 150 μm, and or from about 10 μm to about 100 μm. The droplets may constitute a part of an emulsion.

An average size of an individual droplet may depend on the properties (e.g. flow rate, viscosity) of one or more of the fluids, the size, configuration, or geometry of the chambers, and/or the size, configuration, or geometry of the fluid flow ports. Variations in the average size and/or shape may result from the stochastic nature of fluidic systems and/or engineering tolerances of the apparatuses and/or systems used.

Droplets may each have a size and/or shape that is at least partially dependent on the flow rate of the first fluid phase, the second fluid phase, or both, upon the second fluid phase coming into contact with the first fluid phase. The flow rate for the first fluid phase upon the second fluid phase coming into contact with the first fluid phase may be approximately 0 microliters per minute (μL/min) 0.1 μL/min, 0.2 μL/min, 0.3 μL/min, 0.4 μL/min, 0.5 μL/min, 0.6 μL/min, 0.7 μL/min, 0.8 μL/min, 0.9 μL/min, 1 μL/min, 2 μL/min, 3 μL/min, 4 μL/min, 5 μL/min, 6 μL/min, 7 μL/min, 8 μL/min, 9 μL/min, 10 μL/min, 11 μL/min, 12 μL/min, 13 μL/min, 14 μL/min, 15 μL/min, 16 μL/min, 17 μL/min, 18 μL/min, 19 μL/min, 20 μL/min, 21 μL/min, 22 μL/min, 23 μL/min, 24 μL/min, 25 μL/min, 26 μL/min, 27 μL/min, 28 μL/min, 29 μL/min, 30 μL/min, 31 μL/min, 32 μL/min, 33 μL/min, 34 μL/min, 35 μL/min, 36 μL/min, 37 μL/min, 38 μL/min, 39 μL/min, 40 μL/min, 41 μL/min, 42 μL/min, 43 μL/min, 44 μL/min, 45 μL/min, 46 μL/min, 47 μL/min, 48 μL/min, 49 μL/min, 50 μL/min, 60 μL/min, 70 μL/min, 80 μL/min, 90 μL/min, 100 μL/min, 110 μL/min, 120 μL/min, 130 μL/min, 140 μL/min, 150 μL/min, 160 μL/min, 170 μL/min, 180 μL/min, 190 μL/min, 200 μL/min, or the flow rate of the first fluid phase upon coming into contact with the second fluid phase may take on any value between any two aforementioned values. Similarly, the flow rate for the second fluid phase upon the second fluid phase coming into contact with the first fluid phase may be approximately 0 microliters per minute (μL/min) 0.1 μL/min, 0.2 μL/min, 0.3 μL/min, 0.4 μL/min, 0.5 μL/min, 0.6 μL/min, 0.7 μL/min, 0.8 μL/min, 0.9 μL/min, 1 μL/min, 2 μL/min, 3 μL/min, 4 μL/min, 5 μL/min, 6 μL/min, 7 μL/min, 8 μL/min, 9 μL/min, 10 μL/min, 11 μL/min, 12 μL/min, 13 μL/min, 14 μL/min, 15 μL/min, 16 μL/min, 17 μL/min, 18 μL/min, 19 μL/min, 20 μL/min, 21 μL/min, 22 μL/min, 23 μL/min, 24 μL/min, 25 μL/min, 26 μL/min, 27 μL/min, 28 μL/min, 29 μL/min, 30 μL/min, 31 μL/min, 32 μL/min, 33 μL/min, 34 μL/min, 35 μL/min, 36 μL/min, 37 μL/min, 38 μL/min, 39 μL/min, 40 μL/min, 41 μL/min, 42 μL/min, 43 μL/min, 44 μL/min, 45 μL/min, 46 μL/min, 47 μL/min, 48 μL/min, 49 μL/min, 50 μL/min, 60 μL/min, 70 μL/min, 80 μL/min, 90 μL/min, 100 μL/min, 110 μL/min, 120 μL/min, 130 μL/min, 140 μL/min, 150 μL/min, 160 μL/min, 170 μL/min, 180 μL/min, 190 μL/min, 200 μL/min, or the flow rate of the second fluid phase upon coming into contact with the second fluid phase may take on any value between any two aforementioned values. The flow rates of the first fluid phase and the second fluid phase may be cumulative or subtractive. The flow rates of the first fluid phase and the second fluid phase may be combined in any manner.

Other factors that may affect the size and or shape of one or more of the droplets include the pressure of the environment, the pressure difference across the membrane, the temperature of the environment, the temperature gradient, the viscosity (kinematic and dynamic) of the first fluid, the viscosity (kinematic and dynamic) of the second fluid, the viscosity difference of the first fluid and the second fluid, the size of the at least one opening of the membrane, etc.

Droplet formation and/or detachment from the membrane may be aided by a shear force perpendicular to the droplet flow direction. For example, in those embodiments in which droplets are formed by a second fluid phase coming into contact with a first fluid phase (such as one residing in a chamber) through a membrane, then a shear force perpendicular to the flow path of the second fluid phase may be used to increase the rate of droplet detachment from the membrane, such as by cross flow movement of the first fluid phase or by agitation of the membrane (such as by vibrating the apparatus or system in which the membrane resides or by moving the membrane individually or some combination thereof).

Droplet formation and/or detachment from the membrane may be further aided by decreasing the interfacial tension of a first fluid phase and a second fluid phase. Interfacial tension between the first fluid phase and the second fluid phase may be increased or decreased by introducing a third fluid phase comprising a surfactant or by incorporating a surfactant into either the first fluid phase or the second fluid phase. A surfactant may be used to decrease the interfacial tension of the first fluid phase and the second fluid phase and thereby increase droplet formation and/or detachment from the membrane. The surfactant may be of any sort described herein including but not limited to anionic surfactants, cationic surfactants, zwitterionic surfactants, and nonionic surfactants. The interfacial tension force may be reduced dynamically as a surfactant adsorbs at the interface between the first and second fluid phases. That is, the interfacial tension force may be governed at least in part by the rate of surfactant adsorption. The total reduction in interfacial tension (and thus its effects on droplet formation and/or detachment from the membrane) is a function of the specific surfactant type and concentration used.

One or more droplets may reside within a portion of the second fluid (e.g., an aqueous solution). The one or more droplets that reside within a portion of the second fluid may individually or collectively or as a part of the portion of the second fluid which contains them be completely surrounded by a first fluid (e.g., a continuous fluid, an oil, a surfactant, etc.).

A droplet of the present disclosure may be formed when a portion of a first fluid (e.g., an aqueous fluid) is substantially surrounded by a second fluid (e.g., a continuous fluid). As used herein, a portion of a first fluid is “surrounded” by a second fluid when a closed loop may be drawn around the first fluid through only the second fluid. A portion of a first fluid is “completely surrounded” by a second fluid if closed loops going through only the second fluid may be drawn around the first fluid regardless of direction. A portion of a first fluid is “substantially surrounded” by a second fluid if the loops going through only the second fluid may be drawn around the droplet depending on the direction.

After formation, a given droplet may be subjected to nucleic acid amplification. Nucleic acid amplification may be performed under conditions necessary to generate amplification product(s) from the biological sample (and/or a portion thereof) in the given droplet. As previously discussed, nucleic acid amplification techniques that the droplets and/or the constituents of the droplets may undergo include but are not limited to polymerase chain reaction, isothermal amplification, loop mediated isothermal amplification (LAMP), nucleic acid sequence based amplification (NASBA), strand displacement amplification, multiple displacement amplification (MDA), rolling circle amplification (RCA), ligase chain reaction (LCR), helicase dependent amplification (HDA), ramification amplification method (RAM), or any nucleic acid amplification technique known to one of skill in the art. The amplification product(s) may be detectable and/or detected in one or more droplets.

The method may further comprise one or more droplets from the plurality of droplet that are detectable. As such, one or more droplets from the plurality of droplets may include detectable moieties that permit detection of signals generated from the chemical or biological reactions (e.g., nucleic acid amplification reactions). For example, the detectable moieties may yield a detectable signal whose presence or absence is indicative of a presence of an amplified product. The detectable moiety may be detectable optically, biologically, chemically, radioactively, mechanically, thermally, electrically (via either passive or active electrical properties), magnetically, etc. The intensity of (e.g., the amplitude of, the frequency of, the duration of, etc.) of the detectable signal may be proportional to the amount of amplified product or it may be a function of the amount of the amount of amplified product and other. In some embodiments, where amplified product is generated of a different type of nucleic acid than the target nucleic acid initially amplified, the intensity of the detectable signal may be proportional to or a function of the amount of target nucleic acid initially amplified. For example, in the case of amplifying a target RNA via parallel reverse transcription and amplification of the DNA obtained from reverse transcription, reagents necessary for both reactions may also comprise a detectable moiety that yield a detectable signal indicative of the presence of the amplified DNA product and/or the target RNA amplified. The intensity of the detectable signal may be proportional to the amount of the amplified DNA product and/or the original target RNA amplified. The use of a detectable moiety also enables real-time amplification methods (such as real-time PCR for DNA amplification).

Detectable moieties may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, detectable moieties bind to initial reactants and changes in detectable moiety levels are used to detect amplified product. In some embodiments, detectable moieties are only detectable (or non-detectable) as nucleic acid amplification progresses. In some embodiments, an optically-active dye (e.g., a fluorescent dye) is used as a detectable moiety. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

A detectable moiety may be a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, or Lion probes.

The detectable moiety may be an RNA oligonucleotide probe. Such an oligonucleotide probe may comprise an optically-active dye (e.g., fluorescent dye) and/or a quencher positioned adjacent on the probe. Close proximity of the dye to the quencher may block the optical activity of the dye. The probe may bind to a target sequence to be amplified. Upon the breakdown of the probe with the exonuclease activity of a DNA polymerase during amplification, the quencher and dye may be separated and the free dye may regain its optical activity that can subsequently be detected.

The detectable moiety may be a molecular beacon such as a quencher linked at one end of an oligonucleotide in a hairpin conformation. At the other end of the oligonucleotide may be an optically active dye, such as a fluorescent dye. In the hairpin configuration, the optically-active dye and quencher may be brought in close proximity, such that the quencher may be capable of blocking the optical activity of the dye. Upon hybridizing with amplified product, however, the oligonucleotide may assume a linear conformation and hybridizes with a target sequence on the amplified product. Linearization of the oligonucleotide may result in separation of the optically-active dye and quencher, such that the optical activity is restored and may be detected. The sequence specificity of the molecular beacon for a target sequence on the amplified product may improve specificity and sensitivity of detection.

Other exemplary detectable moiety include but are not limited to radioactive species (e.g., 14C, 123I, 124I, 125I, 131I, Tc99m, 35S, and 3H) and enzymes capable of generating a detectable signal produce by the activity of the enzyme with its substrate or a particular substrate (e.g., alkaline phosphatase, horseradish peroxidase, I²-galactosidase, alkaline phosphatase, β-galactosidase, acetylcholinesterase, and luciferase).

In some embodiments, a detectable moiety is an enzyme that is capable of generating a detectable signal. Detectable signal may be produced by activity of the enzyme with its substrate or a particular substrate in the case the enzyme has multiple substrates. Non-limiting examples of enzymes that may be used as detectable moieties include alkaline phosphatase, horseradish peroxidase, I²-galactosidase, alkaline phosphatase, β-galactosidase, acetylcholinesterase, and luciferase.

In some embodiments, a detectable moiety may comprise a thermal liquid crystal (TLC), also known as a thermochromic liquid crystal, whose color response is a function of temperature. A TLC may comprise a material that changes its reflected color as a function of temperature when illuminated by a light of a first color (e.g., white, infrared, red, orange, yellow, green, blue, violet, ultraviolet). A detectable moiety comprising at least one TLC may reflect light (either visible or invisible) of a first wavelength at a first temperature and reflect light (either visible or invisible) of a second wavelength at a second temperature. In some embodiments, the detectable moiety may be disposed within a strip, a panel, a sheet, a plate, or a sticker. In some embodiments, the detectable moiety may be disposed within at least one droplet (in some embodiments selected from a plurality of droplets) disposed within a system such that the temperature of sample may be measured by detecting the color of the at least one droplet. Detection of the detectable moiety disposed within at least one droplet may be used to calibrate the system (e.g., prompting the controller to direct heat generation and/or cooling, prompting the controller to generate the amount droplets or the rate of droplet generation, etc.).

The method may further comprise positioning at least one droplet in sensing communication with a detector such as one capable of detecting any of the detectable moieties described herein. The detector may detect a signal from the droplet(s) that is indicative of the chemical or biological reaction or a product of the chemical or biological reaction on the biological sample.

To aid in droplet generation, droplet formation, and/or droplet guidance, the chamber may be subjected to vibration. Vibration of the chamber may be comprise one or more types of vibration including but not limited to free vibration, forced vibration, and damped vibration.

The method may further comprise monitoring a temperature of a solution comprising the plurality of droplets. The temperature may be monitored by detecting a temperature of the temperature of the solution. Temperature may be monitored using a temperature sensor. Temperature sensors

In another aspect of the present disclosure, a system for conducting a chemical or biological reaction on a biological sample may comprise a fluid flow path in fluid communication with a chamber downstream of a membrane with at least one opening and a controller programmed to (i) subject a first fluid phase to flow along the fluid path, through at least one opening in the membrane, (ii) subject a second fluid phase to flow along the fluid flow path through at least one opening in the membrane, and (iii) generate a plurality of droplets. The system may comprise a chamber on one side of the membrane (e.g., downstream of the membrane) into which the first fluid phase and the second fluid phase may flow (along the fluid flow path). Having directed the first fluid phase to flow along the fluid path through at least one opening in the membrane, the first fluid may reside within the chamber. With the first fluid residing within the chamber, the second fluid phase may be flowed through at least one opening in the membrane to the chamber. The first fluid phase may be immiscible with the second fluid phase (for example, the first fluid phase may comprise an oil and the second fluid phase may comprise an aqueous solution containing the biological sample, a portion thereof, and/or reagents necessary for the chemical or biological reaction) and thus upon the second fluid phase coming into contact with the first fluid phase within the chamber one or more droplets may be generated. At least one droplet selected from among a plurality of droplets may comprise the biological sample or a portion thereof. Alternatively or in combination, at least one droplet selected from among the plurality of droplets may comprise reagents necessary for the chemical or biological reaction.

The membrane of the system may be of any type described herein. For example, the membrane may be flexible (e.g., the membrane may comprise a material with an elastic modulus of no greater than about 100 gigapascals (GPa), 90 GPa, 80 GPa, 70 GPa, 60 GPa, 50 GPa, 40 GPa, 30 GPa, 20 GPa, 10 GPa, 9 GPa, 8 GPa, 7 GPa, 6 GPa, 5 GPa, 4 GPa, 3 GPa, 2 GPa, 1 GPa, 0.9 GPa, 0.8 GPa, 0.7 GPa, 0.6 GPa, 0.5 GPa, 0.4 GPa, 0.3 GPa, 0.2 GPa, 0.1 GPa, 90 megapascals (MPa), 80 MPa, 70 MPa, 60 MPa, 50 MPa, 40 MPa, 30 MPa, 20 MPa, 10 MPa, 9 MPa, 8 MPa, 7 MPa, 6 MPa, 5 MPa, 4 MPa, 3 MPa, 2 MPa, or 1 MPa, or the value of the elastic modulus may take a value in between any two aforementioned values). The membrane may comprise a material with an elastic modulus between about 0.1 GPa to about 5 GPa.

As previously described, the membrane may comprise one or more flexible materials including but not limited to: acetal copolymer, acetal homopolymer, acrylonitrile butadiene styrene (ABS), aluminum, bismaleimide, bismuth, boron, carbide, carbide foam, carbon, carbon foam, carbon nanofibers, cellulose, cesium, cesium iodide, copper, cyanoacrylate, ethylene chlorotrifluoroethylene (ECTFE), ethylene vinyl alcohol, furan, glass, graphite, high-density polyethylene, low-density polyethylene, maleimide, melamine, methacrylate, nylon, phenol formaldehydes, phenolics, plastarch, polyactic acid, polyamides, polyaryletherketone (PAEK), polycarbonate, polychlorotrifluoroethylene, polyepoxide, polyester, polyetheretherketone (PEEK), polyetherimide, polyethylene, polyimide, polymethyl methacrylate (PMMA), polyolefin, polypropylene, polystyrene, polysulfone, polytetrafluoroethylene (PTFE), polyurethane, polyvinyl chloride, polyvinylidene chloride, polyvinylidinefluoride (PVDF), rubidium, silicone, thermoplastic, thermoplastic elastomers, and urea-formaldehyde.

The membrane may comprise any combination of the materials described herein, variants of the materials described herein, alloys of the materials described herein, composites of the materials described herein, and/or the products of reactions involving the materials described herein so long as at least one material of the composite material used for the membrane has an elastic modulus value between about 1 MPa and 100 GPa.

The membrane may comprise a hydrophobic material. For instance, a hydrophobic coating may be applied to the membrane, such that at least a portion of the membrane (e.g., a first surface, a second surface different from the first surface, half of a first surface, etc.) may comprise a hydrophobic coating. The hydrophobicity of the membrane may an inherent property of the material comprising the membrane and/or it may arise as a function of surface features (such as microstructures) of at least a portion of the membrane. Materials that may be used to facilitate hydrophobicity on at least a portion of the membrane include but are not limited to: acrylics, amides, block copolymers, carbonates, dienes, esters, ethers, fluorocarbons, imides, olefins, styrenes vinyls, vinyl acetals, vinyl esters, vinyl eths, vinyl ketones, vinylidene chlorides, vinylpryrolidone polymers, and vinylpyridines.

Furthermore, the membrane may comprise biological materials to confer flexibility, hydrophobicity, or other desired properties (such as biocompatibility, boundary layer development, etc.). For example, the membrane may comprise a lipid bilayer. Optionally or as an alternative, the membrane or at least a portion of the membrane (e.g., an opening) may comprise at least one pore protein, such as alpha hemolysin or a variant thereof.

The membrane may intersect the fluid flow path. The fluid flow path and the membrane may intersect temporarily, periodically, permanently, and/or operatively. The intersection of the fluid flow path (as defined by a flow path vector) and the membrane (as defined by a membrane vector) may form an angle of about 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, or 180°, or the angle of intersection between the fluid flow path and the membrane may take on any value between any two aforementioned values. The intersection of the fluid flow path and the membrane may change over time such that at a first time a fluid (e.g., a first fluid, a second fluid, etc.) may flow at a first angle with respect to the membrane and at a second time the fluid may flow at a second angle with respect to the membrane. As a non-limiting example, at the first time the fluid may flow at an angle approximately perpendicular to the membrane at the second time the fluid may flow at an angle approximately parallel to the membrane. The controller may direct the fluid flow path, control the intersection of the membrane and the fluid flow path, and/or cause fluid(s) (e.g., the first fluid, the second fluid, etc.) to flow to the membrane and/or through the membrane.

In various aspects of the present disclosure, methods and systems for processing a biological sample can include heating of a solution or population of partitions or heat a solution or a population of partitions at relatively high temperature ramp rates. Relatively high temperature ramp rates can be advantageous for a number of reasons, including reduced sample processing time and reduced time of exposure of a biological sample (and any additional materials) to elevated temperatures. For example, a system can heat, or a method can include heating a solution or population of partitions, at a rate of at least about 5° C./second (“s”), at least about 10° C./s, at least about 15° C./s, at least about 20° C./s, at least about 25° C./s, at least about 30° C./s, at least about 35° C./s, at least about 40° C./s, at least about 45° C./s, at least about 50° C./s, at least about 55° C./s, at least about 60° C./s, at least about 65° C./s, at least about 70° C./s, at least about 75° C./s, at least about 80° C./s, at least about 85° C./s, at least about 90° C./s, at least about 95° C./s, at least about 100° C./s, at least about 105° C./s, at least about 110° C./s, at least about 115° C./s, at least about 120° C./s, at least about 150° C./s, at least about 200° C./s, or more. Once heating is terminated, the solution or population of partitions may cool at a cooling rate of at least about 5° C./s, at least about 10° C./s, at least about 15° C./s, at least about 20° C./s, at least about 25° C./s, at least about 30° C./s, at least about 35° C./s, at least about 40° C./s, at least about 45° C./s, at least about 50° C./s, at least about 55° C./s, at least about 60° C./s, at least about 65° C./s, at least about 70° C./s, at least about 75° C./s, at least about 80° C./s, at least about 85° C./s, at least about 90° C./s, at least about 95° C./s, at least about 100° C./s, at least about 105° C./s, at least about 110° C./s, at least about 115° C./s, at least about 120° C./s, at least about 150° C./s or more.

In various aspects, methods and systems for processing a biological sample described herein may provide for heating and/or cooling. Heating and/or cooling may be generalized (wherein a whole apparatus or system is heated and/or cooled) or heating and/or cooling may be localized (wherein at least a portion of an apparatus or a system (e.g., an individual well, a portion of a plurality of wells, a plurality of wells, a support, a channel, a chamber, etc.) is heated and/or cooled). Though both generalized heating and/or cooling (also known as a bulk heating and/or cooling) and localized heating and/or cooling may be used in any combination for methods and systems of processing a biological sample as described herein, special attention will be paid here to localized heating and/or cooling as such localized heating and/or cooling may be more efficient than bulk heating and/or cooling. However, one of skill in the art will appreciate that descriptions of localized heating and/or cooling presented here are easily applied in generalized cases.

In some examples, heating is implemented inductively to generate localized heating of partitions (e.g., droplets) and/or in some cases a solution surrounding the partitions. As described elsewhere herein, heat may be generated and/or applied by one or more heating elements. The positioning of heating elements within partitions, adjacent to partitions, and/or within a solution comprising components to-be-heated provides heat in much closer proximity to the species subject to heating. As less heat is lost to the surrounding environment with localized heating, less energy (in some cases, substantially less energy) is used for heating and more rapid heating can be achieved when compared to bulk heating at equivalent energy input.

Once heating is terminated, rapid cooling, may ensue, in some cases due to the surrounding environment being much cooler than the species (e.g., partitions, solution) being heated. As is discussed above, localized heating results in less energy needed for heating. As less energy is supplied for heating, energy transfer amounts are also lower for cooling. The relatively low temperature of a surrounding environment compared to the temperature of localized heating regions (e.g., a solution, within a population of partitions, within a partition) can rapidly transfer energy from the localized heating regions. For example, heating elements can be contained within droplets in an emulsion, such that heating is localized to the interior of the droplets. Conversely, relatively low energy is transferred to the continuous phase of the emulsion, such that the continuous phase remains at substantially the same temperature. Upon termination of heating, the large temperature gradient between the droplet interiors and the continuous phase of the emulsion can drive rapid cooling in the droplet interiors. Moreover, such cooling can also avoid inefficiencies (and, thus, slower cooling rates) associated with bulk cooling, such as inefficiencies associated with cooling bulk species that are not subject to heating.

Methods and systems of the present disclosure may be used for localized heating. In localized heating, a relatively local volume may be heated at a higher rate than a larger surrounding volume. As an alternative or in addition to, methods and systems provided herein may be used to perform bulk (e.g., 1 milliliter to 5 milliliter volume) heating. In bulk heating, an entirely of a given volume may be heated.

In various aspects, methods and system for processing a biological sample described herein can be useful for fast thermal cycling, whereby a solution of population of partitions is repeatedly heated and cooled. For example, during a nucleic acid amplification reaction, thermal cycling may repeatedly cycle the temperature of the solution or population of partitions through a denaturation temperature (e.g., in the range of 80° C.-100° C., whereby double-stranded nucleic acid separates into its single strands) and an elongation temperature (e.g., in the range of 30° C.-80° C., whereby nucleotides are incorporated into a template nucleic acid). Relatively high temperature thermal cycle times can be advantageous for a number of reasons, including reduced sample processing time. For example, a system can complete a single thermal cycle or a method can include completion of a single thermal cycle of a solution in at most about 5 minutes (“min”), at most about 4 min, at most about 3 min, at most about 2 min, at most about 1 min, at most about 45 seconds (“s”), at most about 30 s, at most about 25 s, at most about 20 s, at most about 15 s, at most about 10 s, at most about 9 s, at most about 8 s, at most about 7 s, at most about 6 s, at most about 7 s, at most about 6 s, at most about 5 s, at most about 4 s, at most about 3 s, at most about 2 s, at most about 1 s, at most about 0.5 s, at most about 0.1 s or less.

Methods and systems of the present disclosure may be used to subject a sample to one or more cycles of heating and cooling, such as at least 1, 2, 3, 4, 5, 6, 7, 8, 9, 10, 20, 30, 40, 50, 60, 70 80, 90, or 100 cycles of heating and cooling. Heating and cooling may be performed by incubating the sample at a denaturing temperature for a denaturation duration and incubating the sample at an elongation temperature at an elongation duration.

Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular biological sample analyzed, the particular source of target nucleic acid (e.g., viral particle, bacteria) in the biological sample, the reagents used, and/or the desired reaction conditions. For example, a denaturation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular biological sample analyzed, the particular source of target nucleic acid (e.g., viral particle, bacteria) in the biological sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular biological sample analyzed, the particular source of target nucleic acid (e.g., viral particle, bacteria) in the biological sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) necessary to obtain a detectable amplified product (e.g., a detectable amount of amplified DNA product that is indicative of the presence of a target RNA in a biological sample). For example, the number of cycles necessary to obtain a detectable amplified product (e.g., a detectable amount of DNA product that is indicative of the presence of a target RNA in a biological sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target RNA in a biological sample) may be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product indicative of the presence of a target nucleic acid amplified can vary depending upon the biological sample from which the target nucleic acid was obtained, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a target RNA may yield a detectable amount of amplified DNA product indicative to the presence of the target RNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions may be used.

In some embodiments, the ramping time (i.e., the time the thermal cycler takes to transition from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

In some cases, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid may be subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In some embodiments, a biological sample may be preheated prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Various aspects include a detector that detects a signal indicative of a chemical or biological reaction on a biological sample or detecting such signals. In some cases, the signals are electronic signals generated be a detector. Moreover, a chemical or biological reaction may be detected via the detection of a product (e.g., directly detecting the product itself, detecting a species indicative of the formation of product such as a reporter agent) or via one or more of its reactants (e.g., detecting the disappearance of a reactant, including the biological sample, detecting a species indicative of the disappearance of a reactant, etc.). Any suitable detector and associated detection modality can be used for detection. The particular type of detector and/or detection used may depend, for example, on the particular chemical or biological reaction, the type of any vessel in which a chemical or biological reaction takes places, whether or not a reporter agent is used, and, if a reporter agent was is used, the particular type of reporter agent. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of appropriate species after high-performance liquid chromatography separation of the species. Appropriate detectors are available for each of the example detection methods described herein, with examples that include a spectrophotometer, an imaging device (e.g., microscopes, cameras, etc.), an electrospray detector, a time-of-flight detector, an NMR detector, a conductivity detector or any combination thereof.

The controller may comprise any type described herein (see, for example, the section on “Control Systems”). The controller may comprise one or more computer processors. The controller and/or computer processor(s) thereof may be programmed to subject a first fluid phase (e.g. an oil) to flow along the fluid flow path through at least one opening in the membrane (such that the first fluid passes through the membrane into the chamber downstream of the membrane), subject a second fluid phase (e.g. a fluid phase comprising the biological sample and/or a portion thereof) to flow along the fluid flow path through at least one opening in the membrane into the chamber (the chamber at that time comprise the first fluid phase and the first fluid phase may be immiscible with the second fluid phase), and generate a plurality of droplets in the chamber. The plurality droplets may, for example, be generated upon the second fluid phase coming in contact with the first fluid phase. One or more droplets of the plurality of droplets may comprise the biological sample, a portion thereof, and/or reagents necessary for the chemical or biological reaction.

### Droplet Guidance and Isolation

In another aspect of the present disclosure, a method for facilitating a chemical or biological reaction on a biological sample comprises: providing a sample processing unit comprising a fluid flow path in fluid communication with a support (the support may comprise a plurality of wells); subjecting a plurality of droplets to flow along the fluid flow path to the support (e.g., flowing the droplets along the fluid flow path to the plurality of wells); and directing a given droplet of the plurality of droplets into an individual location of the support (such as directing the given droplet of the plurality of droplets into an individual well of the plurality of wells). The plurality droplets and/or a given droplet from the plurality of droplets of the aforementioned method may comprise the biological sample, a portion thereof, and/or reagent(s) necessary for the chemical or biological reaction.

The plurality of droplets may be generated upon a first fluid coming into contact with a second fluid (e.g., the second fluid flowing into the first fluid). One or more droplets may be generated upon the first fluid coming into contact with the second fluid. Droplets may each have a droplet size of about 0.1 micrometers (μm), 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, or the droplets may take on a droplet size in between any two of the aforementioned values. Each of the plurality of droplets may have a droplets size from about 0.1 μm to about 200 μm, from about 1 μm to about 150 μm, or from about 10 μm to about 100 μm. The droplets may constitute part of an emulsion.

Droplets may each have a droplet volume of at least about 1 nanoliter (nl), 2 nl, 3 nl, 4 nl, 5 nl, 6 nl, 7 nl, 8 nl, 9 nl, 10 nl, 20 nl, 30 nl, 40 nl, 50 nl, 60 nl, 70 nl, 80 nl, 90 nl, 100 nl, 200 nl, 300 nl, 400 nl, 500 nl, 600 nl, 700 nl, 800 nl, 900 nl, 1 microliter (μ1), 2 μl, 3 μl, 4 μl, 5 μl, 6 μl, 7 μl, 8 μl, 9 μl, 10 μl, 20 μl, 30 μl, 40 μl, 50 μl, 60 μl, 70 μl, 80 μl, 90 μl, 100 μl, 200 μl, 300 μl, 400 μl, 500 μl, 600 μl, 700 μl, 800 μl, 900 μl, milliliter (ml), 2 ml, 3 ml, 4 ml, 5 ml, 6 ml, 7 ml, 8 ml, 9 ml, or 10 ml, or the droplets may have a droplet volume between any two of the aforementioned values.

The chemical or biological reaction may be nucleic acid amplification. Such a nucleic acid amplification may accomplished via polymerase chain reaction (PCR), isothermal amplification, loop mediated isothermal amplification (LAMP), nucleic acid sequence based amplification (NASBA), strand displacement amplification, multiple displacement amplification (MDA), rolling circle amplification (RCA), ligase chain reaction (LCR), helicase dependent amplification (HDA), and/or ramification amplification method (RAM). Any of the nucleic acid sequence amplification techniques may be used individually or in combination with any other nucleic acid sequence amplification technique described herein. Reagents necessary for the chemical or biological reaction (such as for nucleic acid amplification) may comprise one or more primers and polymerizing enzyme.

The method may further comprise subjecting the plurality of droplets to nucleic acid amplification under conditions necessary and/or sufficient to generate amplification product(s) from at least a portion (e.g., a subset) of the biological sample and/or a portion thereof within each of the plurality of droplets with such contents (e.g., comprising a biological sample and/or a portion thereof). The amplification product(s) of at least a portion of the plurality of droplets may be detectable (e.g., detectable optically, biologically, chemically, radioactively, mechanically, thermally, electrically (via either passive or active electrical properties), magnetically, etc.). The method may further comprise detecting the amplification product(s) in at least a subset of the plurality of droplets. Detection may be via any of the techniques described herein or known in the art.

To detect the amplification product(s) of at least a portion of the plurality of droplets may require that the method further comprise positioning at least a portion of the plurality of droplets in sensing communication with a detector such as one capable of detecting any of the detectable moieties described herein. The detector may detect a signal from the droplet(s) that is indicative of the chemical or biological reaction or a product of the chemical or biological reaction on the biological sample.

The support may comprise one or more wells. Of those one or more wells, at least one individual well may comprise a hygroscopic material. Among the possible hygroscopic materials that may comprise at least one individual well from among the one or more wells of the support include but are not limited to: cellulose fibers (e.g., cotton, paper, etc.), sugar, caramel, honey, glycerol, ethanol, methanol, sulfuric acid, salts (e.g., NaCl), polysaccharides. Several polymers may also be used for their hygroscopic properties including but not limited to acrylonitrile butadiene styrene (ABS), nylon, polycarbonate, polyethylene, poly(methyl methacrylate), and polystyrene. Though other, stronger, hygroscopic materials may also be used (such as calcium chloride, potassium hydroxide, sodium hydroxide, zinc chloride, etc.), one of skill in the art should exercise caution depending on a confluence of factors including the biological sample used, the chemical or biological reaction to be achieved, the size of the droplet(s), the water content of the droplet(s), etc. as these more strongly hygroscopic materials may so readily absorb water that they readily dissolve (e.g., deliquescence). Desiccants may be used as the hygroscopic material provided they do not adversely affect the chemical or biological reaction the biological sample is meant to be subjected to.

The support may be of any shape and/or dimension suitable for holding an aqueous solution comprising a biological sample, a portion thereof, and/or reagents necessary for a chemical or biological reaction. The support may have a shape and/or dimension suitable for holding at least one droplet from a plurality of droplets. The support may comprise a plurality of wells. At least a portion of the wells may have a shape and/or dimension suitable for holding at least one droplet from a plurality of droplets. For example, the support may comprise one or more wells that are conic, cubic, or cylindrical in shape. It should be appreciated that the one or more wells of the plurality of wells of the support may have a shape that is a combination of other shapes (e.g., half a circle and half a square).

The support may be dimensioned to hold a first fluid volume of at least about 1 nanoliter (nl), 2 nl, 3 nl, 4 nl, 5 nl, 6 nl, 7 nl, 8 nl, 9 nl, 10 nl, 20 nl, 30 nl, 40 nl, 50 nl, 60 nl, 70 nl, 80 nl, 90 nl, 0.1 microliters (μl), 0.5 μl, 1 μl, 1.5 μl, 2 μl, 2.5 μl, 3 μl, 3.5 μl, 4 μl, 4.5 μl, 5 μl, 5.5 μl, 6 μl, 6.5 μl, 7 μl, 7.5 μl, 8 μl, 8.5 μl, 9 μl, 9.5 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 110 μl, 120 μl, 130 μl, 140 μl, 150 μl, 160 μl, 170 μl, 180 μl, 190 μl, 200 μl, 300 μl, 400 μl, 500 μl, 600 μl, 700 μl, 800 μl, 900 μl, 1000 μl, 2 ml, 3 ml, 4 ml, 5 ml, 6 ml, 7 ml, 8 ml, 9 ml, or more, or the support may be dimensioned to hold a first fluid volume about equal to a value between any two aforementioned values.

The support comprise a plurality of wells of which at least one may be dimensioned to hold a first fluid volume of at least about 1 nl, 2 nl, 3 nl, 4 nl, 5 nl, 6 nl, 7 nl, 8 nl, 9 nl, 10 nl, 20 nl, 30 nl, 40 nl, 50 nl, 60 nl, 70 nl, 80 nl, 90 nl, 0.1 μl, 0.5 μl, 1 μl, 1.5 μl, 2 μl, 2.5 μl, 3 μl, 3.5 μl, 4 μl, 4.5 μl, 5 μl, 5.5 μl, 6 μl, 6.5 μl, 7 μl, 7.5 μl, 8 μl, 8.5 μl, 9 μl, 9.5 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 110 μl, 120 μl, 130 μl, 140 μl, 150 μl, 160 μl, 170 μl, 180 μl, 190 μl, 200 μl, 300 μl, 400 μl, 500 μl, 600 μl, 700 μl, 800 μl, 900 μl, 1000 μl, 2 ml, 3 ml, 4 ml, 5 ml, 6 ml, 7 ml, 8 ml, 9 ml, or more, or the at least one well from the plurality of wells from the support may be dimensioned to hold a first fluid volume about equal to a value between any two aforementioned values.

The support may comprise one or more first fluid flow ports. At least one of the one or more first fluid ports may be in fluid communication with the first fluid volume. For example, an aqueous solution comprising a biological sample, a portion thereof, and/or reagents necessary for a chemical or biological reaction may flow in and/or out of the first chamber through the one or more first fluid flow ports. The one or more first fluid flow ports may be of the same or of different shapes, and they may be of the same or different dimensions. For example, each of the one or more first fluid flow ports may independently have a diameter of no more than about 1 mm, no more than about 800 μm, no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, no more than about 5 μm, no more than about 4 μm, no more than about 3 μm, no more than about 2 μm, no more than about 1 μm, or less or each of the one or more first fluid flow ports may independently have a diameter about equal to a value between any two aforementioned values. In some embodiments, each of the one or more first fluid flow ports may independently have a diameter of at least about 1 μm, at least about 2 μm, at least about 3 μm, at least about 4 μm, at least about 5 μm, at least about 10 μm, at least about 15 μm, at least about 20 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 800 μm, or more, or each of the one or more first fluid flow ports may independently have a diameter about equal to a value between any two aforementioned values. In some embodiments, each of the one or more first fluid flow ports may have a diameter that is greater than a cross-section of the at least one droplet.

The method may further comprise directing one or more droplets from the plurality of droplets along either a first fluid flow path or a second fluid flow path, or both, wherein the first fluid flow path and the second fluid flow path are in fluid communication with the support. The support may be of any type described herein. For example, the support may comprise a plurality of wells. That plurality of wells may comprise at least one individual well with a first opening adjacent to the first fluid flow path. Alternatively or in combination, at least one individual well may comprise a second opening adjacent to the second fluid flow path.

One or more droplets from the plurality of droplets may be caused to flow along the first fluid flow path or the second fluid flow path to the plurality of wells. A given droplet from the plurality of droplets may comprise the biological sample, a portion thereof, and/or reagents necessary for the chemical or biological reaction. Upon flowing along a fluid flow path (e.g. the first fluid flow path, the second fluid flow path, etc.) one or more droplets from the plurality of droplets may be direct into an individual well of the plurality of wells through an opening in individual well selected from the plurality of wells. An individual well may comprise a first opening in fluid communication with the first fluid flow path. The individual well may further comprise a second opening in fluid communication with the second fluid flow path. A first fluid phase (e.g., an oil, a surfactant, a continuous fluid, any combination thereof, etc.) in the first fluid flow path and a second fluid phase (e.g., an aqueous solution, a solution comprising the biological sample, a portion thereof, reagents necessary for the chemical or biological reaction, any combination thereof, etc.) in the second fluid flow path may be provided. The first fluid phase may have a first fluidic property (e.g., density, viscosity (kinematic, dynamics, etc.), temperature, pressure, specific volume, specific weight, specific gravity, etc.) and the second fluid phase may have a second fluidic property (e.g., density, viscosity (kinematic, dynamics, etc.), temperature, pressure, specific volume, specific weight, specific gravity, etc.) that differs from the first fluidic property of the first fluid phase. For example, the first fluid phase may comprise a fluid with a first density greater than the density of the individual droplet from the plurality of droplets and the second fluid phase may comprise a fluid with a second density less than the density of the individual droplet from the plurality of droplets. As such, the individual droplet from the plurality of droplets may be retained within the individual well of the plurality of wells. One of skill in the art will appreciate that other such combinations of first fluidic properties and second fluidic properties may be used to retain the individual droplet within the individual well such as a first pressure and a second pressure, a first flow rate and a second flow rate, etc.

An individual droplet may be retained within an individual well by a fluid phase adjacent to the individual well. Such a fluid phase adjacent to the individual well may seal the individual droplet with the individual well.

One or more droplets may be retained within an individual well for any period of time. One or more droplets from the plurality of droplets may be retained within one or more individual wells in order for the chemical or biological reaction to occur, or one or more droplets from the plurality of droplets may be retained within one or more individual wells in order to detect that a chemical or biological reaction has occurred, or one or more droplets from the plurality of droplets may be retained within one or more individual wells in order to determine the extent to which a chemical or biological reaction has occurred (e.g., how much product(s) have been produced, how fast a reaction has occurred, etc.).

To aid in droplet guidance, the support may be subjected to vibration. Vibration of the support may be comprise one or more types of vibration including but not limited to free vibration, forced vibration, and damped vibration.

In another aspect of the present disclosure a system for conducting a chemical or biological reaction on a biological sample may comprising a sample processing unit, a fluid flow path in fluid communication with a support, the support comprising a plurality of wells, and a controller. An individual well of the plurality of wells may comprise a hygroscopic material that directs a given droplet from the plurality of droplets to the individual well.

The support may comprise one or more individual wells selected from the plurality of wells that comprise a hygroscopic material adapted to direct one or more droplets from the plurality of droplets to the one or more individual wells. The support may include at least 1 well, 2 wells, 3 wells, 4 wells, 5 wells, 10 wells, 100 wells, 200 wells, 300 wells, 400 wells, 500 wells, 1,000 wells, 10,000 wells, 100,000 wells, or 1,000,000 wells. The wells may at least partially or substantially protrude into the support.

The controller may comprise one or more computer processors. The controller and/or the one or more computer processes may individually or collectively be programmed to subject the plurality of droplets to flow along the fluid flow path or direct a given droplet of the plurality of droplets into an individual well. A given droplet from the plurality of droplets or the plurality of droplets themselves may comprise the biological sample, a portion thereof, and/or the reagents necessary for the chemical or biological reaction.

In another aspect of the present disclosure an apparatus for facilitating a chemical or biological reaction on a biological sample may comprise a support. The support may comprise a plurality of wells. The plurality of wells may comprise at least one individual well that comprises a hygroscopic material that either directs a given droplet from a plurality of droplets to the at least one individual well or retains the given droplet in the at least one individual well during the chemical or biological reaction, or both.

In another aspect of the present disclosure a method for facilitating a chemical or biological reaction on a biological sample may comprise providing a sample processing unit comprising a first fluid flow path and a second fluid flow path in fluid communication with a support. The support may be of any type described herein. For example, the support may comprise a plurality of wells. That plurality of wells may comprise at least one individual well with a first opening adjacent to the first fluid flow path. Alternatively or in combination, at least one individual well may comprise a second opening adjacent to the second fluid flow path.

The method may further comprise subjecting the plurality of droplets to flow along the first fluid flow path or the second fluid flow path to the plurality of wells. A given droplet from the plurality of droplets may comprise the biological sample, a portion thereof, and/or reagents necessary for the chemical or biological reaction.

The method may also further comprise directing the given droplet of the plurality of droplets from the first fluid flow path or the second fluid flow path into the individual well of the plurality of wells through the first opening or second opening.

The method may further comprise providing a first fluid phase in the first fluid flow path and a second fluid phase in the second fluid flow path. The first fluid phase may have a first fluidic property (e.g., density, viscosity (kinematic, dynamics, etc.), temperature, pressure, specific volume, specific weight, specific gravity, etc.) and the second fluid phase may have a second fluidic property (e.g., density, viscosity (kinematic, dynamics, etc.), temperature, pressure, specific volume, specific weight, specific gravity, etc.) that differs from the first fluidic property of the first fluid phase. For example, the first fluid phase may comprise a fluid with a first density greater than the density of the individual droplet from the plurality of droplets and the second fluid phase may comprise a fluid with a second density less than the density of the individual droplet from the plurality of droplets. As such, the individual droplet from the plurality of droplets may be retained within the individual well of the plurality of wells.

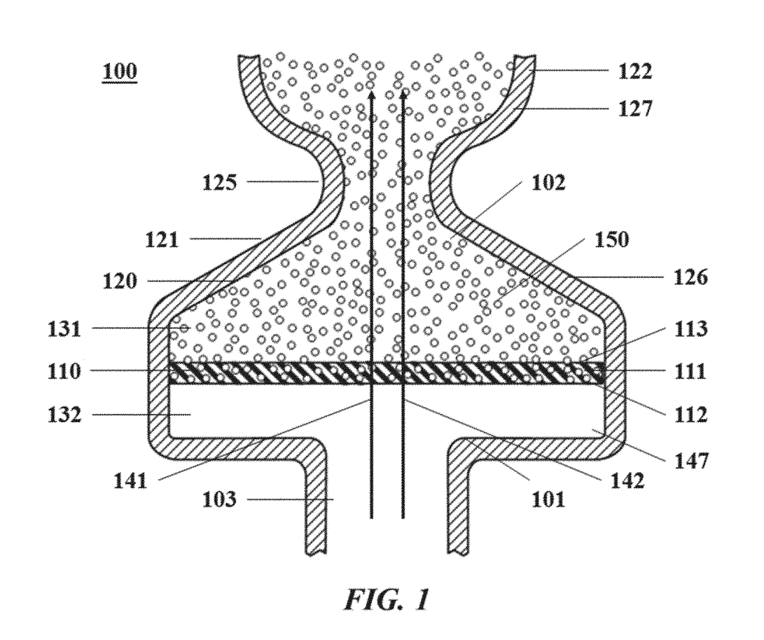

FIG. 1 shows a cross-sectional view of a droplet generation apparatus 100. The droplet generation apparatus 100 may comprise a first chamber 101 (also referred to herein as an “antechamber,” a “preparatory chamber,” and an “initial chamber”) and a second chamber 102 (also referred to herein simply as “the chamber”) separated by a membrane 110. From the first chamber 101 to the second chamber 102 extends a first flow path 141 and a second flow path 142 along which fluid(s) may flow (e.g., a first fluid phase 131, a second fluid phase 132, etc.). The first flow path 141 and the second flow path 142 may intersect the membrane 110 at any angle either individually or collectively. For example, in some embodiments the first flow path 141 and the second flow path each intersect the membrane 110 at the normal to the interface (e.g., 90° to the tangent of the surface at the point of intersection. In general, the first flow path 141 and the second flow path 142 may either individually or in combination intersect the membrane at an angle of no more than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or 90° from the normal defined by the tangent of the interface at the membrane 110 where the first flow path 141 or the second flow path 142, or both, intersect the membrane 110. The first flow path 141 and/or the second fluid flow path 142 may intersect the membrane 110 temporarily, periodically, permanently, and/or operatively. The intersection of the first flow path 141 and/or the second flow path 142 and the membrane 110 may change over time such that at a first time a fluid (e.g., a first fluid phase 131, a second fluid phase 132, etc.) may flow at a first angle with respect to the membrane 110 and at a second time the fluid may flow at a second angle with respect to the membrane 110. As a non-limiting example, at the first time the fluid may flow at an angle approximately perpendicular to the membrane 110 and at a second time the fluid may flow at an angle approximately parallel to the membrane 110.

The droplet generation apparatus 100 may comprise an interior surface defining a vessel 120 that holds the first fluid phase 131, the second fluid phase 132, both, or neither. The vessel may be of any sort described herein. For example, the vessel 120 may be a reaction vessel (e.g., a PCR tube) that receives a solution comprising a biological sample. The vessel 120 may be of various sizes, shapes, weights, and configurations. In some embodiments, the vessel 120 is round or oval tubular shaped. In some embodiments, the vessel 120 is rectangular, square, diamond, circular, elliptical, or triangular shaped. The vessel 120 may be regularly shaped or irregularly shaped. For example, a vessel 120 may be a chamber, a tube, a well, a capillary tube, a cartridge, a cuvette, a centrifuge tube, a pipette tip. In some embodiments, the vessel 120 has a surface area to volume ratio of at least 100 mm⁻¹, 200 mm⁻¹, 300 mm⁻¹, 350 mm⁻¹, 400 mm⁻¹, 450 mm⁻¹, 500 mm⁻¹, 1×10³ mm⁻¹, 1×10⁴ mm⁻¹ 1×10⁵ mm⁻¹, 1×10⁶ mm⁻¹ 1×10⁷ mm⁻¹ 1×10⁸ mm⁻¹ 1×10⁹ mm⁻¹ 1×10¹⁰ mm⁻¹, 1×10¹¹ mm⁻¹, 1×10¹² mm⁻¹, 1×10¹³ mm⁻¹, 1×10¹⁴ mm⁻¹, 1×10¹⁵ mm⁻¹ or more.

In some embodiments, a vessel 120 is part of an array of vessels. An array of vessels may be integral with the droplet generation apparatus 100. An array of vessels may comprise multiple droplet generation apparatuses 100. An array of vessels may comprise a modulate combination of vessels (e.g., a first vessel 120 may be integral with a droplet generation apparatus 100 and a second vessel (not illustrated) may be coupled to the droplet generation apparatus 100). An array of vessels may be used for automating methods and/or simultaneously processing multiple samples. For example, a vessel 120 may be a well of a microwell plate comprised of a number of wells. An array of vessels may comprise any appropriate number of vessels 120. For example, an array may comprise at least 2, 4, 6, 8, 10, 12, 14, 16, 18, 20, 25, 35, 48, 96, 144, 288, 384, or more vessels 120. A vessel 120 part of an array of vessels may also be individually addressable by a fluid handling device, such that the fluid handling device can correctly identify a vessel 120 and dispense appropriate fluid materials into the vessel 120. Fluid handling devices may be useful in automating the addition of fluid materials to the vessel 120.

The first chamber 101 may comprise an entrance region 103 through which a first fluid phase 131 or a second fluid phase 132 or both are flowed. The first chamber 101 may further comprise a first pressure reducing region 147 downstream of the entrance region 103 and upstream of the membrane 110 so that a fluid flowing from the entrance region 103 to the first chamber 101 may experience a pressure drop along the pressure reducing region 147.

The pressure reducing region 147 of the first chamber 101 may comprise a widening of a channel, a cross-sectional area change along the length of the pressure reducing region, a diffuser, etc. The pressure reducing region 147 of the first chamber 101 may be figured such that flow across the membrane 110 is substantially constant across at least a first portion of the membrane. For example, the fluid flow across the membrane 110 may have a constant velocity for at least about 1%, 2%, 3%, 4%, 5%, 6%, 7%, 8%, 9%, 10%, 20%, 30%, 40%, 50%, 60%, 70%, 80%, 90%, 95%, or 100% of the cross-section of the membrane 110 that intersects the first flow path 141 or the second flow path 142 or both. Similarly, the fluid flow across the membrane 110 may have a constant flow rate for at least about 1%, 2%, 3%, 4%, 5%, 6%, 7%, 8%, 9%, 10%, 20%, 30%, 40%, 50%, 60%, 70%, 80%, 90%, 95%, or 100% of the cross-section of the membrane 110 that intersects the first flow path 141 or the second flow path 142 or both.

The pressure reducing region 147 may reduce the flow rate of a fluid passing along its length by at least about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure reducing region 147 may reduce the flow rate of a fluid passing along its length by any amount in between any two aforementioned values. The pressure reducing region 147 may reduce the flow rate of a fluid passing along its length by no more than about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure reducing region 147 may reduce the flow rate of a fluid passing along its length by any amount in between any two aforementioned values. The pressure reducing region 147 may reduce the pressure (e.g., an average pressure, a local pressure, a pressure measured by a sensor, etc.) by at least about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure reducing region 147 may reduce the pressure of a fluid passing along its length by any amount in between any two aforementioned values. The pressure reducing region 147 may reduce the pressure (e.g., an average pressure, a local pressure, a pressure measured by a sensor, etc.) by no more than about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure reducing region 147 may reduce the pressure of a fluid passing along its length by any amount in between any two aforementioned values.

The second chamber 102 may comprise a first pressure increasing region 126 that increases may increase the flow rate of a fluid passing along its length by at least about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure increasing region 126 may reduce the flow rate of a fluid passing along its length by any amount in between any two aforementioned values. The pressure increasing region 126 may reduce the flow rate of a fluid passing along its length by no more than about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure increasing region 126 may reduce the flow rate of a fluid passing along its length by any amount in between any two aforementioned values. The pressure increasing region 126 may reduce the pressure (e.g., an average pressure, a local pressure, a pressure measured by a sensor, etc.) by at least about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure increasing region 126 may reduce the pressure of a fluid passing along its length by any amount in between any two aforementioned values. The pressure increasing region 126 may reduce the pressure (e.g., an average pressure, a local pressure, a pressure measured by a sensor, etc.) by no more than about 1%, 2%, 3%, 4%, 5%, 6%, 7%, 8%, 9%, 10%, 20%, 30%, 40%, 50%, 60%, 70%, 80%, 90%, 100%, 125%, 150%, 175%, 200%, 250%, 300%, 400%, 500%, 600%, 700%, 800%, 900%, 1000%, 2000%, 3000%, 4000%, 5000%, 6000%, 7000%, 8000%, 9000%, or 10000%, or the pressure increasing region 126 may reduce the pressure of a fluid passing along its length by any amount in between any two aforementioned values.

The second chamber 102 may further comprise a first pressure reducing region 127. The first pressure reducing region 127 of the second chamber 102 may be of a type similar to the pressure reducing region 147 of the first chamber 101. Indeed, the first pressure reducing region 127 of the second chamber 102 may be of any type described herein. Moreover, though the illustrated embodiment of FIG. 1 has a first chamber 101 comprising a single pressure reducing region 147 and a second chamber 102 comprising a first pressure increasing region 126 and a first pressure reducing region 127, the first 101 and second chambers 102 may individually or collectively comprise any number of pressure increasing regions and/or pressure reducing regions. Moreover, the first chamber 101 and second chamber 102 may individually or collectively comprise any number of pressure increasing regions and/or pressure reducing regions in any combination (e.g., a first pressure reducing region in the first chamber followed by (that is, downstream of) a further pressure reducing region of the second chamber followed by a pressure increasing region in the second chamber, etc.).

The second chamber 102 may further comprise a narrowed region 125. The narrowed region 125 may, in some instances, be accompanied by a taper 121 that reduces the initial cross-sectional area and/or shape (being here defined by the area and/or shape of the second chamber 102 just downstream of the membrane 110) of the second chamber 102 to the cross-sectional area and/or shape of the narrowed region 125. An additional taper, also referred to here as an expanding region 122, may be used to increase the cross-sectional area and/or shape of the chamber from the narrowed region 125 to a region downstream.

The narrowed region 125 may serve a number of roles. In some cases, the narrowed region 125 provides a portion of the droplet generation apparatus 100 that a user or a machine may use to comfortable grab, hold, and/or manipulate the droplet generation apparatus 100. In some cases, the narrowed region 125 provides a portion of the droplet generation apparatus 100 that may couple to a droplet generation system. In some cases, the narrowed region 125 comprises a pressure increasing region. Though the above descriptions are stated with respect to the second chamber 102, one of skill in the art will appreciate that either the first chamber 101 or the second chamber 102 or both or neither may comprise a narrowed region 125. The narrowed region 125 of either the first chamber 101 or the second chamber 102 or both may lie downstream of the membrane 110, upstream of the membrane 110, or any combination thereof. Furthermore, though only a single narrowed region 125 is illustrated in FIG. 1 any number of narrowed regions may be used across any number of chambers in any combination. In some embodiments, the narrowed region 125 has the membrane 110 disposed there within.

The membrane 110 may be of any type described herein. For example, the membrane 110 may be flexible, such that the membrane 110 comprises a material an elastic modulus between about 0.1 GPa to about 5 GPa. Such materials that may comprise the membrane 110 of this or any embodiment include but are not limited to: acetal copolymer, acetal homopolymer, acrylonitrile butadiene styrene (ABS), aluminum, bismaleimide, bismuth, boron, carbide, carbide foam, carbon, carbon foam, carbon nanofibers, cellulose, cesium, cesium iodide, copper, cyanoacrylate, ethylene chlorotrifluoroethylene (ECTFE), ethylene vinyl alcohol, furan, glass, graphite, high-density polyethylene, low-density polyethylene, maleimide, melamine, methacrylate, nylon, phenol formaldehydes, phenolics, plastarch, polyactic acid, polyamides, polyaryletherketone (PAEK), polycarbonate, polychlorotrifluoroethylene, polyepoxide, polyester, polyetheretherketone (PEEK), polyetherimide, polyethylene, polyimide, polymethyl methacrylate (PMMA), polyolefin, polypropylene, polystyrene, polysulfone, polytetrafluoroethylene (PTFE), polyurethane, polyvinyl chloride, polyvinylidene chloride, polyvinylidinefluoride (PVDF), rubidium, silicone, thermoplastic, thermoplastic elastomers, and urea-formaldehyde. Alloys and/or composites of the aforementioned materials may also be used. One or more portions of the membrane 110 may comprise at least a first material and a second material, the second material having greater flexibility than the first material such that the combined flexibility of the membrane 110 is greater than the flexibility of the first material. Composite materials (a material comprising two or more constituent materials of different physical and/or chemical properties) may be used for the membrane 110, so long as at least one material of the composite material used for the membrane 110 has an elastic modulus value between about 1 MPa and 100 GPa. The membrane 110 may comprise any combination of the materials described herein, variants of the materials described herein, alloys of the materials described herein, and/or the products of reactions involving the materials described herein.

The flexibility of the membrane 110 may be due at least in part to the structure and/or geometrical configuration of the membrane 110. For example, the membrane 110 may comprise a thin membrane 110 whose ratio of thickness to its diameter (diameter here referring to the diameter of a perfect circle whose cross-sectional area is equal to the cross-sectional area of the membrane 110) may be no greater than about 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0009, 0.0008, 0.0007, 0.0006, 0.0005, 0.0004, 0.0003, 0.0002, 0.0001, 0.00009, 0.00008, 0.00007, 0.00006, 0.00005, 0.00004, 0.00003, 0.00002, 0.00001, 0.000009, 0.000008, 0.000007, 0.000006, 0.000005, 0.000004, 0.000003, 0.000002, 0.000001, 0.0000009, 0.0000008, 0.0000007, 0.0000006, 0.0000005, 0.0000004, 0.0000003, 0.0000002, 0.0000001, 0.00000005, or the ratio of the thickness of the membrane 110 to its diameter may take on any value between any two of the aforementioned values. Moreover, the membrane 110 may have a thickness of no more than about 5 nanometers (nm), 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 micrometer (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeter (mm), 2 mm, 3 mm, 4 mm, 5 mm, 6 mm, 7 mm, 8 mm, 9 mm, 1 centimeter (cm), 2 cm, 3 cm, 4 cm, 5 cm, 6 cm, 7 cm, 8 cm, 9 cm, 10 cm, or the thickness of the membrane may take on any value between any two aforementioned values.

The flexibility of the membrane 110 may be due at least in part to one or more holes extending at least partially from a first side of the membrane 110 toward a second side of the membrane. The membrane 110 may comprise divots along a first surface of the membrane 110 or along a second surface of the membrane 110 or both or channels extending along one surface of the membrane 110. The membrane 110 may comprise any combination of one or more holes extending partially through the membrane 110, such as one set of divots residing on the first surface of the membrane and another set of divots residing on the second surface of the membrane. In some embodiments, the membrane 110 comprises a combination of one or more holes extending partially from the first side of the membrane 110 toward the second side of the membrane 110 and one or more holes extending fully from the first side of the membrane 110 to the second side of the membrane 110.

The membrane 110 may comprise at least one opening 111 through the membrane 110. The at least one opening 111—in some embodiments the at least one opening 111 is chosen from a plurality of openings—may take on any shape including but not limited to a circle, an oval, an ellipse, a triangle, a square, a pentagon, a hexagon, a polygon, or any profile that may be described as the sum of any number of sine and cosine functions. Opening(s) 111 within the membrane 110 may have a diameter no greater than about 1 mm, 900 micrometers (μm), 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 900 nanometers (nm), 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 9 nm, 8 nm, 7 nm, 6 nm, 5 nm, 4 nm, 3 nm, 2 nm, 1 nm, or the size of the opening(s) 111 of the membrane 110 may take on a value in between any two of the aforementioned values. The opening(s) 111 within the membrane 110 may have a diameter from approximately 1 μm to about 50 μm. Opening(s) 111 may have a uniform cross-sectional area and/or shape such that the cross-sectional area and/or shape of a first side 112 of the opening 111 is equivalent to the cross-sectional area and/or shape of a second side 113 of the opening 111. In some embodiments, opening(s) 111 in the membrane 110 may a cross-sectional area and/or shape that varies along their length from the first side 112 of the membrane to the second side 113 of the opening 111 (e.g., the cross-sectional area may increase from one side to another, the cross-sectional area may decrease from one side to another, etc.).

The membrane 110 may comprise any number of openings 111 through the membrane 110. For example, the membrane 110 may comprise at least 1 opening, 2 openings, 3 openings, 4 openings, 5 openings, 6 openings, 7 openings, 8 openings, 9 openings, 10 openings, 20 openings, 30 openings, 40 openings, 50 openings, 60 openings, 70 openings, 80 openings, 90 openings, 100 openings, 200 openings, 300 openings, 400 openings, 500 openings, 600 openings, 700 openings, 800 openings, 900 openings, 1,000 openings, 2,000 openings, 3,000 openings, 4,000 openings, 5,000 openings, 6,000 openings, 7,000 openings, 8,000 openings, 9,000 openings, 10,000 openings, 20,000 openings, 30,000 openings, 40,000 openings, 50,000 openings, 60,000 openings, 70,000 openings, 80,000 openings, 90,000 openings, 100,000 openings, 200,000 openings, 300,000 openings, 400,000 openings, 500,000 openings, 600,000 openings, 700,000 openings, 800,000 openings, 900,000 openings, 1,000,000 openings, 2,000,000 openings, 3,000,000 openings, 4,000,000 openings, 5,000,000 openings, 6,000,000 openings, 7,000,000 openings, 8,000,000 openings, 9,000,000 openings, 10,000,000 openings, or the number of openings 11 through the membrane 110 may take on a value between any two of the aforementioned values.

At least one opening 111 of the membrane 110 may permit fluid(s) to flow along in one direction only (in the direction of the chamber 102, for instance). Unidirectional flow along the at least one opening 111 of the membrane 110 may be aided at least in part by a valve as described elsewhere within.

For those embodiments comprising at least two openings (such as FIG. 1), the openings may be spaced apart from each other in any pattern including but not limited to a linear pattern, a grid-like pattern, a radial-like pattern, a spiral-like pattern, a Poisson-distribution-based pattern, etc. The spacing(s) between an opening and its neighboring opening(s) may be uniform or it may vary. Spacing between an opening and its nearest neighbor may be about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1 mm, or the spacing between an opening and its nearest neighbor may take on a value between any two of the aforementioned values. The distribution of openings may be symmetric or asymmetric.

A hydrophobic coating (not illustrated) may be applied to the membrane 110, such that at least a portion of the membrane 110 (e.g., a first surface, a second surface different from the first surface, half of a first surface, at least one opening, the region(s) near opening(s), etc.) may comprise a hydrophobic coating. The membrane 110 itself may be hydrophobic and/or comprise a hydrophobic material. Such hydrophobicity may an inherent property of the material comprising the membrane 110 and/or it may arise as a function of surface features (such as microstructures) of at least a portion of the membrane. Materials that may be used to facilitate hydrophobicity on at least a portion of the membrane include but are not limited to: acrylics, amides, block copolymers, carbonates, dienes, esters, ethers, fluorocarbons, imides, olefins, styrenes vinyls, vinyl acetals, vinyl esters, vinyl eths, vinyl ketones, vinylidene chlorides, vinylpryrolidone polymers, and vinylpyridines.

The membrane 110 may comprise one or more biological materials to confer flexibility, hydrophobicity, or other desired properties (such as biocompatibility, boundary layer development, etc.). For example, the membrane 110 may comprise a lipid bilayer. Optionally or as an alternative, the membrane 110 or at least a portion of the membrane (e.g., an opening) may comprise at least one pore protein, such as alpha hemolysin or a variant thereof.

In some embodiments the membrane 110 may have a portion that is hydrophobic. Hydrophobic membrane embodiments may be hydrophobic as a result of microsurface structures disposed on the membrane 110 or the membrane 110 may be hydrophobic because the membrane comprises a hydrophobic material. In some embodiments, the membrane 110 includes a lipid bilayer. In some embodiments, the at least one opening in the membrane permits fluid flow only along a directing leading to the chamber. In some embodiments, the at least one opening includes a one-way valve. The one-way valve of some embodiments is actively controlled. The one-way valve of some embodiments is passively controlled. In some embodiments the at least one opening includes a port protein. The pore protein of some embodiments comprises alpha hemolysin or a variant thereof.

At a given time the first chamber 101 may contain the first fluid phase 131 (shown illustrated in the second chamber 102) or the second fluid phase 132 (as illustrated in FIG. 1) both or neither. The first fluid phase 131 may comprise a continuous fluid phase such as an oil (e.g., hydrocarbons, silicon oils, fluorine-containing oils (e.g., fluorocarbon oils), organic solvents etc.). The second fluid phase 132 may comprise an aqueous fluid phase, such as one, for example, that comprises the biological sample or a portion thereof. The first fluid phase 131 and the second fluid phase 132 may be immiscible.

In some embodiments, the first fluid phase 131 is directed from the entrance region 103 through the first chamber 101 along the first flow path 141 through the membrane 110 (via at least one opening 111, traversing from a first side 112 of the opening 111 to a second side 113 of the opening 111) into the second chamber 102. The first fluid phase 131 may be held in the second chamber 102 of the vessel 120. In some embodiments, the second fluid phase 132 is directed from the entrance region 103 through the first chamber 101 along the second flow path 141 through the membrane (via at least one opening 111, traversing from a first side 112 of the opening 111 to a second side 113 of the opening 111) into the second chamber 102 where, in some instances, it may come into contact with the first fluid phase 131 residing there within. In those cases in which the second fluid phase 132 comes into contact with the first fluid phase 131 one or more droplets 150 may be generated upon the second fluid phase 132 coming into contact with the first fluid phase 131.

The droplets 150 of any embodiment may comprise one or more droplets 150. The droplets 150 of some embodiments comprise a plurality of droplets, and each of the plurality of droplets may comprise the biological sample or a portion thereof.

The droplets 150 may have a size that is at least partially dependent on a flow rate of a first fluid phase 131 or the droplets 150 may have a size that is at least partially dependent on a flow rate of a second fluid phase 132 or the droplets 150 may have a size that is at least partially dependent on the net flow rate with respect to the first fluid phase 131 and the second fluid phase 132. For example, for those embodiments in which the first fluid phase 131 is held within the chamber 102 as the second fluid phase 132 is introduced to the chamber 102 (for example, through at least one opening 111 of the membrane 110), droplets 150 of a first size may form as the second fluid phase 132 comes into contact with the first fluid phase 131 (for example, because the first fluid phase 131 and the second fluid phase 132 are immiscible) if the second fluid phase 132 is flowed at a first flow rate and droplets 150 of a second size may form if the second fluid phase 132 is flow at a second flow rate. If, for example, the first flow rate is greater than the second flow rate, the droplets 150 of the first size of the previous example would be larger than the droplets 150 of the second size. That is, at least in some embodiments, the larger the flow rate, the larger the droplets 150 produced upon the second fluid phase 132 coming into contact with the first fluid phase 131. One of skill in the art will appreciate that though the terms “first” and “second” are employed, they are not intended to describe a sequential order or to suggest that only two fluid phases, flow rates etc. may be used, unless otherwise stated. In some embodiments a third fluid phase (or a fourth, a fifth, a sixth, etc.) may be used in conjunction with a first fluid phase and a second fluid phase and may be of any sort described herein. The flow rate of any fluid phase described herein (e.g. first fluid phase 131, the second fluid phase 132) at any given time may be at least about 0 microliters per minute (μL/min) 0.1 μL/min, 0.2 μL/min, 0.3 μL/min, 0.4 μL/min, 0.5 μL/min, 0.6 μL/min, 0.7 μL/min, 0.8 μL/min, 0.9 μL/min, 1 μL/min, 2 μL/min, 3 μL/min, 4 μL/min, 5 μL/min, 6 μL/min, 7 μL/min, 8 μL/min, 9 μL/min, 10 μL/min, 11 μL/min, 12 μL/min, 13 μL/min, 14 μL/min, 15 μL/min, 16 μL/min, 17 μL/min, 18 μL/min, 19 μL/min, 20 μL/min, 21 μL/min, 22 μL/min, 23 μL/min, 24 μL/min, 25 μL/min, 26 μL/min, 27 μL/min, 28 μL/min, 29 μL/min, 30 μL/min, 31 μL/min, 32 μL/min, 33 μL/min, 34 μL/min, 35 μL/min, 36 μL/min, 37 μL/min, 38 μL/min, 39 μL/min, 40 μL/min, 41 μL/min, 42 μL/min, 43 μL/min, 44 μL/min, 45 μL/min, 46 μL/min, 47 μL/min, 48 μL/min, 49 μL/min, 50 μL/min, 60 μL/min, 70 μL/min, 80 μL/min, 90 μL/min, 100 μL/min, 110 μL/min, 120 μL/min, 130 μL/min, 140 μL/min, 150 μL/min, 160 μL/min, 170 μL/min, 180 μL/min, 190 μL/min, 200 μL/min, or the flow rate any fluid phase described herein may take on any value between any two aforementioned values. The flow rates of any fluid phase described herein may be cumulative or subtractive with the flow rates of any other fluid phase described herein.

The droplets 150 of any embodiment may take on any suitable shape. For example, the droplets 150 may be spherical or approximately spherical. The droplets 150 of some embodiments may take on a non-spherical shape, such as an ellipsoid or a disk. The droplets 150 of any embodiment may each have a diameter (the diameter here considered to be the diameter of a perfect mathematical sphere having the same volume as the given droplet) of no more than about 0.1 micrometers (μm), 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 300 μm, 400 μm, 500 μm, 600 μm, 700 μm, 800 μm, 900 μm, 1 millimeters (mm), 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 4 mm, 5 mm, 6 mm, 7 mm, 8 mm, 9 mm, 10 mm, or the droplets 150 may take on a droplet size in between any two of the aforementioned values. Each of the plurality of droplets may have a droplets size from about 0.1 μm to about 200 μm, from about 1 μm to about 150 μm, and or from about 10 μm to about 100 μm.

The droplets 150 of any embodiment may be formed at any suitable rate. In some embodiments, droplets 150 form at a rate of at least about 1 droplet per second (dps), 2 dps, 3 dps, 4 dps, 5 dps, 6 dps, 7 dps, 8 dps, 9 dps, 10 dps, 20 dps, 30 dps, 40 dps, 50 dps, 60 dps, 70 dps, 80 dps, 90 dps, 100 dps, 200 dps, 300 dps, 400 dps, 500 dps, 600 dps, 700 dps, 800 dps, 900 dps, 1,000 dps, 2,000 dps, 3,000 dps, 4,000 dps, 5,000 dps, 6,000 dps, 7,000 dps, 8,000 dps, 9,000 dps, 10,000 dps, 20,000 dps, 30,000 dps, 40,000 dps, 50,000 dps, 60,000 dps, 70,000 dps, 80,000 dps, 90,000 dps, 100,000 dps, 200,000 dps, 300,000 dps, 400,000 dps, 500,000 dps, 600,000 dps, 700,000 dps, 800,000 dps, 900,000 dps, 1,000,000 dps, 2,000,000 dps, 3,000,000 dps, 4,000,000 dps, 5,000,000 dps, 6,000,000 dps, 7,000,000 dps, 8,000,000 dps, 9,000,000 dps, 10,000,000 dps, 20,000,000 dps, 30,000,000 dps, 40,000,000 dps, 50,000,000 dps, 60,000,000 dps, 70,000,000 dps, 80,000,000 dps, 90,000,000 dps, or 100,000,000 dps, or the rate of droplet formation may take on a value between any two of the aforementioned values. In some embodiments, droplets 150 form at a rate of not more than about 100,000,000 droplets per second (dps), 90,000,000 dps, 80,000,000 dps, 70,000,000 dps, 60,000,000 dps, 50,000,000 dps, 40,000,000 dps, 30,000,000 dps, 20,000,000 dps, 10,000,000 dps, 9,000,000 dps, 8,000,000 dps, 7,000,000 dps, 6,000,000 dps, 5,000,000 dps, 4,000,000 dps, 3,000,000 dps, 2,000,000 dps, 1,000,000 dps, 900,000 dps, 800,000 dps, 700,000 dps, 600,000 dps, 500,000 dps, 400,000 dps, 300,000 dps, 200,000 dps, 100,000 dps, 90,000 dps, 80,000 dps, 70,000 dps, 60,000 dps, 50,000 dps, 40,000 dps, 30,000 dps, 20,000 dps, 10,000 dps, 9,000 dps, 8,000 dps, 7,000 dps, 6,000 dps, 5,000 dps, 4,000 dps, 3,000 dps, 2,000 dps, 1,000 dps, 900 dps, 800 dps, 700 dps, 600 dps, 500 dps, 400 dps, 300 dps, 200 dps, 100 dps, 90 dps, 80 dps, 70 dps, 60 dps, 50 dps, 40 dps, 30 dps, 20 dps, 10 dps, 9 dps, 8 dps, 7 dps, 6 dps, 5 dps, 4 dps, 3 dps, 2 dps, 1 dps, or the rate of droplet formation may take on a value between any two of the aforementioned values. The rate of droplet formation may be selected by a user and directed by a controller as described elsewhere herein.

Droplet formation and/or detachment from the membrane may be aided by a shear force perpendicular to the droplet flow direction. For example, in those embodiments in which droplets are formed by a second fluid phase coming into contact with a first fluid phase (such as one residing in a chamber) through a membrane, then a shear force perpendicular to the flow path of the second fluid phase may be used to increase the rate of droplet detachment from the membrane, such as by cross flow movement of the first fluid phase or by agitation of the membrane (such as by vibrating the apparatus or system in which the membrane resides or by moving the membrane individually or some combination thereof).

Droplet formation and/or detachment from the membrane may be further aided by decreasing the interfacial tension of a first fluid phase and a second fluid phase. Interfacial tension between the first fluid phase and the second fluid phase may be increased or decreased by introducing a third fluid phase comprising a surfactant or by incorporating a surfactant into either the first fluid phase or the second fluid phase. A surfactant may be used to decrease the interfacial tension of the first fluid phase and the second fluid phase and thereby increase droplet formation and/or detachment from the membrane. The surfactant may be of any sort described herein including but not limited to anionic surfactants, cationic surfactants, zwitterionic surfactants, and nonionic surfactants. The interfacial tension force may be reduced dynamically as a surfactant adsorbs at the interface between the first and second fluid phases. That is, the interfacial tension force may be governed at least in part by the rate of surfactant adsorption. The total reduction in interfacial tension (and thus its effects on droplet formation and/or detachment from the membrane) is a function of the specific surfactant type and concentration used.

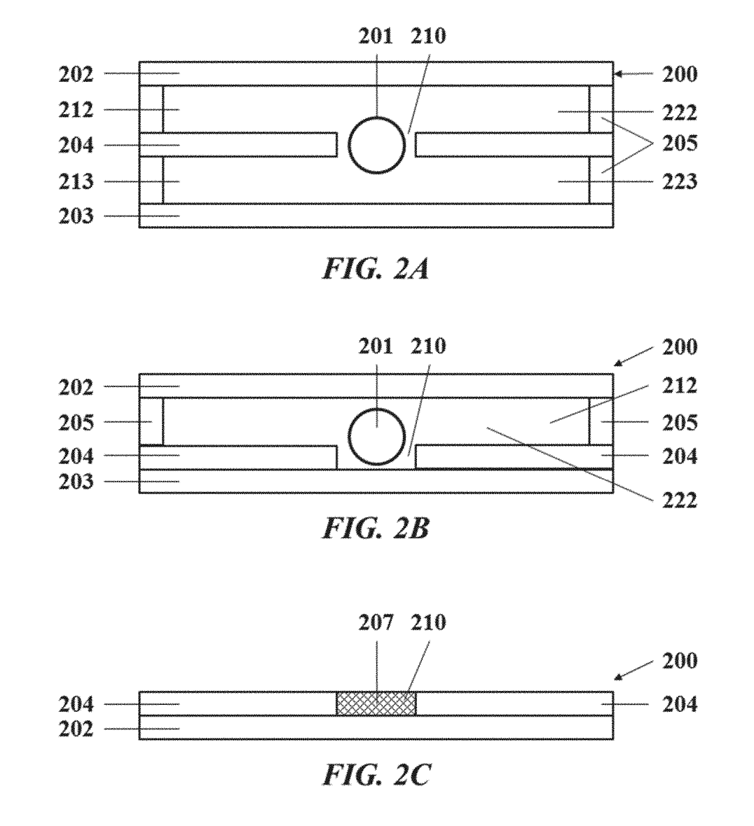

FIGS. 2A-2C show a cross-sectional view of an exemplary support system 200 associated with the methods and systems for biological processing. The support system 200 of this or any embodiment may be a portion of a sample processing unit. The sample processing unit (e.g., via the support system 200) may comprise a plurality of wells (e.g., a plurality of supports) and a fluid flow path in fluid communication with the plurality of wells. Flow of a plurality of droplets through the fluid flow path to the plurality of wells such that the plurality of droplets is deposited within the plurality of wells may be controlled via a controller or may be executed manually. Directing the flow of the plurality of droplets may comprise directing the plurality of droplets along a first channel (such as the first channel 222 seen in FIG. 2A) or a second channel (such as the second channel 223 seen in FIG. 2A) or both and providing a first liquid phase in the first channel ad a second liquid phase in the second channel to retain the plurality of droplets in the plurality of wells. The first liquid phase of may differ from the second liquid phase, though both are preferably immiscible with the droplet and/or the plurality of droplets. At least one heating element may be used to convert electrical energy or electromagnetic energy into thermal energy and thereby subject the plurality of droplets to heating. Such heating may at least in part process the biological sample.

The support system 200 may be used to immobilize a sample or a portion of a sample. As illustrated in FIGS. 2A-2B the sample may comprise one or more droplets 201 of a solution (e.g., an aqueous solution comprising the biological sample or a portion of the biological sample in an emulsion). The one or more droplets 201 may be of any type of droplet described herein including reaction droplets, heating droplets, or empty droplets.

Turning now to FIG. 2A, the support system 200 may comprise a first bounding layer 202 and a second bounding layer 203 between which the droplet 201 may lie in an opening 210 of a support 204. The first bounding layer 202 and the second bounding layer 203 may individually or collectively comprise an optically clear material such as an optically clear plastic (e.g. acrylic, polycarbonate, etc.), a glass, an organic material, etc. In some embodiments, in additional to optical clarity, the material that comprises the first bounding layer 202 or the second bounding layer 203 may be electrically conductive. Furthermore, the first bounding layer 202 and/or the second bounding layer 203 may comprise a thermoelectric material that generates heat upon activation by either being subjected to a potential or being injected with a current. An example of an optically transparent and electrically conductive material that may comprise the first bounding layer 202 or the second bounding layer 203 or both may be indium tin oxide.

The first bounding layer 202 may at least in part demarcate a first channel 222 within which may reside a first fluid 212. Similarly, the second bounding layer 203 may at least in part demarcate a second channel 223 within which may reside a second fluid 213. In some embodiments, support 204 may at least in part demarcate either the first channel 222 or the second channel 223 or bother. The combination of the first bounding layer 202 and the support 204 may at least in part demarcate the first channel 222 and/or the combination of the second bounding layer 203 and the support 204 may at least in part demarcate the second channel 223.

The first bounding layer 202 or the second bounding layer, or both, may individually or collectively comprise a heating element. The heating element(s) may be of any type described herein (e.g., an inductive heating element, a thermoelectric heating element, etc.). The support system 200 may be heated via the first bounding layer 202, the second bounding layer 203, both, or neither. For those embodiments wherein both the first bounding layer 202 and the second bounding layer 203 comprise a heating element, heat may be generated by both layers simultaneously or sequentially or any combination thereof.

Thermal contact between the solution containing the biological sample (in this illustrated embodiment, the droplet 201) and the first bounding layer 202 may be facilitated by a first fluid 212. Similarly, thermal contact between the solution containing the biological sample (e.g., the droplet 201) and the second bounding layer 203 may be facilitated by a second fluid 213. The first fluid 212 and the second fluid 213 may comprise any fluid described herein, such as an oil. The first fluid 212 and the second fluid 213 may comprise different fluids. The fluids comprising the first fluid 212 and the second fluid 213 may differ in their chemical composition, their viscosity, their density, etc. In those cases in which the densities of the first fluid 212 and the second fluid 213 differ, the first fluid 212 may be less dense than the solution containing the biological sample and the second fluid 213 may be more dense than the solution containing the biological sample so that the solution containing the biological sample may rest between the two fluids, for instance, in the opening 210 of the support 204.

The first bounding layer 202 or the second bounding layer 203 or both may comprise a coating (not illustrated) that electrically insulates the first bounding layer 202 or the second bounding layer 203 or both from other elements (e.g., from the coupling element 205, the first fluid 212, the second fluid 213, etc.). For example, the first bounding layer 202 or the second bounding layer 203 or both may comprise a combination of indium tin oxide and polyethylene terephthalate (PET) (e.g., PET-P, PET-G, etc.). The first bounding layer 202 or the second bounding layer 203 or both may comprise carbon, graphite, plastic, metal (e.g., steel, nickel, aluminum, etc.), or any combination thereof. For example, sheets of carbon may be deposited on, coated on, layer onto, sprayed onto, fused to, bound on, or coupled to the first bounding layer 202, the second bounding layer 203, or any component of the support system 200, or any combination thereof. The first bounding layer 202 or the second bounding layer 203 or both may comprise an electrically non-conductive materials, such as one or more plastics, carbon, graphite, etc. In some embodiments, the first bounding layer 202 or the second bounding layer 203 or any component of the support system 200 may be formed via injection molding.

The first bounding layer 202 or the second bounding layer or both may individually or collectively have a thickness of less than or about 1 micrometers (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, or they may take on any value in between. In some embodiments, the first bounding layer 202 or the second bounding layer or both may individually or collectively have a thickness of no more than about 1 micrometers (m), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, or they may take on any value in between. Similarly, the support system 200 in some embodiments has an overall thickness of less than or about 1 micrometers (m), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm. In some embodiments, the support system 200 has an overall thickness of no more than about 1 micrometers (m), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeters (mm), 2 mm, 3 mm, 4 mm, 5 mm, 6 mm, 7 mm, 8 mm, 9 mm, 1 centimeters (cm), 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm.

The first bounding layer 202 and the second bounding layer 203 may each be coupled to the support 204 via a coupling element 205.

The support 204 may comprise a partition of any type described herein. For example, the support 204 may comprise a material mesh (such as nickel, chromium, stainless steel, etc.). The support may be sized and shaped to hold a suitable volume of material (e.g., of the sample, of the solution comprising the sample, etc). In some cases, the support may hold a volume of at least about 0.001 mL, at least about 0.005 mL, at least about 0.01 mL, at least about 0.05 mL, at least about 0.1 mL, at least about 0.5 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL or more. In some cases, the support 204 may hold a volume of at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 0.5 mL, at most about 0.1 mL, at most about 0.05 mL, at most about 0.01 mL, at most about 0.005 mL, at most about 0.001 mL. Moreover, the support 204 may be contained in any suitable volume. In some cases, the support 204 may be circumscribe a volume that is less than or equal to about 50 milliliters (mL), 40 mL, 30 mL, 20 mL, 10 mL, 5 mL, 1 mL, 100 microliters (uL), 10 uL, 1 uL, 500 nanoliters (nL), 100 nL, or 10 nL. The support 204 may circumscribe a volume in the picoliter (pL) or nanoliter (nL) range up to the microliter (uL) range. The volume circumscribed by the support 204 may be at least about 1 pL, 10 pL, 100 pL, 500 pL, 1 nL, 100 nL, 500 nL, 1 uL, 100 uL, 1000 uL, or greater. In some cases, a support 204 may hold a volume that is less than or equal to about 1000 uL, 100 uL, 50 uL, 40 uL, 30 uL, 20 uL, 10 uL, 1 uL, 500 nL, 100 nL, or 1 nL.

The support 204 may be configured to retain a plurality of droplets (droplets that may individually or collectively comprise a biological sample or a portion thereof) before, during, or after heating of the plurality of droplets. At least one heating element (of a possible plurality of heating elements) may be in thermal communication with the plurality of wells.

The coupling element 205 may comprise an adhesive, a glue, a tape, a locking mechanism, a weld, a solder joint, or a stitched region.

The opening 210 may be sized and shaped to receive the droplet 201. In some cases the opening 210 may have a cross-sectional diameter that is slight less than the projected diameter of the droplet 201 such that only a portion of the droplet 201 may fit through the opening 210. The droplet may be immobilized via an interference fit, may be held in place by van der Waals reactions, and/or may be directed and/or supported by capillary forces. As the droplet 201 is immobilized in the support system 200, the droplet may not retain its shape. In those instances in which the droplet 701 does not retain its shape while immobilized in the support system 200, the droplet may take on the shape or a portion of the shape of the opening 210. In some cases, the opening 210 may only allow a droplet 201 to enter from one side of the opening 210 (e.g., a first side) while precluding it from exiting from another side of the opening 210 (e.g., a second side). In some cases, the opening 210 may permit unidirectional flow.

The opening 210 may permit fluid communication between the first channel 222 and the second channel 223 such that a first opening in the first channel 222 is in fluid communication with a second opening in the second channel 223.

FIG. 2B illustrates a support system 200 similar to that shown in FIG. 2A. The support system 200 comprises a first bounding layer 202 coupled to a support 204 via a coupling element 205, a second bounding layer 203 onto which the support 204 is coupled, an opening 210, and a first fluid 212. Each of the elements of the support system 200 of FIG. 2B (e.g., the first bounding layer 202, the second bounding layer 203, the support 204, the coupling element 205, the opening 210, the first fluid 212, the first channel 222, etc.) may be of any type described herein.

FIG. 2C illustrates a support system 200 comprising a first bounding layer 202, a support 204 bound to the first bounding layer 202, an opening 210 with a directing element 207 that directs one or more droplets and/or a portion of a solution containing a biological sample into the support 204.

The directing element 207 may comprise a hygroscopic material such as any described herein (e.g., sugar, etc.). The direct element 207 may comprise one or more fibers (such as cellulose fibers) to cause a droplet and/or a portion of the solution containing the biological sample to be directed to the support 204. The direct element 207 may comprise a structure comprising surface features (e.g., stair steps) that cause a droplet and/or a portion of the solution containing the biological sample to be directed to the support 204.

Any of the support systems 200 described herein may be used for one or more methods of facilitating a chemical or biological reaction on a biological sample.

In some embodiments, the support system 200 may be part of a sample processing unit comprising a fluid flow path in fluid communication with the support system 200, wherein the support 200 comprises a plurality of wells, and wherein an individual well of the plurality of wells comprises a hygroscopic material that directs a given droplet of a plurality of droplets to said individual well.

In some embodiments, an apparatus for facilitating a chemical or biological reaction on a biological sample comprises a support system 200 that comprises a plurality of wells, wherein an individual well of the plurality of wells comprises a hygroscopic material that (i) directs a given droplet of a plurality of droplets to the individual well, and (ii) retains said given droplet in the individual well during said chemical or biological reaction.

In some embodiments, a sample processing unit comprises a first fluid flow path and a second fluid flow path in fluid communication with a support system 200, wherein the support system 200 comprises a plurality of wells, and wherein an individual well of the plurality of wells comprises a first opening adjacent to the first fluid flow path and a second opening adjacent to the second fluid flow path. Such embodiments may further comprise a first and second fluid flow paths along which the plurality of droplets may flow (either along the first fluid flow path or the second fluid flow path or both). A given droplet from the plurality of droplets may be directed from the fluid flow path it is flowing along (e.g., the first fluid flow path, the second fluid flow path, or both) into the individual well from the plurality of wells through either the first opening or the second opening, depending on the path taken by the given droplet. Furthermore, a first fluid phase may be disposed within the first fluid path and a second fluid phase may be disposed within the second fluid path, thereby retaining the given droplet in the individual well.

In some cases, a subset of the plurality of wells comprises components necessary for conducting a chemical or biological reaction on the biological sample. For example, the biological sample may comprise a nucleic acid molecule and the at least a subset of the plurality of partitions may comprise the biological sample and components necessary for a nucleic acid amplification reaction, with examples of nucleic acid amplification reactions and necessary components for nucleic acid amplification provided elsewhere herein. Where the at least a subset of the plurality of partitions comprises components necessary for conducting a chemical or biological reaction on the biological sample, the method may further comprise conducting the chemical or biological reaction on the biological sample (e.g., conducting nucleic acid amplification reaction(s) in the at least a subset of the plurality of partitions, with or without the aid of thermal cycling). Moreover, the method may also comprise detecting one or more signals indicative of the chemical or biological reaction. Any suitable detector and detection modality can be used, including examples of such provided elsewhere herein.

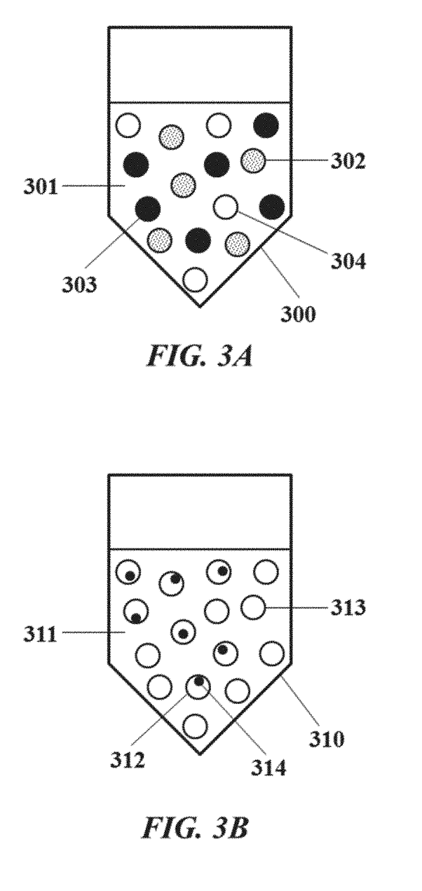

Examples of two populations of droplets are schematically shown in FIG. 3A and FIG. 3B. As shown in FIG. 3A, a vessel 300 comprises a continuous phase 301 comprising a population of droplets. The population of droplets comprises three types of droplets: reaction droplets 302, monitoring droplets 303 and empty droplets 304 (e.g., droplets not containing a portion of the biological sample).

The reaction droplets 302 may comprise a portion of a biological sample and components necessary for conducting a chemical or biological reaction on the biological sample. As an alternative, the reaction droplets 302 include the entirety of the biological sample.

The monitoring droplets 303 may comprise a detectable moiety. The detectable moiety of the monitoring droplets 303 may comprise a detectable moiety capable of detecting temperature, temperature differences, heat, heat flux, or a thermal dose, or any combination thereof. For example, monitoring droplets may comprise a type of thermal liquid crystal (e.g., a nanoparticle of a thermal liquid crystal) that reflects a light having a first wavelength at a first temperature and may reflect light having a second wavelength at a second temperature, thereby monitoring temperature of the monitoring droplet. In some embodiments, a signal from the monitoring droplets may be indicative of a state of the population of droplets. For example, the signal from one or more monitoring droplets may indicate the temperature of the solution, the temperature of the vessel, the temperature of at least a subset of the population of droplets (e.g., nearest neighboring droplets, droplets of a first type, droplets of a second type, droplets of a third type, etc.), or any combination thereof.

As shown in FIG. 3B, a vessel 310 comprises a continuous phase 311 comprising a population of droplets. The population of droplets comprises two types of droplets: reaction droplets 312 and empty droplets 313. The reaction droplets 312 comprise a portion of a biological sample, components necessary for conducting a chemical or biological reaction on the biological sample, and one or more detectable moieties 314 as described herein. As an alternative, the reaction droplets 312 include the entirety of the biological sample.

In various aspects, a solution can have any suitable volume. In some cases, the volume of a solution may be kept relatively low in order to, for example, accommodate small sample sizes and/or permit faster processing times. For example, the volume of a solution may be at most about 100 mL, at most about 50 mL, at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 0.7 mL, at most about 0.5 mL, at most about 0.3 mL, at most about 0.1 mL, at most about 0.05 mL, at most about 0.01 mL, at most about 0.005 mL, at most about 0.001 mL or less. In some cases, the volume of a solution may be maximized in order to, for example, accommodate large sample sizes without separate processing. For example, the volume of a solution may be at least about 0.001 mL, at least about 0.005 mL, at least about 0.01 mL, at least about 0.05 mL, at least about 0.1 mL, at least about 0.3 mL, at least about 0.5 mL, at least about 0.7 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL, at least about 50 mL, at least about 100 mL or more.

In some embodiments, methods for facilitating a chemical or biological reaction further comprise bringing an aqueous phase in contact with a continuous phase to generate an emulsion comprising aqueous droplets dispersed in the continuous phase. In some cases, the aqueous and continuous phases can be brought into contact at an intersection or junction of a first channel, second channel and third channel whereby the first channel provides the aqueous phase to the junction and the second channel provides the continuous phase to the junction. Due to the immiscibility of the aqueous phase in the continuous phase, aqueous droplets are generated in the continuous phase at the junction and can flow from the junction through the third channel. In some cases, the aqueous and continuous phases can be brought into contact by alternately opening and closing a port or channel that provides discontinuous aliquots of the aqueous phase to a bulk continuous phase.

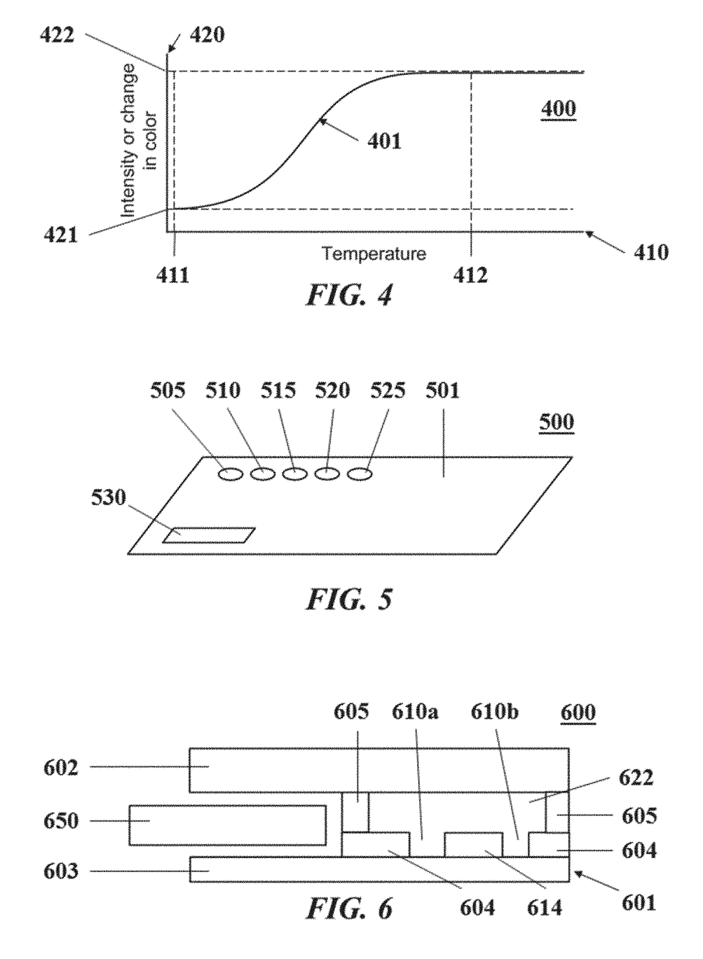

FIG. 4 shows a graph 400 demonstrating an exemplary embodiment of the signal transmitted by a detectable moiety of any sort described herein as a function of temperature. The graph 400 comprises two axes, a first axis 410 representing temperature indicated by the temperature indicator (e.g., the temperature of the detectable moiety, the temperature of the system, the temperature of the substrate to which the detectable moiety is coupled, the temperature of one or more droplets, etc.), and a second axis 420 representing signal intensity. In the illustrated embodiment, the function 401 represents the relationship between the detectable moiety's generated signal, in this case a light intensity or color from an optically detectable detectable moiety and temperature; however, one of skill in the art will appreciate that of the signal generated by the detectable moiety may arise from any detectable moiety as described herein. As a non-limiting, the detectable moiety of the illustrated embodiment may comprise temperature indicator such as a thermal liquid crystal. The function 401 of the temperature indicator may take on any shape, such as the sigmoidal curve shown in the illustration. The function 401 may have lower 411 and upper 412 operative bounds with respect to temperature such that the signal indicated by the temperature indicator at or below a first temperature 411 has a first response 421 and the signal indicated by the temperature at or above a second temperature 412 has a second response 422. Between the first temperature 411 and the second temperature 412 lies the operative range of the temperature indicator and thus the operative range of responses may lie between the first response 421 of the temperature indicator and the second response 422 of the temperature indicator. The operative temperature range may be of any described herein.

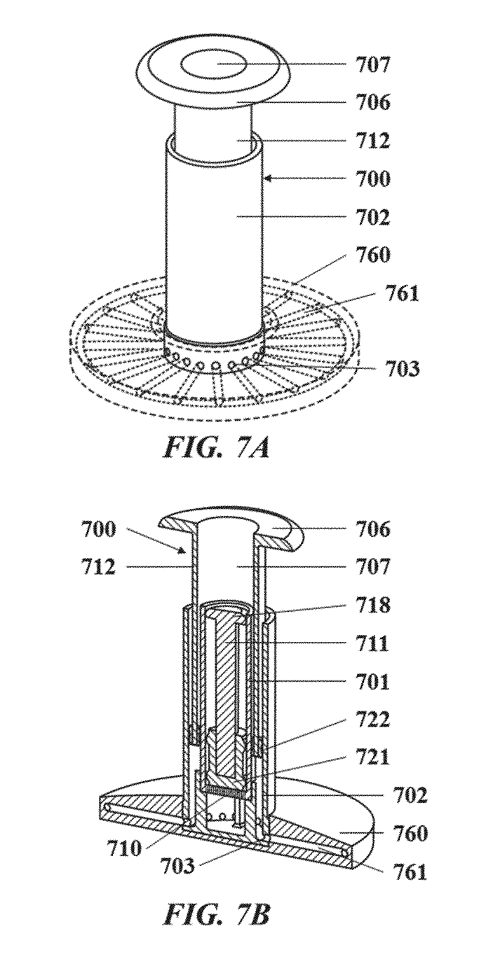

FIG. 5 shows a temperature monitoring system 500 comprising a plurality of temperature indicators 505, 510, 515, 520, 525, 530 (each comprising a detectable moiety selected from any described herein) coupled to a substrate 501. The substrate 501 may comprise a vessel as described herein, a support as described herein, or the substrate 501 may comprise any surface of any system described herein (e.g., disposed along a vessel surface, a laminate layer a heating layer, etc.). The temperature indictors 505, 510, 515, 520, 525, 530 may individually or collectively comprise one or more resistors, one or more thermocouples, one or more thermistors, one or more diodes, one or more transistors, one or more infrared emitters, one or more detectable moieties (e.g., the temperature indicators 505, 510, 515, 520, 525, 530 may comprise a fluorescent dye or a fluorescent detector), one or more liquid crystals (e.g., one or more thermochromic liquid crystal particles), or one or more temperature sensitive coatings (e.g., a paint, a membrane, a thin film, a layer, etc.). The temperature indicators 505, 510, 515, 520, 525, 530 may individually or collective transmit one or more temperature sensitive parameters. Temperature sensitive parameters may include but are not limited to an electrical resistance, an electrical potential, an electrical current, an open circuit voltage, a color, a light intensity, or any combination thereof. For instance, the temperature indicators 505, 510, 515, 520, 525, 530 may comprise a thermal liquid crystal that reflects light of a first color and/or intensity at a first temperature and light of a second color and/or intensity at a second temperature. The temperature indicators 505, 510, 515, 520, 525, 530 may take on any shape such as a circle (as temperature indicators 505, 510, 515, 520, 525 as illustrated take on), an oval an ellipse, a square, a rectangle (as temperature indicator 530 as illustrated shows), a triangle, a line, a particle, two or more particles, or a point (as would be in the case in those embodiments utilizing thermocouples, thermistors, etc.), or any combination thereof.

One or more temperature indicators 505, 510, 515, 520, 525, 530 may be used such that at least a first temperature indicator (e.g., 505) has a first temperature range (e.g., from about 30° C. to about 50° C.) and a second temperature indicator (e.g., 510) has a second temperature range (e.g., from about 50° C. to about 70° C.). In some embodiments, the first temperature range and the second temperature range have no operative overlap. For example, the first temperature indicator 505 may operatively indicate temperature from 30° C. to less than 50° C. and the second temperature indicator 510 may operatively indicate temperature from 50° C. to less than 70° C. In some embodiments, the first temperature range and the second temperature range have some operative overlap. For example, the first temperature indicator 505 may operatively indicate temperature from 30° C. to 60° C. and the second temperature indicator 510 may operatively indicate temperature from 50° C. to 70° C., such that a portion of the first temperature range and the second temperature of the range are the same. In those embodiments wherein two or more temperature indicators operatively indicate temperature from overlapping temperature ranges, the detector (not illustrated) detecting the temperature may use the results of a first temperature range to calibrate the results of a second temperature range. In those embodiments wherein two or more temperature indicators operatively indicate temperature from overlapping temperature ranges, the detector (not illustrated) detecting the temperature may average the results indicated by the first temperature indicator and the results indicated by the second temperature indicator.

The temperature indicators may individually or collectively have an operative range from about 0° C. to about 10° C., from about 10° C. to about 20° C., from about 20° C. to about 30° C., from about 30° C. to about 40° C., from about 40° C. to about 50° C., from about 50° C. to about 60° C., from about 60° C. to about 70° C., from about 70° C. to about 80° C., from about 80° C. to about 90° C., from about 90° C. to about 100° C., from about 100° C. to about 110° C., from about 110° C. to about 120° C., from about 120° C. to about 130° C., from about 130° C. to about 140° C., from about 140° C. to about 150° C., from about 150° C. to about 160° C., from about 160° C. to about 170° C., from about 170° C. to about 180° C., from about 180° C. to about 190° C., from about 190° C. to about 200° C., from about 200° C. to about 210° C., or the temperature indicators may individually or collectively have an operative range between any two aforementioned values. In some embodiments the temperature indicators may individually or collectively have an operative range from about 0° C. to about 5° C., from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., from about 70° C. to about 75° C., from about 75° C. to about 80° C., from about 80° C. to about 85° C., from about 85° C. to about 90° C., from about 90° C. to about 95° C., from about 95° C. to about 100° C., from about 100° C. to about 105° C., from about 105° C. to about 110° C., from about 110° C. to about 115° C., from about 115° C. to about 120° C., from about 120° C. to about 125° C., from about 125° C. to about 130° C., from about 130° C. to about 135° C., from about 135° C. to about 140° C., from about 140° C. to about 145° C., from about 145° C. to about 150° C., from about 150° C. to about 155° C., from about 155° C. to about 160° C., from about 160° C. to about 165° C., from about 165° C. to about 170° C., from about 170° C. to about 175° C., from about 175° C. to about 180° C., from about 180° C. to about 185° C., from about 185° C. to about 190° C., from about 190° C. to about 195° C., from about 195° C. to about 200° C., from about 200° C. to about 205° C.

The temperature indicators 505, 510, 515, 520, 525, 530 may be monitored via any detector described herein. For example, in some embodiments wherein one or more temperature indicators 505, 510, 515, 520, 525, 530 individually or collectively comprises one or more thermal liquid crystal, the temperature indicators 505, 510, 515, 520, 525, 530 may be monitored by a camera.

Though illustrated as coupled to a substrate 501, one or more detectable moieties may be disposed within the sample, the vessel, one or more wells from the plurality of wells, or one or more droplets of any embodiment. For example, one or more monitoring droplets may individually or collectively comprise one or more of the detectable moieties described herein.

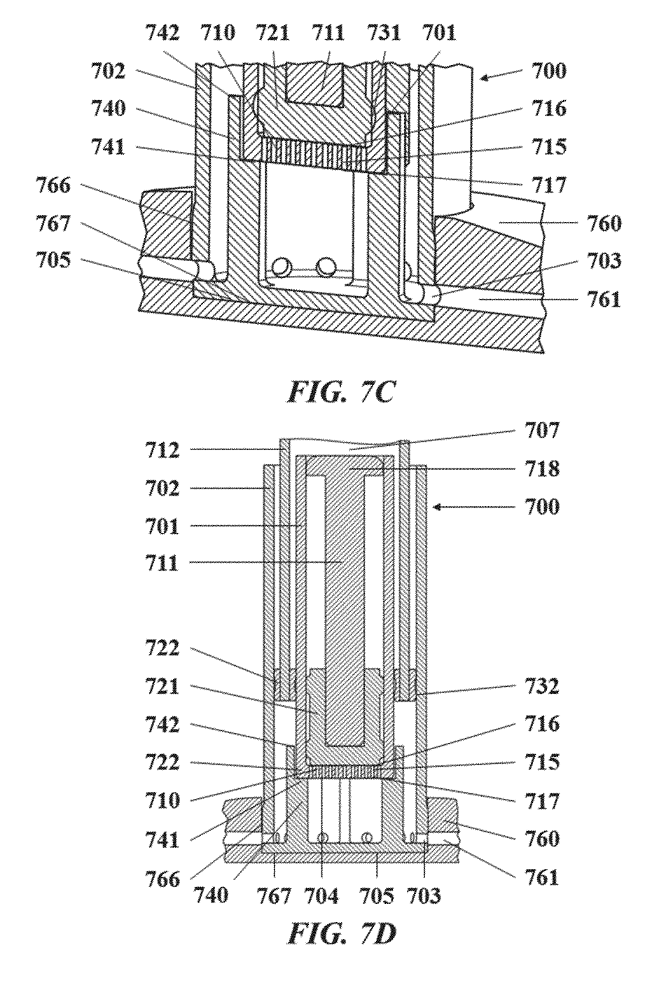

FIG. 6 shows a cross-sectional view of an exemplary embodiment of a temperature monitor 600 comprising a support system 601 (similar to those illustrated in FIGS. 2A-2C). The support system may be of any sort described herein. In the illustrated embodiment of FIG. 6 the support system comprises a first bounding layer 602 and a second bounding layer 603 coupled via a coupling element 605 to a support 604 comprising two wells 610a and 610b separated by an intermediate support 614. A first channel 622 may demarcated by at least the bounds of the first bounding layer 602 and the second bounding layer 603. The temperature of the temperature monitor may be indicated by the temperature indicator 650 coupled to the temperature monitor. The temperature indicator may be of any type described herein, such as, for example, a thermocouple or a thermistor (e.g., a negative thermal coefficient thermistor, a positive thermal coefficient thermistor, etc.).

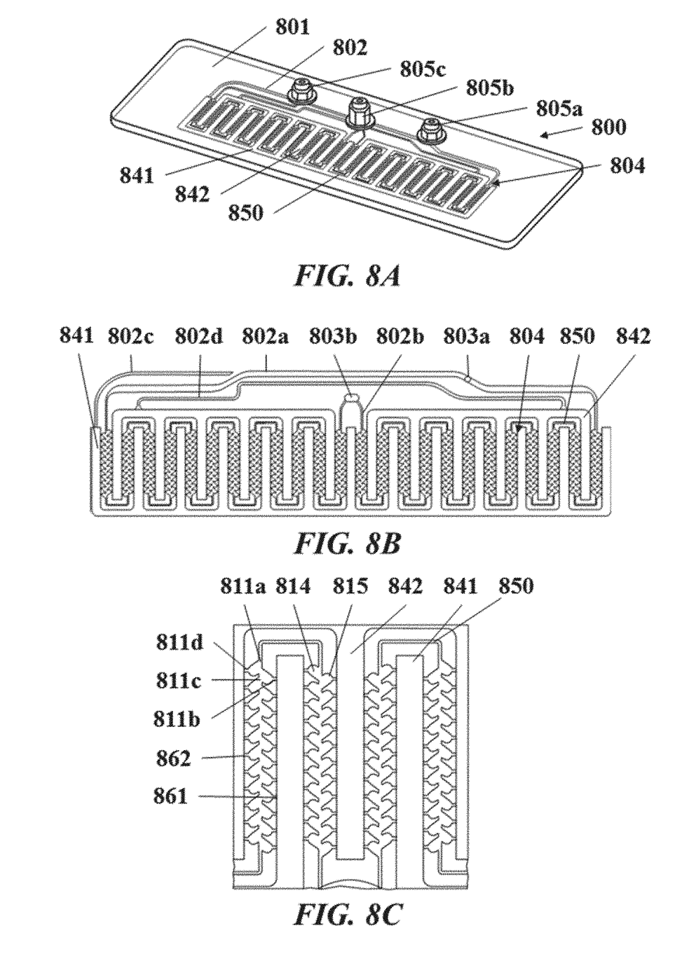

FIG. 7A shows a perspective view of an exemplary droplet generating apparatus 700. The droplet generating apparatus 700 comprises a first chamber with a first actuator (best seen in FIGS. 7B and 7D) and a second chamber 702 with a second actuator 712.

As illustrated, the second actuator 712 has an actuator head 706 that comprises a flanged region disposed at the top of the actuator 712. (As used herein the use of the terms “top,” “bottom,” and “side” are made with respect to the illustration and are not intended to suggest a singular orientation for the apparatus 700 as a whole. Reference to the “top,” “bottom,” and/or “side” in the descriptions herein should be made with respect to the illustrated embodiment.) The actuator head 706 may be configured to easily fit within the hand of an adult human being. In some embodiments, the actuator head 706 may be coupled to a mechanical actuator (such as a syringe pump) to aid in the actuation of the first actuator or the second actuator 712 or both.

The second actuator 712 may have a channel 707 that extends from the top of the actuator 712 along a characteristic axis of the actuator 712 (e.g., a central axis, a side axis) through at least a portion of the actuator 712. In some embodiments, the channel 707 extends all the way through the second actuator 712. The channel 707 may be sized and/or shaped to receive, house, hold, and/or couple to the first chamber 701 or the first actuator 711 or both.

The second actuator 712 may be slidably coupled to the second chamber 702 in which it is disposed. Actuation of the second actuator 712 may direct a fluid phase (e.g., a fluid phase comprising a plurality of droplets) disposed within the second chamber 702 to exit the chamber 702 through one or more openings 703. Upon exiting the chamber 702, the fluid phase may enter the support 760 (also referred to herein as a “disk,” a “cartridge,” a “sample holder,” and a “storage unit”). The fluid phase may enter the support 760 by way of one or more openings 703 aligned with one or more channels 761 within the support 760.

The support 760 may be of any type described herein. Though illustrated as circular, the support may have any shape including but not limited to those comprising a circle, an oval, an ellipse, a triangle, a square, a rectangle, a pentagon, a hexagon, an octagon, a polygon, or any combination thereof. The support 760 may be coupled to the first chamber or the second chamber 702 (as illustrated). Coupling to or of the support 760 to another structure (e.g., the second chamber 702) may be temporary or permanent. The support 760 may be releasably coupled to the first chamber or the second chamber 702. In some embodiments, the support 760 rotates about the first chamber or the second chamber 702 or both. The first chamber or the second chamber 702 or both may, in some embodiments, rotate about the support 760. The support 760 may have a top and/or bottom surface that is optically clear such that a detector (not illustrated) in optical communication with the support 760 may detect an optical signal generated from within the support 760 (for example, by a detectable moiety within a droplet).

FIG. 7B shows a cut perspective view of the exemplary droplet generating apparatus 700 of FIG. 7A. From this perspective, one can see that the droplet generating apparatus 700 comprises a first chamber 701 with a first actuator 711 disposed therein and a second chamber 702 with a second actuator 712 disposed therein. The first chamber 701 is further disposed within a channel 707 of the second actuator 712 such that the first chamber 701 and its actuator 711 reside within the second actuator 712.

The first actuator 711 may comprise an actuator head 718. The actuator head 718 may be flanged. The actuator head 718 may be configured to easily fit within the hand of an adult human being. In some embodiments, the actuator head 718 may be coupled to a mechanical actuator (such as a syringe pump) to aid in the actuation of the first actuator 711 or the second actuator 712 or both.

The first actuator 711 may be slidably coupled to the first chamber 701. Actuation of the first actuator 711 may be along a characteristic axis of the first chamber 701 (e.g., along a central axis of the first chamber 701, along an axis defined by a side of the first chamber 701, etc.). Actuation of the first actuator 711 may be spatially and/or temporally linear or non-linear or any combination thereof. Actuation of the first actuator 711, in some embodiments, may be at a constant rate, while in other embodiments, the first actuator 711 may be actuated with a first acceleration, brought to a constant velocity, then brought to a stop with a second acceleration. Other comparable actuation regimes will be appreciated by one of skill in the art.

Actuation of the first actuator 711 or the second actuator 712 or both may be initiated by a user or a machine. In some embodiments actuation of the first actuator 711 or the second actuator 712 or both may be controlled by a controller, such as any controller described herein. Actuation of the first actuator 711 may proceed actuation of the second actuator 712 in some embodiments. In some embodiments, actuation of the first actuator 711 follows the actuation of the second actuator 712. In some embodiments, actuation of the first actuator 711 occurs simultaneously as actuation of the second actuator 712. Actuation of the first actuator 711 may proceed, follow, or occur simultaneously as actuation of the second actuator 712 in any combination. For example, in the embodiments wherein a first fluid phase (e.g., a continuous phase fluid) is already disposed within the second chamber 702, the first chamber 701 may contain a second fluid phase (e.g., an aqueous phase fluid, a fluid comprising the biological sample or a portion thereof) that is pushed through the membrane 710 disposed at the bottom of the first chamber 701 via actuation of the first actuator 711 so that the second fluid phase comes into contact with the first fluid phase within the second chamber 702, thereby creating one or more droplets. The one or more droplets residing within the second chamber 702 may be directed out to the support 760 via one or more openings 703 in the second chamber 702. (Though the illustrated embodiment shows the one or more openings 703 of the second chamber 712 along a side surface toward the bottom of the second chamber 702, the one or more openings 703 may be disposed at any point on the second chamber 702, such as the bottom surface of the second chamber 702.) The support 760 may comprise one or more channels 761 that operatively align with the one or more openings 703.

Coupled to the bottom of the first actuator 701 may be a first actuator tip 721. Similarly, the second actuator 702 may comprise a second actuator tip 722. The first 721 and/or second actuator tip 722 may interface with one or more fluids (e.g., a first fluid phase, a second fluid phase, etc.). As such the first 721 and/or second actuator tip 722 may comprise a biologically inert and/or a corrosion resistant material (e.g., plastic, rubber, etc.).

FIG. 7C shows a close-up view of the bottom of the exemplary droplet generating apparatus 700 of FIG. 7A. The droplet generating apparatus 700 may comprise a first chamber 701 with a first actuator 711 and a second chamber 702 with a second actuator (seen in FIGS. 7A, 7B, and 7D).

The first chamber 701 may terminate in a membrane 710 disposed along at least a portion of its bottom surface. The membrane 710 may be of any type described herein. The membrane 710 may comprise at least one opening 715 with a first side 716 oriented toward the interior of the first chamber 701 and a second side 717 oriented toward the exterior of the first chamber 701, which in some embodiments (such as the illustrated embodiment) is equivalent to the interior of the second chamber 702.

The first chamber 701 may physically engage the first actuator 711. Said physical engagement may be via the actuator tip 721 in a sealing engagement with the chamber 701. Such sealing engagement may be facilitated by a protrusion 731 (also referred to herein as a “seal”) on the actuator tip 721. The seal 731 may cause an interference fit to occur between the actuator tip 721 and the chamber 701. The seal 731 and the actuator tip 721 may comprise an integral component. In some embodiments, the seal 731 is coupled to the actuator tip 721 (via stitching, welding, soldering, press fitting, overmolding, adhesion, etc.). The seal and/or actuator tip 721 may be made of a biologically and/or chemically inert and/or corrosion resistant material such a plastic or a rubber.

The first chamber 701 may couple to or rest upon an actuation stop 740 within the second chamber 702. More specifically, the first chamber 701 may couple to or rest upon a first surface 741 of the actuation stop 740. Similarly, the second actuator 712 may couple to or rest upon a second surface 742 of the actuation stop 740. The actuation stop 740 may limit the actuation of the actuator 712 in one direction (e.g., a direction in which actuation may generate droplets). Coupling of the first chamber 701 and/or the second actuator 712 to the actuation stop 740 may provide for a sealing engagement so that no fluid may pass through the region of coupling.

The actuation stop 740 may comprise a plurality of actuation stop elements. Each of the plurality of actuation stop elements may comprise an elongate structure with a bottom coupled to the chamber 702 and a top comprising the first surface 741 and the second surface 742 wherein one or more actuating elements may rest on or couple to. The first surface 741 and the second surface may be at differing heights (such as is the case in the present illustrated embodiment). In some embodiments, the second surface 742 extends past the first surface 741. In some embodiments, the first surface 741 extends past the second surface 742. In some embodiments, the first surface 741 and the second surface 742 are at approximately the same height.

The second chamber 702 may couple to a support 760 via either a side coupling 766 or a bottom coupling 767 (via, for instance, the bottom most surface 705 of the chamber 702) or both. Coupling of the support 760 may be via mechanical coupling or through chemical coupling. In some embodiments, coupling of the support 760 to the chamber 702 is through an interference fit. In some embodiments, coupling of the support 760 to the chamber 702 is at least partially facilitated by an adhesive. Some embodiments may employ means by which to align the support 760 to the chamber 702 including one or more features (e.g., slots, channels, protrusions, divots, pins, holes, etc.) disposed on the chamber 702 and a set of one or more matching features disposed on the support 760 (e.g., a protrusion matched to a slot, a hole matched to a pin, etc.). In some embodiments, the side coupling 766 may comprise a screw-like thread such that the chamber 702 and the support may be threadably engaged and operatively coupled. In some embodiments, the alignment of one or more holes 703 of the chamber 702 with one or more channels 761 of the support 760 may facilitate coupling.

FIG. 7D shows a cut side view of the exemplary droplet generating apparatus 700 of FIG. 7A. The droplet generating apparatus 700 comprises a first chamber 701 (with a first actuator 711) disposed within a channel 707 of a second actuator 712 itself disposed within a second chamber 702. The first actuator 711 may terminate in an actuator tip 721. The first actuator tip 721 may be sized and/or shaped to match the size and/or shape of an interior surface of the first chamber 701 in which it is disposed. The second actuator 712 may terminate in a second actuator tip 722 that may be sized and/or shaped to match the size and/or shape of an interior surface of the second chamber 702 in which it is disposed. In some embodiments the second actuator tip 722 is further sized and/or shaped to match the size and/or shape of an exterior surface of the first chamber 721. For example, if the first chamber 701 has an approximately cylindrical exterior surface and the second chamber 702 has an approximately cylindrical interior surface, then a second actuator tip 722 may take on a ring-like cross-section where the outer circle of the ring-like cross-section approximately matches the circle defined by the cross-section of the second chamber 702 and the inner circle of the ring-like cross-section approximately matches the circle defined by the cross-section of the first chamber 701.

The first actuator tip 721 and the second actuator tip 722 may be of any type described herein. For example, the second actuator tip 722 may comprise a protrusion 732 that provides for a sealing engagement with the chamber 702. The protrusion 732 may cause an interference fit to occur between the actuator tip 722 and the chamber 702. The seal 732 and the actuator tip 722 may comprise an integral component, though in some embodiments the seal 732 and the actuator tip 722 comprise distinct parts. In some embodiments, the protrusion 732 is coupled to the actuator tip 722 (via stitching, welding, soldering, press fitting, overmolding, adhesion, etc.). The protrusion 732 and/or actuator tip 722 may be made of a biologically and/or chemically inert and/or corrosion resistant material such a plastic or a rubber.

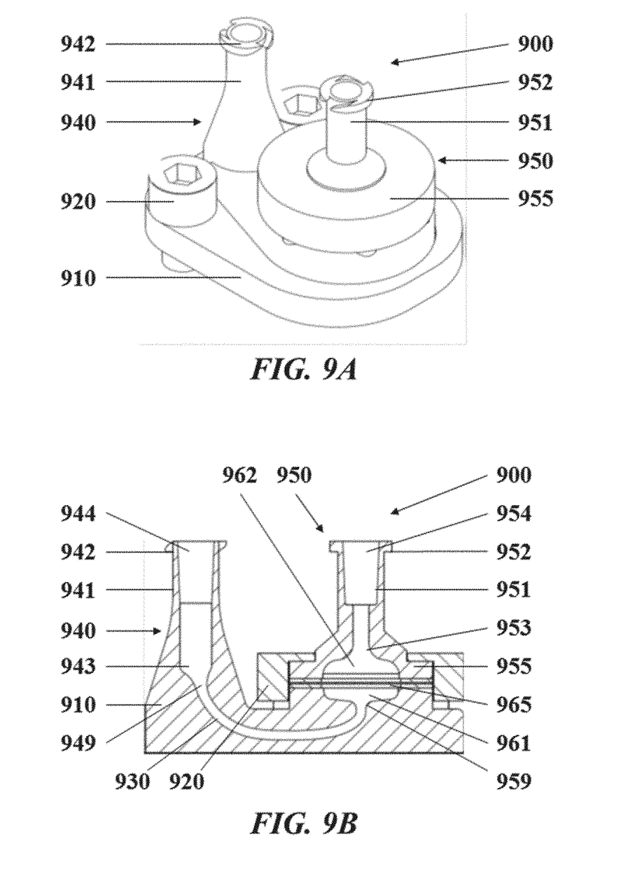

FIG. 8A shows a perspective view of an exemplary embodiment of a support system 800 comprising a plurality of wells 804. The support system 800 may further comprise a substrate 801 and one or more coupling elements (in this case, there are three such coupling elements 805a, 805b, 80c), the one or more coupling elements 805a, 805b, 805c enabling the fluidic coupling of the support system 800 to one or more other elements (e.g., a droplet generating apparatus, one or more tubes, a vacuum, a pump, etc.). The substrate 801 may further comprise one or more channels 802 leading from the coupling elements 805a, 805b, 805c to the plurality of wells.

The support system 800 may further comprise a first fluid flow path 841 and a second fluid flow path 842. The first fluid flow path 841 may couple (e.g., fluidically couple, operatively couple, etc.) to the plurality of wells 804 on a first side of the plurality of wells 804. The second fluid flow path 842 may couple (e.g., fluidically couple, operatively couple, etc.) to the plurality of wells 804 on a second side of the plurality of wells 804. The interface between the first fluid flow path 841 and the plurality of wells 804 may comprise a first semipermeable membrane 861 (best seen in FIG. 8C). The interface between the second fluid flow path 842 and the plurality of wells 804 may comprise a second semipermeable membrane 862 (best seen in FIG. 8C). The first semipermeable membrane 861 or the second semipermeable membrane 862, or both, may comprise a membrane that is permeable to a first fluid phase (e.g., air, oil, an aqueous solution, etc.) and impermeable to a second fluid phase (e.g., air, oil, an aqueous solution, etc.). For example, the first semipermeable membrane 861 or the second semipermeable membrane 862, or both, may allow air through but not allow a liquid through such that as a first fluid phase, such as an oil, is flowed along a fluid flow path, to the plurality of wells, then finally to the semipermeable membrane (via, for example, a channel disposed between an individual well and the semipermeable membrane), the remaining air in the fluid flow path is pushed through the semipermeable membrane but the first fluid phase, in this case an oil, does not. This may fill in the well and allow the first fluid phase to remain in place. The first fluid phase may comprise one or more droplets as described herein.

The semipermeable membrane may comprise at hydrophobic membrane. At least a portion of the semipermeable membrane may comprise a hydrophobic surface. For example, the semipermeable membrane may comprise a first surface and a second surface wherein the first surface or the second surface or both have at least a portion that is hydrophobic.

The semipermeable membrane may comprise a hydrophilic membrane. At least a portion of the semipermeable membrane may comprise a hydrophilic surface. For example, the semipermeable membrane may comprise a first surface and a second surface wherein the first surface or the second surface or both have at least a portion that is hydrophilic.

In some embodiments, a first fluid phase (e.g., air, oil, an aqueous solution, etc.) is disposed within the first fluid flow path 841 and a second fluid phase (e.g., air, oil, an aqueous solution, etc.) is disposed within the second fluid flow path 842.

The first fluid phase may have a first fluidic property (e.g., density, viscosity (kinematic, dynamics, etc.), temperature, pressure, specific volume, specific weight, specific gravity, etc.) and the second fluid phase may have a second fluidic property (e.g., density, viscosity (kinematic, dynamics, etc.), temperature, pressure, specific volume, specific weight, specific gravity, etc.) that differs from the first fluidic property of the first fluid phase. For example, the first fluid phase may comprise a fluid with a first density greater than the density of the individual droplet from the plurality of droplets and the second fluid phase may comprise a fluid with a second density less than the density of the individual droplet from the plurality of droplets. As such, the individual droplet from the plurality of droplets may be retained within an individual well of the plurality of wells 804. One of skill in the art will appreciate that other such combinations of first fluidic properties and second fluidic properties may be used to retain the individual droplet within the individual well such as a first pressure and a second pressure, a first flow rate and a second flow rate, etc.

In some embodiments, the support system 800 may further comprise a well fluid flow path 850 that is in fluid communication with the plurality of wells 804. In some embodiments, one or more droplets may be directed along the well fluid flow path 850. In some embodiments, one or more droplets may be directed along the first fluid flow path 841. In some embodiments, one or more droplets may be directed along the second fluid flow path 842.

The support system 800 may comprise any support described herein. The support system 800 may comprise any number of wells 804. For example, the support system 800 may comprise at least 1 well, 2 wells, 3 wells, 4 wells, 5 wells, 6 wells, 7 wells, 8 wells, 9 wells, 10 wells, 20 wells, 30 wells, 40 wells, 50 wells, 60 wells, 70 wells, 80 wells, 90 wells, 100 wells, 200 wells, 300 wells, 400 wells, 500 wells, 600 wells, 700 wells, 800 wells, 900 wells, 1,000 wells, 2,000 wells, 3,000 wells, 4,000 wells, 5,000 wells, 6,000 wells, 7,000 wells, 8,000 wells, 9,000 wells, 10,000 wells, 20,000 wells, 30,000 wells, 40,000 wells, 50,000 wells, 60,000 wells, 70,000 wells, 80,000 wells, 90,000 wells, 100,000 wells, or the number of wells 804 may take on a value between any two aforementioned values. An individual well from the plurality of wells 804 may be individually addressable (e.g., individually addressable by a fluid handling device, such that the fluid handling device can correctly identify a well and dispense appropriate fluid materials into the well). Furthermore, the contents of an individual well from the plurality of wells 804 may be detectable (e.g., comprise a detectable moiety, be in sensing communication with a detector, etc.).

FIG. 8B shows a top view of the flow paths of the exemplary embodiment of the support system 800 comprising a plurality of wells 804 shown in FIG. 8A. The illustrated embodiment emphasizes how one or more fluid phases may flow around the support system 800.

The support system 800 may comprise one or more openings such as openings 803a and 803b through which a solution (e.g., a solution comprise a plurality of droplets) may be flowed into or out of Between a first opening 803a and a second opening 803b may be a channel through which a solution may be flowed that is in fluid communication with the plurality of wells 804. The first opening 803a may be fluidically coupled to the plurality of wells 804 via a first channel 802a and the second opening 803b may be fluidically coupled to the plurality of wells 804 via a second channel 802b.

Connecting the plurality of wells 804 to one another may be a well fluid flow path 850 through which one or more droplets may be directed along. The well fluid flow path 850 may be in fluid communication with the plurality of wells 804. In some embodiments the well fluid flow path 850 may be in fluid communication with the first channel 802 or the second channel 802b or both.

Connected to the plurality of wells 804 may be a first fluid flow path 841 or a second fluid flow path 842 or both. In some embodiments, the first fluid flow path 841 is connected via a first side of the plurality of wells 804 to the plurality of wells 804 and the second fluid flow path 842 is connected via a second side of the plurality of wells 804 to the plurality of wells 804. The first fluid flow path 841 may be in fluid communication with a third channel 802c which may be fluidically coupled to an opening (not illustrated). The second fluid flow path 842 may be in fluid communication with a fourth channel 802d which may be fluidically coupled to an opening (not illustrated).

Contents within the well fluid flow path 850, the first fluid flow path 841, or the second fluid flow path 842, or any combination thereof may be directed along said flow paths via positive pressure (pressure above atmospheric pressure) pumping (such as through a positive pressure pump). Contents within the well fluid flow path 850, the first fluid flow path 841, or the second fluid flow path 842, or any combination thereof may be directed along said flow paths via negative pressure (pressure below atmospheric pressure) pumping (such as through a negative pressure pump). The well fluid flow path 850, the first fluid flow path 841, or the second fluid flow path 842, or any combination thereof may be subjected to a vacuum. The well fluid flow path 850, the first fluid flow path 841, or the second fluid flow path 842, or any combination thereof may be fluidically coupled to any pump described herein.

The support system 800 may alternatively be configured in several ways. In a first configuration, one or more droplets (which may be in a solution) may be directed along a well fluid flow path 850 while a first fluid phase is disposed within a first fluid flow path 841 and a second fluid phase is disposed within a second fluid flow path 842. The flow of the one or more droplets along the well fluid flow path 850 may be in any direction (e.g., into or out of the plurality of wells). Similarly, the flow of the first fluid phase along the first fluid flow path 841 or the flow of the second fluid phase along the second fluid flow path 842, or any combination thereof, may be in any direction. For example, the flow of the first fluid phase along the first fluid flow path 841 may be in a first direction while flow of the second fluid phase along the second fluid flow path 842 may be in a second direction, wherein the first and second directions are the same or the flow of the first fluid phase along the first fluid flow path 841 may be in a first direction while flow of the second fluid phase along the second fluid flow path 842 may be in a second direction, wherein the first and second directions are different (e.g., in opposite directions, in perpendicular directions, etc.). Moreover, the flow of the one or more droplets along the well fluid flow path 850, the flow of the first fluid phase along the first fluid flow path 841, or the flow of the second fluid phase along the second fluid flow path 842, or any combination thereof, may individually or collectively change directions, such as at the behest of a user or as directed by a controller. The flow of the one or more droplets along the well fluid flow path 850, the flow of the first fluid phase along the first fluid flow path 841, or the flow of the second fluid phase along the second fluid flow path 842, or any combination thereof, may individually or collectively change directions once during a procedure, two or more times during a procedure, and/or at constant intervals. The first fluid phase and the second fluid phase may be of any type of fluid described herein. In some embodiments, the first fluid phase may have a first fluidic property (e.g., density, viscosity (kinematic, dynamics, etc.), temperature, pressure, specific volume, specific weight, specific gravity, etc.) and the second fluid phase may have a second fluidic property (e.g., density, viscosity (kinematic, dynamics, etc.), temperature, pressure, specific volume, specific weight, specific gravity, etc.) that differs from the first fluidic property of the first fluid phase. For example, the first fluid phase may comprise a fluid with a first density greater than the density of the individual droplet from the plurality of droplets and the second fluid phase may comprise a fluid with a second density less than the density of the individual droplet from the plurality of droplets. As such, the individual droplet from the plurality of droplets may be retained within the individual well of the plurality of wells. One of skill in the art will appreciate that other such combinations of first fluidic properties and second fluidic properties may be used to retain the individual droplet within the individual well such as a first pressure and a second pressure, a first flow rate and a second flow rate, etc.

In another configuration, one or more droplets (which may be in a solution) may be directed along a well fluid flow path 850 while a first fluid phase is disposed within a first fluid flow path 841 and disposed within a second fluid flow path 842. The flow of the one or more droplets along the well fluid flow path 850 may be in any direction (e.g., into or out of the plurality of wells). Similarly, the flow of the first fluid phase along the first fluid flow path 841 or along the second fluid flow path 842, or any combination thereof, may be in any direction. For example, the flow of the first fluid phase along the first fluid flow path 841 may be in a first direction while flow of the first fluid phase along the second fluid flow path 842 may be in a second direction, wherein the first and second directions are the same or the flow of the first fluid phase along the first fluid flow path 841 may be in a first direction while flow of the first fluid phase along the second fluid flow path 842 may be in a second direction, wherein the first and second directions are different (e.g., in opposite directions, in perpendicular directions, etc.). Moreover, the flow of the one or more droplets along the well fluid flow path 850, the flow of the first fluid phase along the first fluid flow path 841, or the flow of the first fluid phase along the second fluid flow path 842, or any combination thereof, may individually or collectively change directions, such as at the behest of a user or as directed by a controller. The flow of the one or more droplets along the well fluid flow path 850, the flow of the first fluid phase along the first fluid flow path 841, or the flow of the first fluid phase along the second fluid flow path 842, or any combination thereof, may individually or collectively change directions once during a procedure, two or more times during a procedure, and/or at constant intervals.

In another configuration, one or more droplets (which may be in a solution) may be directed along a first fluid flow path 841 while a first fluid phase is disposed within a well fluid flow path 850 and a second fluid phase is disposed within a second fluid flow path 842. The flow of the one or more droplets along the first fluid flow path 841 may be in any direction (e.g., into or out of the plurality of wells). Similarly, the flow of the first fluid phase along the well fluid flow path 850 or the flow of the second fluid phase along the second fluid flow path 842, or any combination thereof, may be in any direction. For example, the flow of the first fluid phase along the first fluid flow path may be in a first direction while flow of the second fluid phase along the second fluid flow path may be in a second direction, wherein the first and second directions are the same or the flow of the first fluid phase along the first fluid flow path may be in a first direction while flow of the second fluid phase along the second fluid flow path may be in a second direction, wherein the first and second directions are different (e.g., in opposite directions, in perpendicular directions, etc.). Moreover, the flow of the one or more droplets along the first fluid flow path 841, the flow of the first fluid phase along the well fluid flow path 850, or the flow of the second fluid phase along the second fluid flow path 842, or any combination thereof, may individually or collectively change directions, such as at the behest of a user or as directed by a controller. The flow of the one or more droplets along the first fluid flow path 841, the flow of the first fluid phase along the well fluid flow path 850, or the flow of the second fluid phase along the second fluid flow path 842, or any combination thereof, may individually or collectively change directions once during a procedure, two or more times during a procedure, and/or at constant intervals.

In another configuration, one or more droplets (which may be in a solution) may be directed along a first fluid flow path 841 while a first fluid phase is disposed within a well fluid flow path 850 and disposed within a second fluid flow path 842. The flow of the one or more droplets along the first fluid flow path 841 may be in any direction (e.g., into or out of the plurality of wells). Similarly, the flow of the first fluid phase along the well fluid flow path 850 or along the second fluid flow path 842, or any combination thereof, may be in any direction. For example, the flow of the first fluid phase along the well fluid flow path 850 may be in a first direction while flow of the first fluid phase along the second fluid flow path 842 may be in a second direction, wherein the first and second directions are the same or the flow of the first fluid phase along the well fluid flow path 850 may be in a first direction while flow of the first fluid phase along the second fluid flow path 842 may be in a second direction, wherein the first and second directions are different (e.g., in opposite directions, in perpendicular directions, etc.). Moreover, the flow of the one or more droplets along the first fluid flow path 841, the flow of the first fluid phase along the well fluid flow path 850, or the flow of the first fluid phase along the second fluid flow path 842, or any combination thereof, may individually or collectively change directions, such as at the behest of a user or as directed by a controller. The flow of the one or more droplets along the first fluid flow path 841, the flow of the first fluid phase along the well fluid flow path 850, or the flow of the first fluid phase along the second fluid flow path 842, or any combination thereof, may individually or collectively change directions once during a procedure, two or more times during a procedure, and/or at constant intervals.

One or more droplets may directed through an opening 803a (for example, by first passing through a channel coupled to the coupling element 805a of FIG. 8A) through a channel 802a to the well fluid flow path 850 from which a given droplet selected from the one or more droplets may be directed into an individual well selected from the plurality of wells 804.

In some embodiments, one or more droplets (or a solution comprising one or more droplets) may be directed through a droplet flow opening 803b, along a droplet flow path 802b, until coming to an individual well from the plurality of wells 804 where it is held. Such embodiments may further comprise a first fluid phase directed through a first fluid flow opening 803a through a channel 802

FIG. 8C shows a close-up view of a subset of the plurality of wells 804 from the exemplary embodiment of the support system 800 comprising a plurality of wells 804 shown in FIG. 8A.

A first individual well 814 from the plurality of wells 804 may be in fluid communication with a first fluid flow path 841 via a first coupling channel 811b. At the interface between the first coupling channel 811b and the first fluid flow path 841 may be a first semipermeable membrane 861 as previously described (e.g., a semipermeable membrane comprising a hydrophobic surface). A second individual well 815 from the plurality of wells 804 may be in fluid communication with a second fluid flow path 842 via a second coupling channel 811d. At the interface between the second coupling channel 811d and the first fluid flow path 842 may be a second semipermeable membrane 862 as previously describe (e.g., a semipermeable membrane comprising a hydrophobic surface). The first individual well 814 may be in fluid communication with the second individual well 815 from the plurality of wells 804 via a first well-to-well channel. A first subset of the plurality of wells 804 may be fluidically coupled to a second set of the plurality of wells 804 through a well fluid flow path 850.

An individual well 814 from the plurality of wells 804 may take on many shapes, such as a shape that is generally circular, a shape that is generally oval-like, a shape that is generally tear-drop-like, a shape that is generally triangular, a shape that is generally square, a shape that is generally rectangular, a shape that comprises a polygon.

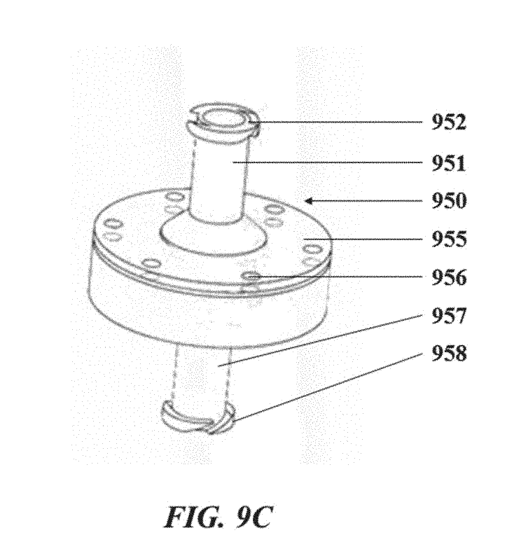

FIG. 9A shows a perspective view of an exemplary droplet generation system 900 comprising a reservoir 940 and a droplet generating apparatus 950. The reservoir 940 and the droplet generating apparatus 950 both couple to a base 910 of the system 900. Coupling of the reservoir 940 and/or the droplet generating apparatus 950 may comprise a physical interlocking of elements (such as a feature (e.g., a protrusion, a shaft, a set of threads, a channel, etc.) found on the reservoir 940 or the droplet generating apparatus 950 that has a corresponding matched feature (e.g., a divot, a hole, a receiving set of threads, a ring of material, etc.) found in the base 910), a pressed fit (also known as an interference fit) between the reservoir 940 and the base 910 and/or between the droplet generating apparatus 950 and the base 910, an adhesive bond (such as via chemical bonding, a glue, tape, etc.), a weld, or a solder. Coupling of the reservoir 940 and/or the droplet generating apparatus 950 or both to the base 910 may be aided by a coupling mechanism 920 that may help clamp the reservoir 940 and/or the droplet generating apparatus 950 or both to the base 910. Coupling of the reservoir 940 and/or the droplet generating apparatus 950 to the base 910 may comprise releasable coupling wherein the reservoir 940 and/or the droplet generating apparatus 950 may be removed from the base 910. As such, coupling between the reservoir 940 and/or the droplet generating apparatus 950 to the base 910 may be temporary, permanent, or operative, or any combination thereof.

The reservoir 940 may comprise a shaft 941 that terminates at a distal end 942. The shaft 941 may be an elongate shaft. The shaft 941 may take on any cross-sectional shape or size. In some embodiments, the shaft 941 may be shaped and/or sized to approximate the shape and/or size of a syringe. The distal end 942 of the reservoir 940 may comprise a coupling mechanism. The coupling mechanism may be of any type described herein (e.g., a set of threads). In some embodiments the distal end 942 of the reservoir 940 couples to a source of fluid such as a tube or a syringe. Coupling to the distal end 942 of the reservoir 940 may comprise threadable engagement between the distal end 942 and that which is coupling to it such that at least a first direction of threadable engagement (e.g., a first rotation, such as clockwise) causes further engagement (e.g., a tightening, an increase in amount of threads engaged, an increase in the amount of distal end 942 covered, an increase in the force and/or work and/or energy required to decouple, etc.) between the distal end 942 and that which is coupling to it and at least a second direction of threadable engagement (e.g., a second rotation, such as counterclockwise) causes less engagement (e.g., a loosening, a decrease in the amount of threads engaged, a decrease in the amount of distal end 942 covered, a decrease in the force and/or work and/or energy required to decouple, etc.) between the distal end 942 and that which is coupling to it. In some embodiments, the distal end 942 is left free and open to the surrounding environment.

In some embodiments, the reservoir 940 comprises a syringe. The syringe may be of any type known in the art including passively actuated and actively actuated syringes. In some embodiments, the reservoir's 940 contents (e.g., a first fluid phase, a second fluid phase, etc.) may be mobilized by the active control of a controller. In some embodiments, the reservoir's 940 contents (e.g., a first fluid phase, a second fluid phase, etc.) may be mobilized by a user (such as by depressing a plunger (as is the case with syringe embodiments) or by adding additional contents).

The droplet generating apparatus 950 may comprise a shaft 951 that terminates at a distal end 952. The shaft 951 may be an elongate shaft. The shaft 951 may take on any cross-sectional shape or size. In some embodiments, the shaft 951 may be shaped and/or sized to approximate the shape and/or size of a syringe. The distal end 952 of the droplet generating apparatus 950 may comprise a coupling mechanism. The coupling mechanism may be of any type described herein (e.g., a set of threads). In some embodiments the distal end 952 of the droplet generating apparatus 950 couples to a source of fluid such as a tube or a syringe. Coupling to the distal end 952 of the droplet generating apparatus 950 may comprise threadable engagement between the distal end 952 and that which is coupling to it such that at least a first direction of threadable engagement (e.g., a first rotation, such as clockwise) causes further engagement (e.g., a tightening, an increase in amount of threads engaged, an increase in the amount of distal end 952 covered, an increase in the force and/or work and/or energy required to decouple, etc.) between the distal end 952 and that which is coupling to it and at least a second direction of threadable engagement (e.g., a second rotation, such as counterclockwise) causes less engagement (e.g., a loosening, a decrease in the amount of threads engaged, a decrease in the amount of distal end 952 covered, a decrease in the force and/or work and/or energy required to decouple, etc.) between the distal end 952 and that which is coupling to it. In some embodiments, the distal end 952 is left free and open to the surrounding environment.

The droplet generating apparatus 955 may further comprise a container 955. The container 955 may have internal features (such as those shown in FIG. 9B) such as a first chamber or a second chamber. The container 955 may comprise a membrane. The container 955 of some embodiments at some points in time comprises a first fluid phase (e.g., an oil). In some embodiments, the container 955 comprises a second fluid phase (e.g., an aqueous solution) at some points in time. At some points in time in some embodiments, the container 955 comprises reagents necessary for a chemical or biological reaction.

FIG. 9B shows a cut side view of the exemplary droplet generation system 900 shown in FIG. 9A, emphasizing internal features. The exemplary droplet generation system 900 comprises a reservoir 940 and a droplet generating apparatus 950 (such as that seen in FIG. 1 and that seen in FIGS. 7A-7D).

The reservoir 940 may further comprise a channel 944. The channel 944 may be sized and/or shaped to receive a syringe.

Any number of fluid phases (e.g., a first fluid phase, a second fluid phase, a third fluid phase, etc.) may be individually or collectively, simultaneously or sequentially, directed to move from the distal end 942 of the reservoir 940, through the channel 944 of the reservoir 942, through the proximal end 943 of the channel 944, through the opening 949 of the reservoir 940 that connects to the channel 930 of the base 910, along the channel 930 to the opening 959 of the droplet generating apparatus 950, through the first chamber 961 of the droplet generating apparatus 950, through the membrane 965 into the second chamber 962 of droplet generating apparatus 950, and through the opening 953 to the channel 954 of the droplet generating apparatus 950. Once in the channel 954, the fluid phase(s) (which may comprise one or more droplets if, while flowing along the aforementioned fluid flow path, the fluid phase(s) undergo a method of droplet generation as described herein) may be further directed out of the system (e.g., for further processing, sequencing, detection, etc.).

In some embodiments, any number of fluid phases (e.g. a first fluid phase, a second fluid phase, a third fluid phase, etc.) may be individually or collectively, simultaneously or sequentially, directed to move from the distal end 952 of the droplet generating apparatus 950, through the channel 954, through the opening 953 of the container 955, into the second chamber 962 of the droplet generating apparatus 950, through the membrane 965, into the first chamber 961, through the opening 959 of the droplet generating apparatus that connects to channel 930 of the base 910, through the channel 930 to the opening 949 of the reservoir 940, through the proximal end 943 of the reservoir 940, through the channel 944, to the distal end 942 of the reservoir. Once in the channel 944, the fluid(s) (which may comprise one or more droplets if, while flowing along the aforementioned fluid flow path, the fluid phase(s) undergo a method of droplet generation as described herein) may be further directed out of the system (e.g., for further processing, sequencing, detection, etc.).

Droplets may be generated with the droplet generation system 900 in accordance with the descriptions of any of the methods or systems of droplet generation described herein.

FIG. 9C shows a perspective view of the droplet generating apparatus 950 of the droplet generation system 900 shown in FIG. 9A. As previously described, the droplet generating apparatus 950 may be removable from the droplet generation system 900. The droplet generating apparatus 950 comprises a first shaft 951 that terminates in a distal end 952, second shaft 957 that terminates at a proximal end 968, and a container 955 to which the first shaft 951 and the second shaft 957 couple. The interior of the first shaft 951 and/or interior of the second shaft 957 may be in fluidic communication with the interior of the container 955. (For an internal view of the container 955 see FIG. 9B.)

The distal end 952 of the first shaft 951 may be of any type described herein, such as one that terminates in a set of threads for threadably engaging another element (e.g., a syringe, a tube, a vessel, a pump, etc.). The proximal end 958 of the second shaft 957 may terminate in a set of threads for threadably engaging another element (e.g., a syringe, a tube, a vessel, a pump, etc.).

The container 955 may comprise two or more components that when brought together come to define the interior of the container (e.g., an interior comprise one or more chambers, an interior that comprises a membrane, etc.). For those embodiments of the container 955 comprising two or more components, the two or more components may be coupled together via any method of coupling described herein, such as fastening one or more fasteners 956 (e.g., one or more screws, one or more bolts, one or more pins, etc.).

The system 900 may further comprise a detector (not illustrated) in sensing communication with the system 900 such that a detectable moiety (of any type described herein) may be detected.

The system 900 may further comprise a vibrator (not illustrated) in physical contact with the system 900 that may vibrate at least a portion of the system 900 to aid in droplet formation, detachment from the membrane, and/or guidance along one or more channels.

### Control Systems

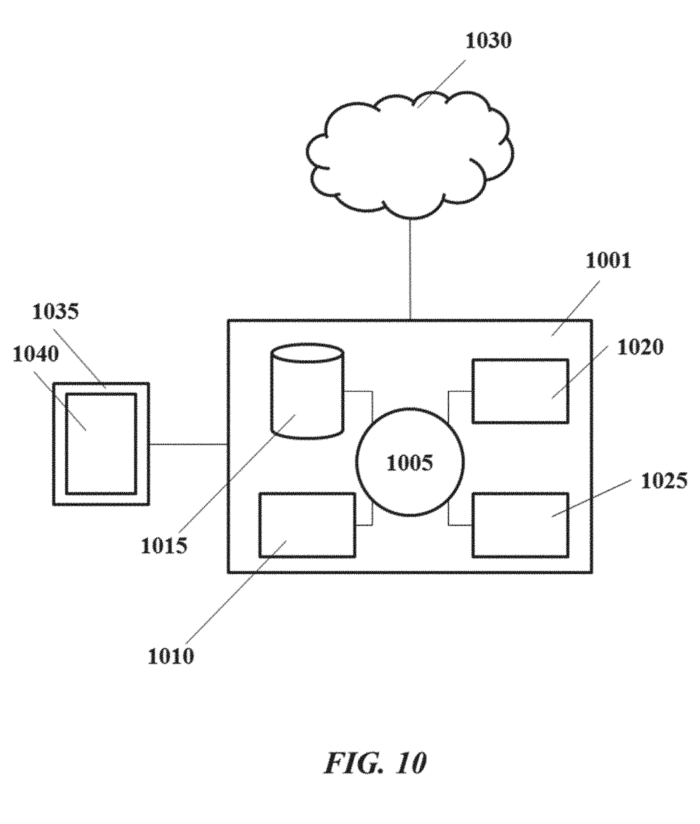

The present disclosure provides computer control systems that are programmed to implement methods of the disclosure. FIG. 10 shows a computer system 1001 that is programmed or otherwise configured for sample processing and analysis, such as droplet generation and nucleic acid amplification and detection. The computer system 1001 can regulate various aspects of methods and systems of the present disclosure.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

In another aspect, the present disclosure provides a non-transitory computer-readable medium comprising machine executable code that, upon execution by one or more computer processors, implements a method for conducting a chemical or biological reaction on a biological sample. The method may comprise depositing a solution comprising the biological sample in a sample holder, and the sample holder may retain the solution during the chemical or biological reaction. The sample holder may be disposed adjacent to a plurality of thermal zones comprising at least a first thermal zone and a second thermal zone. The second thermal zone may be angularly separated from the first thermal zone along an axis of rotation of (1) the sample holder or (2) the plurality of thermal zones. The method may further comprise alternately and sequentially positioning the solution in each of the plurality of thermal zones through rotation of the sample holder or the plurality of thermal zones, to conduct the chemical or biological reaction on the biological sample. In the first thermal zone, the solution may be subjected to heating or cooling at a first temperature profile, and in the second thermal zone, the solution may be subjected to heating or cooling at a second temperature profile that is different than the first temperature profile.

In another aspect, the present disclosure provides a non-transitory computer-readable medium comprising machine executable code that, upon execution by one or more computer processors, implements a method for generating at least one droplet comprising a biological sample for use in a chemical or biological reaction. The method may comprise activating an apparatus comprising (1) a first chamber comprising a first fluid volume and at least one first fluid flow port that is in fluid communication with the first fluid volume, wherein the first fluid volume comprises an aqueous solution comprising the biological sample for use in the chemical or biological reaction; and (2) a second chamber comprising a second fluid volume and at least one second fluid flow port that is in fluid communication with the second fluid volume, wherein the second chamber at least partially circumscribes the first chamber, wherein the second fluid volume retains a continuous fluid that is immiscible with the aqueous solution, and wherein the second chamber is rotatable with respect to the first chamber, or vice versa. The method may further comprise rotating the first chamber or the second chamber to bring the first fluid flow port in alignment with the second fluid flow port to subject the aqueous solution comprising the biological sample to flow from the first fluid volume to the second fluid volume to generate the at least one droplet upon the aqueous solution contacting the continuous fluid, which at least one droplet comprises the biological sample or a portion thereof.

In another aspect, the present disclosure provides a non-transitory computer-readable medium comprising machine executable code that, upon execution by one or more computer processors, implements a method for cooling a solution comprising a biological sample (e.g., nucleic acid sample) during a chemical or biological reaction (e.g., nucleic acid amplification reaction).

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, nucleic acid sequence information. Examples of UI's include, without limitation, a graphical user interface (GUI) and web-based user interface. In some cases, a user interface can be a graphical user interface. Moreover, a user interface can include one or more graphical elements. Graphical elements can include image and/or textual information, such as pictures, icons and text. The graphical elements can have various sizes and orientations on the user interface. Furthermore, an electronic display screen may be any suitable electronic display including examples described elsewhere herein. Non-limiting examples of electronic display screens include a monitor, a mobile device screen, a laptop computer screen, a television, a portable video game system screen and a calculator screen. In some embodiments, an electronic display screen may include a touch screen (e.g., a capacitive or resistive touch screen) such that graphical elements displayed on a user interface of the electronic display screen can be selected via user touch with the electronic display screen.

In some embodiments, a user interface can be used to select a protocol for the system. For example, the user interface that may display one or more graphical elements accessible by a user to execute an amplification protocol to amplify the target nucleic acid in the biological sample. As another non-limiting example, the user interface may display one or more graphical elements accessible by a user to execute a temperature monitoring function. Such temperature monitoring functions may be of any sort described herein such as displaying a current temperature value, displaying a desired temperature value, displaying a current heat flux, display a desired heat flux, allowing a user to select a desired temperature (after which the controller may direct that heating and/or cooling of one or more heating elements). The user interface may be used to allow a user to direct any actions of any stems described herein including but not limited to amplifying a chemical or biological product, directing a chemical or biological reaction (e.g., to occur, to occur at a desired rate, etc.), detecting a chemical or biological reaction and/or the products thereof, etc.

The system may also comprise a computer processor 1005 coupled to the electronic display screen and programmed to execute an amplification protocol upon selection of the graphical element by the user. The amplification protocol can comprise subjecting a reaction mixture comprising the biological sample and reagents necessary for conducting nucleic acid amplification to a plurality of series of primer extension reactions to generate amplified product that is indicative of the presence of the target nucleic acid in the biological sample. Each series of primer extension reactions can include two or more cycles of incubating the reaction mixture under a denaturing condition characterized by a denaturing temperature and a denaturing duration, followed by incubating the reaction mixture under an elongation condition characterized by an elongation temperature and an elongation duration. An individual series may differ from at least one other individual series of the plurality with respect to the denaturing condition and/or the elongation condition.

In some embodiments, the amplification protocol can further comprise selecting a primer set for the target nucleic acid. In some embodiments, the reagents may comprise a deoxyribonucleic acid (DNA) polymerase, an optional reverse transcriptase, and a primer set for the target nucleic acid. In some embodiments, the user interface can display a plurality of graphical elements. Each of the graphical elements can be associated with a given amplification protocol among a plurality of amplification protocols. In some embodiments, each of the graphical elements may be associated with a disease. A given amplification protocol among the plurality of amplification protocols can be directed to assaying a presence of the disease in the subject.

Methods and systems of the present disclosure can be implemented by way of one or more algorithms. An algorithm can be implemented by way of software upon execution by the central processing unit 1005. The algorithm can, for example, regulate systems or implement methods provided herein.

Devices, systems and methods of the present disclosure may be combined with other devices, systems or methods, such as those described in PCT/CN14/094914 and PCT/CN14/078022, each of which is entirely incorporated herein by reference.

### EXAMPLES

### Example 1: Droplet Generation, Droplet Size as a Function of Flow Rate

The droplet size of the plurality of droplets may be at least partially a function of the flow rate of the first fluid phase, the second fluid phase, or both. Though droplets may be generated in accordance with any method described herein, specifically in this particular example one of two protocols may be used. A first method using a syringe pump and a droplet generating apparatus as described herein comprises: (a) filling the chamber with a fluorinated oil of certain volume (for example, about 200 uL); (b) filling the syringe with a volume of fluorinated oil and an aqueous solution (since there may be “dead volume” in the container just below the membrane, the use of oil here ensures that all of the aqueous solution is pushed through the membrane); (c) coupling the syringe with the aqueous solution to the droplet generation system (of any type described herein); (d) coupling the system to a syringe pump such that the syringe is caused to mobilize its contents when acting upon by the syringe pump and set a flow rate; and (e) pushing air (if present), water and oil in the syringe through the membrane, such that droplets are formed. The droplets may flow up to an oil-air surface. A second method using a pressure pump and a droplet generating apparatus as described herein comprises: (a) filling the chamber with fluorinated oil and an aqueous solution (for example, about 200 uL); (b) connecting the droplets collector to the left part of device; (c) connecting the pressure controller to the right part above the fluids; (d) applying a constant pressure; and (e) pushing air (if present), oil and water through the entrance region through to the membrane. The fluorinated oil should stay in good contact with the membrane. Droplets are formed by passing the aqueous solution though membrane until it contacts the oil phase. The droplets may flow up to the oil-air surface.

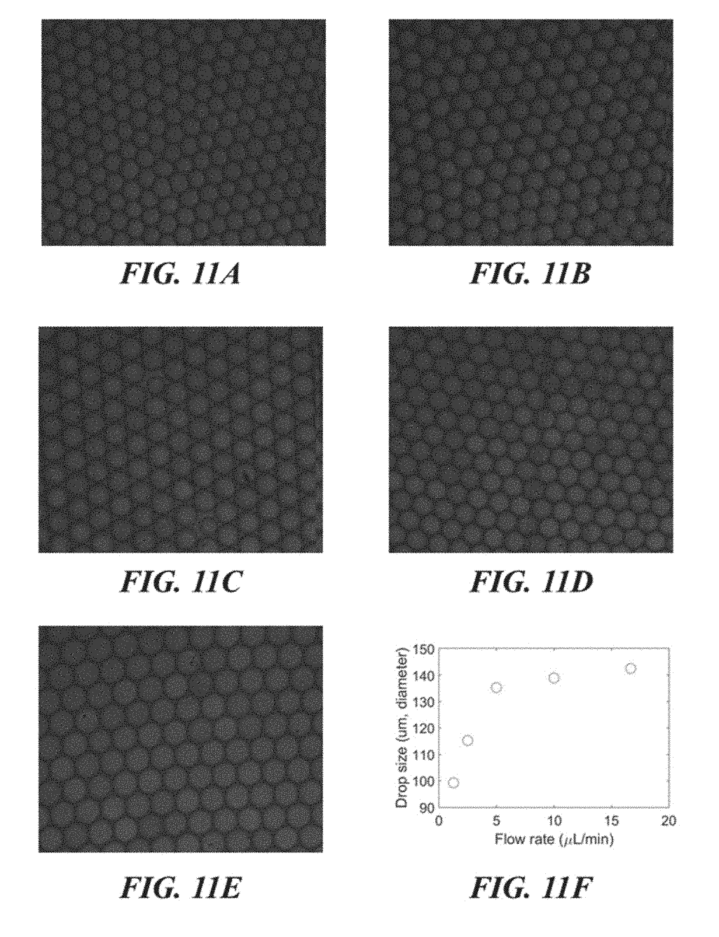

Five flow rates were tested: 75 microliters per hour (μl/hr), 150 μl/hr, 300 μl/hr, 600 μl/hr, and 1000 μl/hr. Images of the plurality of droplets formed for each of these rates may be found in FIGS. 11A-11E. More specifically, FIG. 11A shows the plurality of droplets formed from the experiment wherein the flow rate was 75 μl/hr; FIG. 11B shows the plurality of droplets formed from the experiment wherein the flow rate was 150 μl/hr; FIG. 11C shows the plurality of droplets formed from the experiment wherein the flow rate was 300 μl/hr; FIG. 11D shows the plurality of droplets formed from the experiment wherein the flow rate was 600 μl/hr; and FIG. 11E shows the plurality of droplets formed from the experiment wherein the flow rate was 1000 μl/hr.

A relationship was found wherein the droplet size increased as a function of the flow rate such that a flow rate of 75 μl/hr produced droplets with an average diameter of approximately 99.2 micrometers (μm), a flow rate of 150 μl/hr produced droplets with an average diameter of approximately 115.2 μm, a flow rate of 300 μl/hr produced droplets with an average diameter of approximately 135.2 μm, a flow rate of 600 μl/hr produced droplets with an average diameter of approximately 138.8 μm, a flow rate of 1000 μl/hr produced droplets with an average diameter of approximately 142.2 μm. A graphical representation of this relationship may be seen in FIG. 11F. From FIG. 11F one can see that the relationship between flow rate and droplet size may be non-linear.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for facilitating a chemical or biological reaction on a biological sample, comprising:
subjecting a first fluid phase to flow along a fluid flow path, through at least one opening in a membrane, to a chamber downstream of said membrane, wherein said membrane intersects said fluid flow path, and wherein said membrane is flexible;
subjecting a second fluid phase to flow along said fluid flow path through said at least one opening in said membrane to said chamber, which chamber comprises said first fluid phase that is immiscible with said second fluid phase, wherein said second fluid phase comprises said biological sample or a portion of said biological sample; and
generating a plurality of droplets in said chamber upon said second fluid phase coming in contact with said first fluid phase, wherein a given droplet of said plurality of droplets comprises said biological sample and reagents necessary for said chemical or biological reaction.

2. The method of claim 1, wherein said first fluid phase and/or said second fluid phase is directed using a flow controller, a positive pressure or a negative pressure.

3.-4. (canceled)

5. The method of claim 1, wherein said first or second fluid phase comprises reagents necessary for the chemical or biological reaction.

6. (canceled)

7. The method of claim 1, wherein said chemical or biological reaction is nucleic acid amplification, and wherein said reagents include one or more primers and polymerizing enzyme.

8. The method of claim 7, wherein said nucleic acid amplification is polymerase chain reaction (PCR).

9. (canceled)

10. The method of claim 7, further comprising subjecting said given droplet to nucleic acid amplification under conditions necessary to generate amplification product(s) from said biological sample in said given droplet.

11.-12. (canceled)

13. The method of claim 7, wherein said biological sample comprises a virus.

14.-15. (canceled)

16. The method of claim 13, wherein said virus is selected from the group consisting of human immunodeficiency virus I (HIV I), human immunodeficiency virus II (HIV II), an orthomyxovirus, Ebola virus, Dengue virus, influenza viruses, herpesvirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, Coxsackie virus, papillomavirus, zika virus, and Varicella virus.

17.-20. (canceled)

21. The method of claim 7, wherein said biological sample comprises a pathogenic bacterium or a pathogenic protozoan.

22. (canceled)

23. The method of claim 21, wherein said pathogenic bacterium is selected from the group consisting of Staphylococcus aureus, Listeria monocytogenes, Escherichia coli, Enterobacter sakazakii, Vibrio Parahemolyticus, and Shigella spp, Mycobacterium tuberculosis, Plasmodium, and Salmonella

24.-26. (canceled)

27. The method of claim 1, further comprising detecting said amplification product(s) in said given droplet.

28. The method of claim 1, further comprising monitoring a temperature of a solution comprising said plurality of droplets.

29. (canceled)

30. The method of claim 1, wherein each of said plurality of droplets has a droplet size from about 0.1 micrometers to about 200 micrometers.

31.-35. (canceled)

36. The method of claim 1, wherein said first fluid phase comprises an oil.

37. The method of claim 36, wherein said first fluid phase comprises a surfactant.

38.-39. (canceled)

40. The method of claim 1, further comprising subjecting said chamber to vibration.

41.-43. (canceled)

44. The method of claim 1, wherein said membrane includes a lipid bilayer.

45. The method of claim 1, wherein said at least one opening includes a pore protein.

46. The method of claim 45, wherein said pore protein is alpha hemolysin or a variant thereof.

47. A system for conducting a chemical or biological reaction on a biological sample, comprising:
a fluid flow path in fluid communication with a chamber downstream of a membrane, wherein said membrane comprises at least one opening and intersects said fluid flow path, and wherein said membrane is flexible;
a controller comprising one or more computer processors that are individually or collectively programmed to:
(i) subject a first fluid phase to flow along said fluid flow path, through said at least one opening in said membrane, to said chamber downstream of said membrane;
(ii) subject a second fluid phase to flow along said fluid flow path through said at least one opening in said membrane to said chamber, which chamber comprises said first fluid phase that is immiscible with said second fluid phase, wherein said second fluid phase comprises said biological sample or a portion of said biological sample; and
(iii) generate a plurality of droplets in said chamber upon said second fluid phase coming in contact with said first fluid phase,
wherein a given droplet of said plurality of droplets comprises said biological sample and reagents necessary for said chemical or biological reaction.

48.-88. (canceled)
